SEQUENCE LISTING (149 sequences)

SEQ ID NO: 1 - protein, 62 aa, Acinetobacter baumannii
MDPETHCHAERCFLSWFCGNQLPAYKCFQITWFVSWTPCPDCVAKLAEFLSEHPNVTLTISA

SEQ ID NO: 2 - protein, 192 aa, Alligator mississippiensis
MADSSEKMRGQYISRDTFEKNYKPIDGTKEAHLLCEIKWGKYGKPWLHWCQNQRMNIHAEDYFMNNIFKAKKHPVHCYVTWYLSWSPCADCASKIVKFLEERPYLKLTIYVAQLYYHTEEENRKGLRLLRSKKVIIRVMDISDYNYCWKVFVSNQNGNEDYWPLQFDPWVKENYSRLLDIFWESKCRSPNPW

SEQ ID NO: 3 - protein, 228 aa, Alligator sinensis
MAEKQENPENTQNGEPETENAELTEEEKEKEKERKKKELEELPPFEIVSGERLPAFFFKFQFKNVEYSSGRNKTFLCYIVETQGKESGTIRGYLEDEHAAAHAEDAFFNTILPECESSLRYNVTWYVSSSPCVACAERIAEILKKNKNLRLTILVSRLFMWEEPEMQAVLKKMKAAGCKLRIMKPQDFEYVWQNFVEQEEGESKDFVPWEDIQENFLYYEEKLAEVLH

SEQ ID NO: 4 - protein, 198 aa, Alligator sinensis
MDSLLMKQKKFLYNFKNLRWAKGRHETYMCYVVKRRDSATSCSLDFGYLRNKSGCHVEVLFLRYISAWDLDPGRCYRVTWFTSWSPCYDCARHVADFLRAYPNLTLRIFVARLYFCEGRNAEPEGLRRLHRAGAHIAIMTFKDYFYCWNTFVENRERTFKAWEGLHENSVHLTRKLRRILLPLYEVDDLRDAFRTLGL

SEQ ID NO: 5 - protein, 174 aa, Anolis carolinensis
MQGWKLEAEEFLSNFGPSEHPEVTYLLYEIQWGRSRRTWKNWCRNIHPEHAEIIFLEDEEVHDRPDIPCNITWFVPWTPCGACAYEIIAFLEERPNVNLEILAAQVYKPYDHRNRKGLRDLADEAQISIMEFSDYQYCWEKFVDHQGMNLRAGRGFSNQRRIDSRQLDSIINPF

SEQ ID NO: 6 - protein, 194 aa, Balaenoptera acutorostrata
MEASTAPGTRCLLDENTFTENFMNQISVRKTYLCYKVEILDGDAWVPLDEYKGFVHNKGANQPGQPCHAELYFLDRIRSWNLDQELHYRLTCFISWTPCHTCAQNLATFLGENSHVSLRIFASRIYSLAGYEAGLRTLQAAGAQIAIMTSKEFEHCWETFVDHQGRPFQPWDELEVVSQHLCEKLQAILQTQQN

SEQ ID NO: 7 - protein, 246 aa, Boleophthalmus pectinirostris
MADKSRLAMRRKDKKEELEPVKNVKEVKEVKEEEGEKPAENGEAPQENGAAAENGEPLLEPMELPPFEIITGDRIDPFVFKFQFKNVEYSSGRNKTFLCYLVDRGSVEGLLRGYLEDEHTGAHAEEAFFTQCLPDYDPAVKYTITWYVSSSPCAACAAKIAEVLKTRKTVKLSIFASRLFEWEEPEIQAGLKTLHAAGCKLRMMKPLDFSYTWDTFVEGDEEAFNLWEDCKDNYEYYQDKLADILQ

SEQ ID NO: 8 - protein, 220 aa, Chelonia mydas
MAENQEKPSDTHNGEEQPENAEKPEEKKKELEKLPPFEIVRGERFPAFFFKFQFRNVEYSSGRNKTFLCYVVEIQGKEPGSFQGYLEDEHAAAHAEDAFFNNILPTCDPNLRYNVTWYVSSSPCVPCADRIAQVLQKNKNLRLSILVSRLFMWEEPEMQAALKKLKASGCKLKIMKPQDFEYIWQNFAEQEEGEAKAFEPWEDIQENFLYYEEKLSEVLH

SEQ ID NO: 9 - protein, 189 aa, Chrysemys picta
MFYEIKWSNSKRSWQSCCHSTRTEHAEIHFIEDVFQEQRSDPSVHYSITWYMSWSPCADCCKEIRDFLKEQPNVNLVIYVARLYWHKEETNRQGLRSLVNIGVSIRVMDLPAYSYCWRTFVNDEDKDEDDYWPRHFAPWIMLYSLELQSILQNIPSCLEISTDENQTPVFSLCVEDDQQKRALTSANPR

SEQ ID NO: 10 - protein, 198 aa, Crocodylus porosus
MDSLLMKQKKFLYNFRNLRRAKGRHETYLCYVVKRRDSATSCSLDFGYLRNKSGCHVEMIFLRYISAWDLDPGRCYRVTWFTSWSPCYDCARHVVDFLRAYPNLTLRIFVARLYFCEGRNAEPEGLRRLHRAGAHIAIMTFKDYFYCWNTFVENRERTFKAWEGLHENSVHLTRKLRRILLPLYEVDDLRDAFRTLGL

SEQ ID NO: 11 - protein, 228 aa, Crocodylus porosus
MAEKQENPENTQNGEPEPENAELTEEEKEKEKERKKKELEELPPFEIVSGERLPAFFFKFQFKNVEYSSGRNKTFLCYIVETQGKESGTIRGYLEDEHAAAHAEDAFFNTILPECESSLRYNVTWYVSSSPCVPCAERIAEILKKNKNLRLTILVSRLFMWEEPEMQAVLKKVKAAGCKLRIMKPQDFEYVWQNFVEQEEGESKDFVPWEDIQENFLYYEEKLAEVLH

SEQ ID NO: 12 - protein, 251 aa, Cynoglossus semilaevis
MADKGRAVARRKDRKEPDAADRAKSGKEEENIPEKTENNEGAPAAEEPAGGAVANGQNGEFKVEPMELPPFEIIQGDRIDAFAFKFQFKNVEYSSGRNKTFLCYLVDKGKASDGLLRGYLEDEHSGAHAEEAFFIQCLPDYDPALKYTITWYVSSSPCSACAEKIAEVLKARKNIKLSIFAARLFEWEEAEIQAGMKSLNAAGCKLRVMKPLDFSYIWDTFVENEDQPLNLWEDCKDNYEYYHEKLSEILQ

SEQ ID NO: 13 - protein, 210 aa, Cyprinus carpio
MISKLDSVLMTQKKFIFNYKNVRWARGRHDTYLCFVVKRRTGPDSLSFDFGHLRNRTGCHVELLFLRHLGALCPGLSGSSVDGARMYYAVTWFCSWSPCSKCSQQLAHFLSQTPNLRLRIFVSRLYFCDEEDSQEREGLRHLKRAGVQISVMTYKDYFYCWQTFVARRERSFKAWDDLHQNSVRLVRKLNRILQPCETEDLRDGFALLGL

SEQ ID NO: 14 - protein, 210 aa, Danio rerio
MICKLDSVLMTQKKFIFHYKNVRWARGRHETYLCFVVKRRIGPDSLSFDFGHLRNRSGCHVELLFLRHLGALCPGLSASSVDGARLCYSVTWFCSWSPCSKCAQQLAHFLSQTPNLRLRIFVSRLYFCDEEDSVEREGLRHLKRAGVQISVMTYKDFFYCWQTFVARRERSFKAWDGLHENSVRLVRKLNRILQPCETEDLRDVFALLGL

SEQ ID NO: 15 - protein, 249 aa, Danio rerio
MADRKGSSGVSSRLSVRRKEKAENDAKKEEKSPTEAEKPEVNGKEVPMENGEAGAAAADGEKPEPIELPPFETITGDRMDPFFFKFQFKNVEYSSGRNKTFLCYLVDHGGEGLMRGYIEDEHAGGHAEEAFFQQILTNYDPACKYTITWYMSSSPCANCATKLAEILRSRKNIRLAIFSSRLFEWEEPEIQAGLKLLASVGCKLRMMKPLDFTYTWDTFVESDEQQFTPWEDCQENYEYYQDKLADILQ

SEQ ID NO: 16 - protein, 190 aa, Dasypus novemcinctus
MEGSAEPKPSDLLSPEDFTFHYENLKYAHERRKTYLCYHLERLDGDRRVMLDSAVIQNQGSRHVEECFLFSENVLEKLKAPGSYSVTWYLSWSPCARCAQAVIRFLQQHPNVRLRILVARLYFHETRANRAALRALRRREAPIEIMSPEDFAYCWKHFVDHRGEDFQPWEDMDANVDRLAEELSGIWLSK

SEQ ID NO: 17 - protein, 210 aa, Denticeps clupeoides
MITKLDSVLMTQKKFIYHYKNLRWARGRCETYLCFVVKRRIGPNSLSFDFGHLRNRSGCHVELLFLRHLGALCPGLWGSGPGEVKAFYSVTWFCSWSPCSDCSRRVANFLLRTPNLRLRIFVARLYFCDLEDSLEREGLRTLKRAGVHLSVMAYKDYYYCWQTFVARRQRAFKAWDGLLQNSVRLARKLNRILQPCETEDLRDAFALLGL

SEQ ID NO: 18 - protein, 199 aa, Gavialis gangeticus
MAVGEKKGLLGTSQRWKIELKSFQENYMPSTWPKVTHLLYEIRWGKGSKVWRNRCSNTLTQHAEVNCLENAFGKLQFNHPVPCHITWFLSWSPCGRCCRRILQFLQAHPHVTLVIKAAQLFKHMDERNRQGLRDLVQNGVHVQVMDLPDYRYCWRTFVTHPHEGESDFWPWFFPLWITFYNQELQHILLVSRYFGKNVG

SEQ ID NO: 19 - protein, 208 aa, Lagenorhynchus obliquidens
MLPHFLPPLTHSLLMKRRKFLYHFKNVRWAKGRHETYLCYVVKRRDSATSFSLDFGHLRNKAGCHVELLFLRYISDWDLDPGRCYRVTWFTSWSPCYDCARHVADFLRGYPNLSLRIFTARLYFCDKERKAEPEGLRRLHRAGVQIAIMTFKDYFYCWNTFVENHERTFKAWEGLHENSVRLSRQLRRILLPLYEVDDLRDAFRTLGL

SEQ ID NO: 20 - protein, 146 aa, Latimeria chalumnae
MDSLLMQKKKFLYHYKNVRWARGRHETYLCYIVKRRYNPASYSLDFGFLRNKSGCHVEMLFLRFLTGWNIDPTLPYSVTWFTSWSPCYDCSQHVTHFLRVYPNLRLRIFTARLYFCEENNAEPEGLRNLHMAGVQLGVMTFKGKGG

SEQ ID NO: 21 - protein, 235 aa, Lipotes vexillifer
MASDRGPSAGDATSRRRIEPWEFEVSFDPRELCKETRLLYEIKWGKSQHVWRHSSKNTTKHVERNVIEKITSERRFQPSVSCCIIWFLSWSPCWECSKAIREFLNQHPRVTLIIYVARLFQHMDPQNRQGLRDLIHSGVTIQIMGPPEYDYCWRNFVNYPPGEEAHWPRYPPLLLKLYVLELHCIILGLPPCLNISRCQNQLTLFRLIPQNCHYQMIPPHILLHTGLIQLPMTWR

SEQ ID NO: 22 - protein, 194 aa, Nanorana parkeri
MDSLLLKRNKFLYHYKNMRWARGRHETYLCYIVKRRYSSVSTSMDFGYLRNRSGCHAEILFLRYLAIWLGHDPNRMFRVTWFSSWSPCRDCSQRTADFLVNHPNLSLRIFSARLYFCEEHTAEPEGLRKLQRAGVQVAYYFYCWNNFVENRERKFEAWDGLHENFVRLSRKLRRILQPPYDMEDLHEAFDLLGL

SEQ ID NO: 23 - protein, 218 aa, Neophocaena asiaeorientalis
MWGAEGKGVRGGEERWAVPEGAPRLRASPAFDRSCLLDENTFTENFMNRLRPRKTYLCYKVEILDGDARVPLDEKKGFVRNKGANEPGMPRHAECYFLDRIRSWNLDRGLHYRLTCFISWTPCHSCAQELATFLGENSHVSLHIFASRIYRRPGYEAGILTLRAAGAQIAIMTSKEFQHCWENFVDHQERPFRPWVGLEVESQHQCNELQAILQTQAN

SEQ ID NO: 24 - protein, 141 aa, Orcinus orca
MKRRKFLYHFKNVRWAKGRHETYLCYVVKRRDSATSFSLDFGHLRNKAGCHVELLFLRYISDWDLDPGRCYRVTWFTSWSPCYDCARHVADFLRGYPNLSLRIFTARLYFCDKERKAEPEGLRRLHRAGVQIAIMTFKAPI

SEQ ID NO: 25 - protein, 276 aa, Paramormyrops kingsleyae
MSDKKEGKAPGKLPGRKKMEKKADKAPEKEKVEKKVERKMEKDPDKAVEKVPTEKATENAPEVAEGTVGNGAPSEGAVASGGGEEFVVEPMELPPFEIIVGEKINPFFFKFQFKNVEYSSGRNKTFLCFLVDAQNSNGEGLRGYLEDEHTGAHAEEAFFHQVLPQYDPALRYMVTWYVSSSPCAACAAKLTEILKTRKTLHLTIFSSRLFMWEEPEIQVGLKALVAAGCKLRMMKPSDFIYTWDTFVENEDQSFTPWEDCQENYEYYQERLADILQ

SEQ ID NO: 26 - protein, 218 aa, Pelodiscus sinensis
MELLCTVNSLAAVQLQSKRVCRSLLMKQKKFLYNFKNLRWARGRHETYLCYVVKRRDSATSFSLDFGYLRNKSGCHVEMLFLRYISAWDLDPGRCYRVTWFTSWSPCYDCARHVADFLRAYPNLTLRIFAARLYFCEDRNAEPEGLRRLHRAGVQIAIMTFKDYFYCWNTFVENRERTFKAWEGLHENSVRLSRRLRRILLPLDEVDDLRDAFKILGL

SEQ ID NO: 27 - protein, 198 aa, Pogona vitticeps
MDSLLMKQKKFLYNFKNLRWAKGRHETYLCYVVKRRNSATSCSLDFGYLRNKSGCHVEVLFLRYISTWDLDPRHCYRITWFTSWSPCYDCARHVADFLSAYPNLTLRIFAARLYFCEERNAEPEGLRRLHRAGAQIAIMTFKDYFYCWNTFVENRNKTFKAWEGLHENSVRLARKLRRILLPLYEVDDLRDAFRILGL

SEQ ID NO: 28 - protein, 230 aa, Protobothrops mucrosquamatus
MAEKQEQPPVAQNGEKAEKAETAEKAENAEESAEAEKPKELEELPPFEIVTGERLPACFFNFQFKNVEYSSGRNKTFLCYIIEIQDKESKIIRGYLEDEHAAAHAEDAFFNTILPKCESGLRYNVTWYVSCSPCVGCADRITKALQKNKNLHLSISVGRLFMWEEPDIQAALKKMKTAGCKLRIMKPQDFEYVWQNFVEQEEEEAKAFTPWEDIQENFQFYEEKLAELLH

SEQ ID NO: 29 - protein, 202 aa, Python bivittatus
MFLPPCSLLMKQKEFLYHFKNLRWAKGRHETYLCYVVKQRNSATSCSLDFGFLRNKSGCHVEVLFLRYISTWDLDPRNCYRITWFTSWSPCYDCARHVADFLCAYPNLTLRIFAARLYFCEERNAEPEGLRRLHRAGVQIAIMTFKDYFYCWNTFVENRKKTFKAWEGLHENSVRLARKLRRILLPLYEVDDLRDAFRILGL

SEQ ID NO: 30 - protein, 175 aa, Rhincodon typus
MPSAFFKFHFKNVEYSSGRHKTFLCYSVEKQGKECERSEGYVEDEHAGTHAEKAFFTDIFTECDGSLRYSIIWYVSSSPCTVCAAKIAEYLSKCPNVELTIFMARLFMWEEAEIHQGLRQLQAAGCRLRIMKGPEYNYCWNTYVDNEGEAFTPWEDLQDSCTFYEEKLHDILQHA

SEQ ID NO: 31 - protein, 195 aa, Rousettus aegyptiacus
MAAGPAPEARPLMDEQTFLDNFNNLVNPHETYLCHYVERQEGDLWIPVVEFKDFLRNQGADVVLHRWQRRHAEIIFLERMASWRLDKELRYRVTCYISWSPCEDCAYKLAAFLRENTHVDLRIFAARIYDYLPGYEAGLRALRAAGAEVAMMPTDEFERCWESFVDHQGRPFPPWDDLDVKSEELSRRLEGILQN

SEQ ID NO: 32 - protein, 213 aa, Salmo salar
MINKFDSVLLAQKKFIYHYKNMRWAKGRHETYLCFVVKRRGGPNSLSFDFGHLRNRSGCHVELLFLRLLEAGALCPGLWGYGAPDSVGLCYSVTWFCSWSPCSDCSYRLAQFLSQTPNLRLRIYVSRLYFCDPEDSSAREGLRMLQRAGVQITVMNYEDYFYCWQTFVACRQRVFKAWDGLHQNSVQLARKLNDILQPGEAEDWGDAFELLGL

SEQ ID NO: 33 - protein, 295 aa, Salvelinus alpinus
MADKKGAAASSKLVLRKKERTTKTAVTVEVKKEVKKEVKTEMKTEMKREVKREVKSSLVKKEEKVLAVGEKVEGNGEVPMEEGATANEDVANGEAATANANGANGEYEPIELPPWEIIEGDRIDPFQFKFQFKNVEYSSGRNKTFLCYLVDKGKADDGLMRGYLEDEHSGAHAEQAFFLQTLPDYDPAVKYTITWYMSSSPCAVCAAKIAEALQARKSIKMTLFSARLFEWEDQDIQAGLNALSQAGCKLRMMKPMDFTYVWDTFVENDNLTFTPWEDCQDNYEYYHEKLADIMQ

SEQ ID NO: 34 - protein, 201 aa, Xenopus laevis
MTMDSMLLKRNKFIYHYKNLRWARGRHETYLCYIVKRRYSSVSCALDFGYLRNRNGCHAEMLFLRYLSIWVGHDPHRNYRVTWFSSWSPCYDCAKRTLEFLKGHPNFSLRIFSARLYFCEERNAEPEGLRKLQKAGVRLSVMSYKDYFYCWNTFVETRESGFEAWDGLHENSVRLARKLRRILQPPYDMEDLREVFVLLGL

SEQ ID NO: 35 - protein, 235 aa, Xenopus laevis
MAQNKSQSSKDPINNVEEAEKAEKAENGEGNSKKKEQEELPPFEIVEGSRIPASSFMFQFKNVEYSSGRNKTILCYTVERPEGQIFHGYLEDEHVSAHAEDAFFTSVLPQFLTSGSVTVTCYVSSSPCVNCAASIAQCLRRYKTLRIQLAVARLFQWEEPETRRSLKGLRSAGCPVRMMRGADYIYVWKNFVEPDITLDEEGKEVVKDEHQDFIPWEDLEENSRYYEEKLSEILR

SEQ ID NO: 36 - protein, 201 aa, Xenopus tropicalis
MTLDSLLLKRNKFLYHYKNLRWARGRHETYLCYIVKRRYSSVSCALDFGYLRNRNGCHAEMLFLRYLSVWVGHDPHRAYRVTWFSSWSPCYDCAKRTLEFLKGHPNFSLRIFSARLYFCEERNAEPEGLRKLQKAGVRLAVMSYKDYFYCWNTFVESRERRFEAWDGLHENSVRLARKLRRILQPPYDMEDLRDVFELLGL

SEQ ID NO: 37 - protein, 185 aa, Bison bison
MDEYTFTENFNNQGRPSKTYLCYEVERLDGDATIPLDEYKGFVRNKGLDQPEKPCHAELYFLGKIRSWNLDRNQHYRLTCFISWSPCYDCAQKLTTFLKENRHISLHILASRIYTNNHFGCHQSGLCELQAAGARITIMTFEDFKHCWETFVDHKGKPFQPWEGLNVKSQALCAELQAILKTQQN

SEQ ID NO: 38 - protein, 194 aa, Camelus dromedarius
MEASTAPRARCLMDEHTFTHNFSNHKRTHKTYLCYEVEILHGDSGIPPDEDKGFLCNKGTDQPGLPCHVELYLLGRIRSWKLDRKLHCRLTCFISWTPCGTCARELAEFLKENSHVSLCIFASRIYSLNDYEAGLRTLQEAGAQIAIMTFKEFRHCWETFVDHQGRPFQPWDELDINSQGLSKELQAILQTQQN

SEQ ID NO: 39 - protein, 338 aa, Castor canadensis
MIPSLHQYYSRTCLHAEQCFLYWFHEHMLDPAEDYQVTWYISWSPCSTCADEMASFLATHRNVSLTIFSARLYYFWNSAYQNGLRKLYQEGAQVKVMTFQEFEDCWENFVYNKGESFMPWKGLSRNNRFLKNELKKILGSPMSLLKEDIFLYQFNNQQQVQKPYFRRRTYLCYQLEQPNGSRPQWPAKGCLQNKKGHHAEIRFIKRIHSMGLEQDQDYQITCYITWSPCLACACALAELKNHFPRLTLRIFASRLYFHWIRKFQMGLQHLYKSGVLVAVMSLPEFTDCWEKFVNHRQVFFTPWDKLEEHSRSIQRRLRRILQSWDVDDLTDDFRNLRL

SEQ ID NO: 40 - protein, 190 aa, Chlorocebus sabaeus
MNPQIRNPMKAMYPGTFYFQFKNLREANNRNETWLCFTVEGIRHHLTVSWKTGVFRNQVDLETHCHAEKCFLSWFCKNILSPNTDYQVTWYTSWSPCPECAREVAEFLARHNNVMLTIYTARLYYSQCPNYQQGLRSLSEKGVSVEIMDYEDFKYCWQNFVYDDGEPFKPWKGLKTSFRFLKRCLREILQ

SEQ ID NO: 41 - protein, 218 aa, Columba livia
MSCGSSRDQTVLFPFALTSPRATPLPATHRAPVCWNRNRKEGSTKGRKQCWTVHGERRGHAQEKAVRWGDTGRPWIHWIRKDHYHAEVYFLEKIFKMRRSKNFVNCSITWYLSWSPCVSCCCEILNFLEKHSNVNIDIYVAQLYKIQNSEVCEGLKKLVSLKKVTIAVMEIKDYTYCWKTFIQGDADDDSWTVGFQSAITKNRLKLKDVFEDLPFIQS

SEQ ID NO: 42 - protein, 241 aa, Dasypus novemcinctus
MEGSAEPKPSDLLSPEDFTFHYENLKYAHERRKTYLCYHLERLDGDRRVMLDSAVIQNQGSRHVEECFLFSENVLEKLKAPGSYSVTWYLSWSPCARCAQAVIRFLQQHPNVRLRILVARLYFHETRANRAALRALRRREAPIEIMSPEGQLPWGHGGAGQAARPRLCLLLETLRGPPRGGLPALGGYGRKRGPPGRGAERHLAVQVKRARKIGNQEMDLEPDKKSTWMSIPPRWLLEDTP

SEQ ID NO: 43 - protein, 194 aa, Delphinapterus leucas
MEASTAPWTRCLLDENTFNENFMNQINPRKTYLCYKVEILDGDTRVLLDEKKGFVQNKGANEPGKPRHAESYFLDRIRSWNLDRGLHYRLTCFISWTPCHSCAQELATFLGENSHLSLHIFASRIYSLPGYEAGLRKLQAAGAQIAIMTSKEFEHCWENFVDHQDRPFQPWDGLEVVSQHLCNSLQTILQMQEN

SEQ ID NO: 44 - protein, 208 aa, Desmodus rotundus
MARKKFSRKEGYIPKPWPRNPMEKLYADYFNFHFTNWPTPKGRNGCYICYEVERRLHNSPISLVRGVFENEFYPKEPVHAELCFLNWFKEEFLEKTLEPGGIYCVTWYTSWSPCVECARQVAEFLNTHKYVRLRIVSCRLYHSNKPEHQQGLRNLAGAEATLAVMSPEDFHYCWDNFVDNRESDFTYWRGIRRNYYGLHERLRDILSG

SEQ ID NO: 45 - protein, 214 aa, Echinops telfairi
MSTQIRTACGHPPGGHQQRKHAFLKLVQSLISPRDFLFQYENLQFADGRNMTYLCYCVEEKNGDTPMLLHIDVLQNEVAGSHVEILFLSFMRRFLASIDQSLQEARESQVTWYISWSPCADCANTVATFLRERNNVHLRLFASRLYYRDVPEYQQGLRALKEAGAQVEIMSSLEFSDCWDHFVDHQGECFEPWEEMDENVEIFSQKLQDILETR

SEQ ID NO: 46 - protein, 229 aa, Echinops telfairi
MDHRIFIYHFKNDQSVRGRHMTYLCYEVQLFDVRFRVPLGMIRGVLHNQHHPDPQRRCHAELCFLIRMSSLRLYPGQWCQVTWFLSWSPCPRCASKVSDFLRSNRHVSLRIFAARLHTYHAGYEEGLRSLRDAGAQLSIMTAKEFEYCWRVFVDNHSWPFRPWNMLEKSSQAISERLQSILREKISFFHQPTCYQPSFGVFSPGRFPCPPQTASSWFPAPPRGPQHHPF

SEQ ID NO: 47 - protein, 195 aa, Elephantulus edwardii
MEPTPGPYSQCVDTCPSSPLSLALAGQSDLIREDYSEKSKAKLGDEDSRNIDFNFHFENLQKAFERNMTYLCYRLERLDSSGPVLLDAAVLENVPGTHVEMLFLNSRNIQELKQDKKYRVTWYISWSPCAKCAQKVSKFLEAHSNVSLHIFASHLYYWDKPETRQGLRDLQQAEAQIQVMSSLGELAHWMAAGRI

SEQ ID NO: 48 - protein, 293 aa, Eptesicus fuscus
MFGGGGSFITHFCIEMIGGGGGRVLLKQQSRALRERTGAPPSPQAPPPSPCPPSLPRLPTWRQQEMLKGRGCWEAGIPGHLIGETGGNPAFSLFSPAPRNPMRWLSLDYFYFHFTNWPNPKGRNGCYLCYQVKRTQEGLSWDVGTGVFENEFYPKKPVHTEICFLNWFKTQQAFLAQNLSREENYHVTWFMSWSPCFQCARQVVAFLRDHKHVQLSIFAARLYYSRSPQYQQGLRSLQGAGAQVAIMTPADFAFCWDIFVDDPLRPFRYWKGIYINSCSLSKSLEDILRNREN

SEQ ID NO: 49 - protein, 230 aa, Equus caballus
MDPQAPTQRGGLGQAYQGGDYVQAPGNGNTQHLLSEDVFKKQFGNQRRVTKPYYRRKTYVCYQLKLLRGPTIAKGYFRNKKKRHAEIRFIDKINSLGLDQDQSYEITCYVTWSPCATCACKLIKFTRKFPNLSLRIFVSRLYYHWFRQNQQGLRQLWASSIPVVVMGYQEFADCWENFADNRGNPFQSWEKLTEYSKGIKRRLQKILEPLNLNGLEDAMGNLKLGSVDLG

SEQ ID NO: 50 - protein, 230 aa, Equus caballus
MEASAAPMARRLMDEDTFTENFKNVNWPRKTYLCYEVELPDGDSRVPPGWDKGFLRNKPIHMPGPPRDAEMRFLDLISSWKLDQKLRYRVTCFISWSPCADCAQRLAEFLRENSHVSLRIFASRIFTKGDYKAGLRTLQAAEAQIAIMASEEFEHCWETFVDHQGRPFQPWDELDPKSRYLSEQLLHILQPTAPPSLPPLYFPPSLPPLYFPPSHPVPGPYFRSLRDSSG

SEQ ID NO: 51 - protein, 194 aa, Globicephala melas
MEANPSAGTSCLLDENTFTENFMNQMNPRKTYLCYKVEILDGDARIPLDEKKGFVRNKGANEPGEPCHAEHYFLDRICSWNLDRELHYRLTCFISWTPCDTCAQKLADFLGKNSHVSLHIFASRIYSRLGYVAGLRTLQAAGAQIAIMTSNEFEHCWENFVDHQGKPFQPWDGLNVVSQRLCNNLQAILQTQEN

SEQ ID NO: 52 - protein, 190 aa, Hylobates moloch
MNPQIRNPMERMYRPTFYYNFENEPILCGRSYTWLCYEVKIRKDPSKLPWDTGVFRGQMYFQPEYHAEMCFLSWFCGNQLPAYKRFQITWFVSWTPCPDCVAKVAEFLAEHPNVTLTISAARLYYYWEKDWQRALCRLSQAGARVKIMDYEEFEYCWENFVYNEGEPFMPWYKFDANYAFLHHTLKEILR

SEQ ID NO: 53 - protein, 218 aa, Lagenorhynchus obliquidens
MWGAEGKSVRGGEERWAVPEGAPCLWASPAFDRSCLLDENTFTENFMNQMSPRKTYLCYKVEILDGDARIPLDEKKGFVRNKGANEPGGPRHAESYFLDRIRSWNLDRDLHYRLTCFISWTPCDTCAQELADFLGKNSHVSLHIFASRIYSRFGYEDGLRTLQAAGAQIAIMASNEFEHCWENFVDHQGKPFQPWDGLEVVSQRLCNNLQAILQTQEN

SEQ ID NO: 54 - protein, 237 aa, Leptonychotes weddellii
MSFEDYEYCWDNFVDHRGMRFQSRNLLRDNDLLATELENILRTTMNPLQKEIFFYQFGNQPRAPKPYHRRTTYLCYQLKPHEGPIIARVCLQNKKKRHAEIRFIDNIKALRLDTSQRFEITCYVTWSPCPTCAKELVAFVRDHRHISLRLFASRLYFHWLRENKKGLKRLHASRIPVAVMSSLEFEDCWKEFVDNQGKPFQPWNKLEQYSESITRRLRKILEPQNDLENEFRNLQLQ

SEQ ID NO: 55 - protein, 155 aa, Lontra canadensis
MATSPTLPPALQPRGSPSLLRLSQVYPQAPCHAEACFLSWFRDQNLPPEEHYRVTWFLSWSPCPTCAEDVVEFLEEYRNVTLSIYTARLYYFWDPDFQDGLHKLWCAGVHLDIMSFEDYEYCWETFVDHKGMRFQSWDLLRDNDLLAAELDNILG

SEQ ID NO: 56 - protein, 184 aa, Loxodonta africana
MAKGIFLPDFTNDPSQFRGLKRTYLCYEVELLDGNSWVPLDNGRDFLSNQPRRHAELCFLDRVSSWHLDPTKRYKFTWFLSWSPCRNCAQEVAAFLGGNSHVSLSIFAARIYDCYPGYEEGLRSLQGAGAHVAIMTSTEFERCWRTFVDNGGCLFVSWNGLNESSQMLKRRLESILQGQLRRHP

SEQ ID NO: 57 - protein, 211 aa, Mandrillus leucophaeus
MALLTAETFSLQFNNKRRVNKPHYPRKALLCYQLTPQNGSTPTRGHLKNKKKHHAEIHFINKIKSMGLDETQCYQVTCYLTWSPCPSCARELVDFIKAHRHLNLRIFASRLYYHWRPHYQEGLLLLCGSQVPVEVMGLPEFSDCWENFVDHKEPPSFNPSEKLEELDKNSQAIKRRLERIKQSRSVDVLENGLRSLQLGPVTPSSSIRNSR

SEQ ID NO: 58 - protein, 190 aa, Mandrillus leucophaeus
MNPQIRNPMKAMDPGTFYFQFKNLWEANNRNETWLCFTVQVIKHHSTVSWERGVFQNQVDLETHCHAERCFLSWFCEDILSPNTDYRVTWYTSWSPCPECAREVAKFLARHNNVMLTIYTARLYYSQYPNYQQGLRRLSEEGVSVEIMDYEDFKYCWENFVYDDGKPFKPWKGLKTNFRFLKRRLQEILE

SEQ ID NO: 59 - protein, 243 aa, Monodon monoceros
MEASTAPWTRCLLDENTFTENFMNQINPRKTYLCYKVEILDGDTRVPLDEKKGFVHNKVTDPACPQQAGPPYCGTLRVEGCQLHTGCPTSSLTPGPCRCYCCSAWNQGANEPGKPCHAESYFLDLIRSWNLDRGLHYRLTCFISWTPCHSCAQKLATFLGENSHLSLHIFASRIYSLPGYKAGLRKLQAAGAQIAIMTSKEFEHCWENFVDHQDRPFQPWDGLEVESQHLCNNLQVILQMQEN

SEQ ID NO: 60 - protein, 243 aa, Neophocaena asiaeorientalis
MEASTAPWTSCLLDENTFTENFMNRLRPRKTYLCYKVEILDGDARVPLDEKKGFVRNKVTDPACPQQAGPPYCGTLRVEGCQLHTGCPTSSLTPGPCRCYRCSAWNQGANEPGMPRHAECYFLDRIRSWNLDRGLHYRLTCFISWTPCHSCAQELATFLGENSHVSLHIFASRIYRRPGYEAGILTLRAAGAQIAIMTSKEFQHCWENFVDHQERPFRPWVGLEVESQHQCNELQAILQTQAN

SEQ ID NO: 61 - protein, 194 aa, Orcinus orca
MEASPSPGTSCLLDENTFTENFMNQMSPRKTYLCYKVEILDGDARIPLDEKKGFVRNKGANEPGEPCHAEHYFLDRIRSWNLDRELHYRLTCFISWTPCDTCAQRLADFLGKNSHVSLHIFASRIYSLSDYEAGLRTLQAAGAQIAIMTSNEFEHCWQNFVDHQGKPFQPWDGLEVVSQHLCNNLQAILQTQEN

SEQ ID NO: 62 - protein, 211 aa, Papio anubis
MALLTAETFSLQFNNKRRVNKPYYPRKALLCYQLTPQNGSTPTRGHLKNKKKNHAEIRFINKIKSMGLDETQCYQVTCYLTWSPCPSCAGKLVDFIKAYHHLNLSIFASRLYYHWRPNYQEGLLLLCGSQVPVEVMGLPEFTDCWENFVDHEEPPSFNPSEKLEELDKNSRAIKRRLERIKQSRSVDVLENGLRSLQLGPVTPSSSTHNSR

SEQ ID NO: 63 - protein, 267 aa, Phocoena sinus
MWGAEGKGVRGGEERWAVPEGAPRLRASPAFDRSCLLDENTFTENFMNRIRPRKTYLCYKVEILDGDARVPLDEKKGFVRNKVTDPACPQQAGPPYRGTLRVEGCQLHTGCPTSSLTPGPCHCYRCSAWNQGANEPGMPRHAECYFLDRIRSWNLDRGLHYRLTCFISWTPCHSCAQELATFLGENSHLSLHIFASRIYRRPGYEAGILTLQAAGAQIAIMTSKEFQHCWENFVDHQERPFRPWVGLEVESQHQCNELQAILQTQAN

SEQ ID NO: 64 - protein, 280 aa, Physeter catodon
MEAGTAPRSRCLLDENTFTENFKNQINTRKTYLCYEVEIVDGDARVPLDEIKGFVHNKGANQPGEPCHAELYFLDRIHSWTLDRRLHYRLTCFISWTPCHTCAQELATFLGENSHVSLHIFASRMYRRFDYEAGLRRLQAAGAQIAIMTSKEFEHCWETFVDHQGRPFQPWDGLEVESQHLCKKLQEILQVRAPSCCFPGLPPSSPLLSSPCAFFLLSEPPLGDLLPPGAPAPSFPSFLMRSLYFLTCCVPPSFPIFVTLPRPPLGFCFILQMSHSLLPS

SEQ ID NO: 65 - protein, 198 aa, Pteropus vampyrus
MAAGPAPEARSLMDEQTFLDNFNNLKYPRKTYLCYKVERLVGENSIPVDEYKGFLRNESSPRRWERRHAEPIFLERMASWNLDTELRYRITVFISWSPCPECADELVKFLRENRHVKLRIFAARIYDWYQGYEAGLRALKAAGAEVAMMTFQEFEYCWKNFVDHQQDEDKPFPSWDNLVVRSEELSQRLAGILQNREE

SEQ ID NO: 66 - protein, 265 aa, Trichechus manatus
MDTGIFLCYFSNDPRAREQYRTYLCYEVELLDGNSWVLLYKSKGFLPKQLDPAQCHRVTWFMSRSPCPDCAREVTAFLGKNSHVHLHIFAASIYDYSGGYEEQLLSLWHAGAKIAIMTPTDLMHQDIFQFNFNNNSLVCGQKQTYLCYEVELLDDNSWVLLEGGRGFLNNQLDPAQCYRVNCFMSRRPCFLCAWEVATFLGRNSHVSLHIFTSCTYEYFPSSECKHYWRTFVDNQGCPFEPWNRLDKNSQTIWKRLQRILQNEYY

SEQ ID NO: 67 - DNA, 186 bp, synthetic construct
  source 1..186: mol_type = other DNA
atggaccctgaaacacattgccacgcagaaagatgttttctctcgtggttctgtggtaatcagttgccagcgtacaagtgcttccaaataacctggtttgtgagttggactccttgcccagattgtgtggcaaagctggcggagtttctctcagaacacccgaatgtgactctgaccatctcagcc

SEQ ID NO: 68 - DNA, 576 bp, synthetic construct
  source 1..576: mol_type = other DNA
atggctgatagtagcgagaagatgcgcgggcagtatatcagccgcgacactttcgagaagaactataagccgattgatggtactaaagaggcacatctgttgtgtgaaattaaatggggcaaatacggcaagccctggctccattggtgtcagaaccaacggatgaacatccatgcggaagactactttatgaataacattttcaaggcgaaaaagcacccagtccactgctatgtgacctggtacctctcatggtcgccctgtgccgattgcgcatctaagatagtgaagttcttggaagagcgcccatatctcaagttgacgatctatgtcgcgcaactctattaccacaccgaagaagaaaatcgcaagggactgcggcttctgaggtcgaaaaaggtcataatacgggtcatggatatatctgattacaactattgctggaaagtttttgtgagtaatcagaatggaaacgaagactattggccgcttcagtttgacccgtgggtgaaggaaaattattcccgcctcttggatatattttgggagtcaaaatgccgctcccctaacccgtgg

SEQ ID NO: 69 - DNA, 684 bp, synthetic construct
  source 1..684: mol_type = other DNA
atggccgagaaacaggaaaaccctgaaaacacacaaaatggtgaaccagagactgaaaacgccgaactgaccgaagaggaaaaagagaaggagaaggaacgcaagaagaaagagttggaagaacttcccccttttgaaatagttagcggcgagaggctgcccgcattctttttcaaatttcagtttaaaaatgtggagtattcatcaggccggaataagactttcctttgttatattgtggaaacgcagggtaaagaatcaggcacaatccgggggtatctcgaggacgagcacgcggcggcacacgcggaagatgccttcttcaacaccattttgcccgaatgcgaaagctcattgcgctataatgtgacatggtacgtgagttcatccccttgtgtcgcatgcgcggagaggatcgcagaaatccttaagaaaaataaaaacctcagactcacaattctggtttccagattgttcatgtgggaggaaccagagatgcaagcagtcttgaagaaaatgaaagcggctgggtgcaaattgcggatcatgaaaccgcaggactttgagtacgtgtggcagaattttgtggagcaggaagagggcgaatcgaaggatttcgtgccttgggaggatatacaggaaaattttctttactacgaggaaaaacttgcggaggttcttcac

SEQ ID NO: 70 - DNA, 594 bp, synthetic construct
  source 1..594: mol_type = other DNA
atggactcccttcttatgaagcagaaaaaatttctgtataattttaagaaccttaggtgggcgaaagggcgccatgaaacatatatgtgctatgttgttaaaagaagggattcagccacctcctgttcccttgatttcgggtacttgcggaataagtccggctgtcatgtggaggtgcttttccttcgctatatctcggcatgggaccttgatccaggacgctgctatcgggtcacctggtttaccagttggtcgccttgttatgattgcgcgcggcacgtcgccgacttccttcgggcgtacccgaacctgactctgcggatcttcgttgcccggctctatttttgcgaaggcagaaatgcagagccggaggggctccgccgcttgcatagggccggtgctcatattgctattatgacatttaaagactacttttattgttggaacaccttcgtggaaaacagggagcgcacattcaaagcgtgggagggcctgcatgaaaacagtgtccacctgactaggaaactcagaagaatccttcttccactttatgaggtggacgatctgcgggacgcctttaggaccctgggactg

SEQ ID NO: 71 - DNA, 522 bp, synthetic construct
  source 1..522: mol_type = other DNA
atgcaaggctggaaacttgaggcggaggagttcctttcgaactttggaccctcagagcaccctgaagttacctacctcctctacgagatccaatggggtcgcagtcggaggacttggaagaattggtgtcggaacatccacccggaacatgcggagataattttcttggaagacgaggaagtccatgacagacctgacattccttgcaacatcacctggttcgtcccctggacaccttgcggagcatgtgcttatgagataatcgcttttcttgaggaaaggcccaatgttaacctcgaaattcttgctgcgcaggtgtataaaccgtatgaccataggaatagaaaagggctgagggatctggcggacgaagctcaaatttctatcatggagtttagcgattatcaatactgctgggaaaaattcgtggatcatcaaggcatgaacctgagggcgggcagagggttctcaaaccaacggagaatcgattctcggcaactggattccataatcaacccgttc

SEQ ID NO: 72 - DNA, 582 bp, synthetic construct
  source 1..582: mol_type = other DNA
atggaagcgtccaccgcgccaggtacaaggtgcctgctcgacgaaaatacgtttaccgagaatttcatgaatcagataagtgttaggaagacatacctgtgttacaaagttgagattcttgatggggacgcttgggtccctttggatgaatataagggctttgtgcataataaaggagccaatcaacctgggcagccctgtcacgctgaactctacttccttgatcgcatcaggtcctggaacttggaccaagagttgcactatcggcttacctgctttatcagttggactccctgccatacgtgcgcccaaaatcttgccacgtttttgggagaaaatagtcatgtctctttgcgcatctttgcgtcacgcatttattctctcgctggttacgaggcagggctcagaactctccaagcggcaggagcacaaatagcgattatgacctctaaagaatttgagcattgctgggagacatttgtggatcatcaaggaaggcccttccaaccgtgggacgagcttgaggtcgtgtcgcagcacctgtgtgaaaagctgcaggctattcttcaaacacaacaaaac

SEQ ID NO: 73 - DNA, 738 bp, synthetic construct
  source 1..738: mol_type = other DNA
atggcagataagtcccggctcgccatgaggagaaaggataagaaagaggaactggagccagtgaagaatgtgaaggaggttaaagaggtcaaagaggaggagggggagaaacccgcggaaaatggtgaggcaccacaagagaatggcgccgcggcggaaaatggcgaaccccttctggaacctatggagctgcctcctttcgaaataattacaggagatcggatagaccccttcgttttcaaattccagtttaagaatgtcgaatattcgagcggccggaacaaaacctttttgtgctatttggttgatcggggttcggtcgagggacttcttagaggttacctcgaggacgagcacactggtgctcatgccgaggaggcattctttactcaatgtttgcccgactatgatcctgccgtgaagtacactatcacctggtacgtcagctcaagtccatgtgccgcatgcgctgcgaaaattgcagaagtcttgaaaacgagaaaaactgtgaaactgtccatctttgcgagtagactctttgagtgggaggagcccgagattcaagcaggcctcaagacccttcacgcggcaggttgcaagctgaggatgatgaaacctctggacttctcttacacttgggatacattcgtcgagggcgatgaggaggcctttaacctttgggaggattgcaaagataattatgagtattatcaggacaagctcgcagacattttgcag

SEQ ID NO: 74 - DNA, 660 bp, synthetic construct
  source 1..660: mol_type = other DNA
atggccgagaatcaagagaagccgtcggatacacataacggtgaagaacaacctgaaaatgcggaaaaaccagaggagaaaaagaaggaactcgaaaagttgccccccttcgaaattgttcgcggagaaaggtttcctgctttctttttcaaatttcaatttagaaatgtggaatactcttctggtcggaacaaaactttcctctgttatgtggtggagatccaaggaaaggagccaggtagtttccaaggctacctcgaggatgaacacgctgcagcgcatgcggaggacgcttttttcaataacattttgccaacgtgtgatcccaaccttagatacaacgtcacatggtatgtttcatcgagcccgtgtgtgccatgtgcggatcggattgctcaagtgctgcaaaaaaataaaaatcttaggttgtcaattttggtcagtaggctctttatgtgggaggaaccagagatgcaagcagccctgaaaaagttgaaagcaagtggctgcaaacttaaaattatgaagccacaagacttcgaatatatatggcaaaacttcgcagaacaagaggaaggcgaagctaaggcttttgaaccttgggaagatattcaggagaatttcctctactacgaagaaaagttgtctgaggtccttcat

SEQ ID NO: 75 - DNA, 567 bp, synthetic construct
  source 1..567: mol_type = other DNA
atgttctatgaaataaagtggtccaatagtaaaaggagctggcaatcctgctgtcattccacacggacggaacacgcagagatacatttcattgaggacgtttttcaggaacaaagaagtgacccgagtgtccattattcgataacgtggtacatgagttggagtccctgcgctgattgctgcaaggagattagggactttctgaaggaacagccaaatgttaacctcgtcatatacgtggcccgcctgtactggcataaagaggaaactaatcgccagggtcttcgctccctggtgaacataggggtcagtattagagttatggaccttccagcttactcatattgctggagaacctttgtcaatgacgaggacaaggacgaagacgactactggccccgccactttgctccatggattatgctttatagccttgagttgcaaagtatcctgcagaacattccctcctgcctcgaaatctcaactgatgagaatcagactccggtgttctcgctgtgcgtcgaagatgaccagcaaaaacgcgctctcacctcggcaaatccacgc

SEQ ID NO: 76 - DNA, 594 bp, synthetic construct
  source 1..594: mol_type = other DNA
atggattcccttctgatgaagcagaaaaagtttctttacaattttaggaacctgagacgcgccaaaggtcgccacgagacttacctctgctacgtcgttaaacgccgggacagcgctacttcgtgtagccttgactttggctatctcagaaataaatctgggtgtcatgttgagatgatcttcctccgctacatatctgcttgggatctcgatcccggaagatgctaccgggtcacttggttcactagttggagtccttgttacgattgcgcaaggcatgtcgtcgattttctcagagcataccctaacctcactcttagaattttcgtcgccaggctctatttttgcgagggaaggaacgcggagcctgagggcctgaggcgcctccatcgggctggagcacatattgctataatgacatttaaggactacttctattgctggaatacatttgtggaaaatagggagcgcacttttaaggcctgggagggattgcacgaaaactcggtgcatctgacccgcaagctcagacgcatccttctcccgctctatgaggtcgatgatcttcgggacgctttccgcaccttgggcttg

SEQ ID NO: 77 - DNA, 684 bp, synthetic construct
  source 1..684: mol_type = other DNA
atggctgaaaagcaagagaatccggaaaacacccagaatggtgagccagaaccagagaatgcagagcttacagaagaggaaaaagagaaggaaaaggagagaaaaaagaaagaattggaggaactccccccattcgagatagtgtccggcgagcggctccccgcgtttttctttaaatttcaatttaaaaacgtcgaatacagttccggtcggaacaagactttcttgtgttacatcgtggagacgcaagggaaggaaagtgggacaatcaggggctacttggaagatgagcacgcagccgcccatgcagaggatgccttcttcaatacgatattgccagaatgtgaaagctctctccggtacaatgtcacttggtacgtttcatcctctccttgcgtgccgtgcgcggaaaggatagccgaaatacttaaaaagaataaaaacctcagactgactatcctggtgtctaggctctttatgtgggaagagccggaaatgcaagcggtcctgaagaaagttaaggcagcgggctgtaagttgcggattatgaagccccaagactttgaatatgtttggcagaactttgttgaacaggaagaaggtgagagtaaagatttcgtgccgtgggaagacatacaagagaattttctgtattacgaggagaaactggcagaggtcctgcac

SEQ ID NO: 78 - DNA, 753 bp, synthetic construct
  source 1..753: mol_type = other DNA
atggccgacaagggtagggcagtggcgcggcgcaaggaccgcaaagagccggatgctgcagatcgggcaaagtccggcaaagaagaagaaaacatcccagaaaaaaccgaaaataatgaaggagccccggcggcagaggaaccagctgggggggcagttgcgaacgggcagaacggcgaatttaaggtggaaccgatggaactgccgccctttgagatcatccagggtgacaggattgatgcgtttgcatttaagtttcagttcaagaatgttgaatatagctccgggagaaacaagactttcctgtgctatcttgtggataaagggaaagcatcggacggcctgctcagggggtatcttgaggatgagcacagtggagctcatgccgaagaagccttttttatacaatgcctccccgactatgatcctgctttgaagtacacaatcacttggtacgttagttcttctccgtgtagtgcatgcgccgaaaaaatagctgaagttctgaaggcacgcaaaaacataaaactctcaatttttgcggcgcggctctttgaatgggaggaggcggaaattcaggcaggtatgaagtccttgaacgcggcgggttgtaagttgagagtcatgaaaccacttgacttctcatacatttgggataccttcgttgaaaacgaagaccaacctctgaacctttgggaagactgcaaagacaactacgaatattatcacgagaaactctctgaaatccttcaa

SEQ ID NO: 79 - DNA, 630 bp, synthetic construct
  source 1..630: mol_type = other DNA
atgattagtaagcttgactcggttctgatgacccaaaaaaaattcatcttcaattataagaacgtcagatgggcaaggggacggcacgatacatatctctgctttgtcgtcaagcggcggaccggaccagattctctctctttcgatttcggacacttgcgcaaccgcacgggttgccacgtcgagcttttgtttctgaggcaccttggagctttgtgtcccggtttgagcggctctagtgttgacggtgccagaatgtattacgccgttacttggttttgcagctggtccccatgctccaaatgctcgcagcagttggcgcacttccttagtcaaacgcctaacctccgcctgcgcatatttgtcagcagactttacttctgtgatgaagaagatagtcaggagcgggaaggtcttagacaccttaaaagggcaggggtgcagatctccgtgatgacctacaaggactacttttactgctggcaaacattcgttgcccggcgcgagagatctttcaaagcctgggatgacttgcatcagaatagtgtccggttggttcggaaattgaacaggatacttcagccatgtgagacggaggatcttagagatggctttgcattgctcggactt

SEQ ID NO: 80 - DNA, 630 bp, synthetic construct
  source 1..630: mol_type = other DNA
atgatctgcaagttggattcagtgctgatgacacagaagaagtttatttttcattacaagaatgtgagatgggctaggggtaggcacgaaacttacctctgtttcgtcgtgaagcggaggatcgggccggattcgctgtcgttcgatttcggtcaccttaggaatagatctgggtgccacgttgagttgcttttcctccgccaccttggcgcgctttgccccggtctgtccgcctcgagcgtcgatggagcccggctttgttattcggtcacctggttttgcagctggtcaccatgctcgaagtgcgctcaacaactcgcccattttctgtcgcagacgccgaatctgcggcttagaatattcgtgtcgcgcctctacttctgcgacgaagaagattcagtggaaagggagggtctgaggcatcttaagagggcaggagtgcagattagtgtcatgacatataaggactttttctattgctggcaaacatttgttgcacggagggagcggagctttaaggcttgggatggtctgcacgagaactcagttcggctggttaggaaattgaatcggattctccaaccatgtgagacggaagatctcagagacgtttttgctcttttgggactg

SEQ ID NO: 81 - DNA, 747 bp, synthetic construct
  source 1..747: mol_type = other DNA
atggctgatcgcaaaggatcttctggagtgagttccaggctttctgttcggagaaaagagaaggcggaaaatgacgctaagaaagaggagaagtctccgaccgaggcggaaaaacctgaggtcaatggaaaggaagtcccgatggagaatggcgaagccggggccgcagccgccgatggcgagaaaccggagccgattgaactgcctccctttgaaacaatcacgggagatcgcatggatccattctttttcaagttccagtttaaaaacgtcgaatattctagtggtcggaacaagacctttttgtgctacctcgtggatcatggtggagagggactgatgagaggttacatagaggatgagcacgcgggagggcacgcagaagaggctttcttccaacaaattcttaccaactacgaccctgcgtgcaagtacacaattacctggtatatgtcaagctctccctgcgcgaattgtgccacaaaactggccgagatactccggtctaggaaaaatatacgcctggcaatcttctcttccaggcttttcgagtgggaagagccagaaatccaggcgggactgaaattgctcgcaagtgtgggctgcaaactccgcatgatgaagccgctcgacttcacatatacgtgggacaccttcgttgaaagtgatgagcagcaatttacgccatgggaagactgccaggaaaactatgagtactatcaagataagcttgcagatatactccag

SEQ ID NO: 82 - DNA, 570 bp, synthetic construct
  source 1..570: mol_type = other DNA
atggaaggtagcgcggaaccgaagccctcggacctcctctcacccgaagatttcacattccattatgaaaacttgaagtatgctcacgaaaggaggaagacttatctctgttatcacctcgaacggttggacggcgacaggagggttatgcttgacagtgcagtcattcaaaatcagggctcacggcacgttgaggaatgtttcctttttagtgagaacgtgctggagaaacttaaggctcctggaagttactctgtgacgtggtatctgtcttggagcccctgtgcgaggtgtgcacaggctgtgatacgcttcctgcagcaacacccgaacgtcaggttgcggatacttgtcgctaggctgtactttcacgagacgcgggccaatagagcagcccttagagcactgagaagaagggaagctcccatcgagatcatgtcgcctgaggattttgcatactgttggaaacatttcgttgaccataggggcgaagacttccaaccttgggaggacatggacgctaatgtcgataggctcgcggaagagttgagtgggatctggctcagtaag

SEQ ID NO: 83 - DNA, 630 bp, synthetic construct
  source 1..630: mol_type = other DNA
atgatcactaaactggatagtgttttgatgactcaaaaaaagttcatataccattacaagaacttgaggtgggcaaggggccggtgtgaaacgtatttgtgctttgtggtcaaacgccggataggtccaaatagtctctcgttcgattttgggcacttgagaaatcgctcgggttgtcacgttgagctgttgtttttgcgccaccttggcgcgctttgccccgggctttggggaagtggtcctggtgaagttaaagcgttttatagcgtcacatggttttgctcgtggtcgccttgcagtgattgctcacggcgggttgcaaactttctgcttcgcactccgaacttgcggcttcgcatcttcgtggcccgcctttatttttgcgacttggaagattctctcgaaagggagggcctcagaacattgaagcgggcaggcgttcatctttcggtgatggcgtataaagattactattactgttggcaaacttttgtcgcaagaagacagcgcgcattcaaggcgtgggacggtctgctccagaacagtgtcaggttggctaggaagctcaaccgcatactccagccttgtgagacggaggacctgagagatgcgtttgctttgttgggtttg

SEQ ID NO: 84 - DNA, 597 bp, synthetic construct
  source 1..597: mol_type = other DNA
atggcggtgggggagaaaaagggacttctgggaacttcgcagagatggaaaatcgaactgaaatcctttcaagaaaattatatgccaagcacctggcccaaagtcactcatctcctttatgagattaggtggggcaaagggtctaaggtttggaggaaccggtgcagcaacacattgacacagcacgcggaggtgaattgtctcgaaaacgcattcgggaagctccagtttaaccatccggtcccgtgtcatattacttggttccttagctggtcaccatgcgggcgctgctgtcgccgcatactgcaattcttgcaggcccatcctcatgtcacccttgttatcaaggctgctcaactgttcaaacatatggatgagcgcaacagacaaggcttgcgcgatctggtgcagaacggtgtccatgtgcaggttatggacttgcctgactacaggtattgttggagaacttttgtgactcatccgcatgaaggtgaatcagatttttggccctggttctttcctttgtggatcactttttacaaccaagagcttcaacatatactgcttgtctctcgctacttcggcaaaaacgttggc

SEQ ID NO: 85 - DNA, 624 bp, synthetic construct
  source 1..624: mol_type = other DNA
atgcttccgcatttcctcccaccgctgactcacagtttgttgatgaaacgccggaaattcctgtaccacttcaagaatgtccggtgggctaaaggtaggcatgagacttacctgtgttatgtggtcaaaaggcgcgattcagcaacctctttctccctcgattttgggcacttgcggaataaagctggatgtcacgtcgaactccttttcctccggtacatatccgattgggatctcgatccgggaaggtgttatcgggttacgtggttcacgtcgtggagtccgtgctatgactgcgcaaggcatgtggctgacttcctcaggggatatcccaatctttcccttcgcatctttaccgctcggttgtatttctgtgataaggagcggaaagcagagccagagggcctgaggagacttcaccgggcgggcgtccagatagccataatgaccttcaaggattatttctactgttggaatacgttcgttgaaaaccacgaaaggacatttaaggcttgggaaggactgcacgaaaactccgtgaggctgtcacggcagcttaggcggatcttgcttccattgtacgaagtcgatgatctgcgggacgcatttaggacacttggcttg

SEQ ID NO: 86 - DNA, 438 bp, synthetic construct
  source 1..438: mol_type = other DNA
atggatagcttgctcatgcaaaaaaagaaattcttgtaccattacaaaaacgtgaggtgggccagggggcgccatgagacatacctgtgttatatcgtgaaacgccgctataacccggcgagttactctctggactttggtttccttcgcaataaaagcgggtgtcatgtcgagatgctttttctgaggtttctcacaggatggaatatcgaccccactctcccatatagtgtgacttggtttacctcgtggagcccctgttatgactgctcacaacacgtcacacattttttgcgcgtttaccctaaccttcgccttagaatcttcacagctaggctctatttttgtgaagagaacaacgctgaaccggaggggctgcggaatcttcatatggccggtgtgcagctcggtgtgatgacgtttaagggtaaaggtggg

SEQ ID NO: 87 - DNA, 705 bp, synthetic construct
  source 1..705: mol_type = other DNA
atggcgtctgatagagggccttctgctggtgatgcgacctcaaggcggagaattgaaccctgggagttcgaggtcagtttcgacccgagggagctgtgtaaggaaacccggcttctttacgagattaagtggggaaaaagtcaacacgtttggagacactcatcaaaaaataccaccaaacacgtggagcgcaatgtcattgaaaaaatcaccagcgaacggcggttccaaccatcagtctcctgctgcatcatctggttcctgtcgtggagcccttgttgggagtgtagcaaggcgatccgggaattcttgaatcaacacccacgcgtcaccctgattatttatgtggcccgcttgtttcagcacatggacccccaaaatcgccagggtttgagggacctgatccacagcggcgtcacaatccaaatcatgggacccccggaatatgactattgctggcggaatttcgttaattacccaccgggtgaggaggcgcattggccgcggtatcccccgcttcttcttaagttgtatgtcttggagttgcactgcatcattctcgggctgcctccatgcttgaacatctccagatgccaaaatcagctcacattgttccggcttattccccagaactgtcattaccaaatgattcctccgcatatactccttcatacgggactcattcaactgcctatgacctggcgg

SEQ ID NO: 88 - DNA, 582 bp, synthetic construct
  source 1..582: mol_type = other DNA
atggattccctcctcctcaaacgcaacaaattcttgtaccattacaagaacatgcggtgggcacggggcagacacgagacttatctgtgctatattgtcaagagacgctattcgagcgtgtcaactagtatggactttggctacctgcggaacaggtccggttgtcacgctgagatacttttcctccggtatttggctatttggttgggccacgatccgaaccggatgtttagggttacttggttctctagttggagcccgtgtcgggattgctcgcagaggactgcggacttccttgtcaaccaccctaacctgtcgctccggatcttttcagcgaggttgtacttttgtgaagaacatacagcagagccggaaggacttcggaagctccaaagggctggggttcaagttgcctattacttctattgctggaacaacttcgtggaaaacagagagaggaagtttgaggcatgggatggactgcacgagaacttcgtgcggctctcccggaaactgcgccggatcttgcagcctccgtatgacatggaagacctccacgaagcgttcgacctccttggattg

SEQ ID NO: 89 - DNA, 654 bp, synthetic construct
  source 1..654: mol_type = other DNA
atgtggggtgcggaaggcaagggagttcggggcggtgaagaaagatgggcggtcccagaaggggccccgagactgagagcttcacctgcctttgacagatcttgtcttttggacgaaaacacattcaccgaaaattttatgaatcggttgaggcctcgcaaaacttacttgtgctacaaagtcgaaatccttgatggtgacgcccgcgtgcccctggatgagaaaaaaggcttcgtgcggaacaaaggtgctaatgagccaggcatgccgcggcatgccgagtgttatttcctggacagaattcgctcgtggaatttggacagaggcttgcactatagactgacatgttttattagttggacgccctgtcattcatgcgcccaagaattggctacgttcctcggcgagaactctcatgttagcctgcacattttcgcatcgcggatatatcgcagaccaggttacgaagctggtatattgactcttcgcgcggcgggagctcaaatcgccattatgacttccaaggagtttcagcattgttgggaaaattttgtcgatcaccaggaaaggcctttccggccctgggttggcttggaggtcgagtcccagcatcagtgtaacgagcttcaggccattttgcagacacaggcaaat

SEQ ID NO: 90 - DNA, 423 bp, synthetic construct
  source 1..423: mol_type = other DNA
atgaagagacgcaaatttttgtaccatttcaaaaatgtgcgctgggccaaaggtagacatgagacttacctttgctatgtcgttaaaagacgcgattccgcgacaagtttttcgttggattttggccatcttaggaacaaggctggttgtcacgtcgagcttctttttctcaggtatataagcgactgggacctcgatcccggtagatgttatagagtgacctggttcacatcttggagcccctgctacgattgcgctagacacgttgcggatttcctccgcggataccctaacctgtcactccgcatatttacagctcggctctatttttgtgataaggaaagaaaggccgaacctgaaggtctgagaaggctccaccgggcaggggttcagatagcaattatgacgttcaaggcgcccata

SEQ ID NO: 91 - DNA, 828 bp, synthetic construct
  source 1..828: mol_type = other DNA
atgtcggataagaaggaggggaaggctccgggtaaacttccaggcaggaaaaaaatggaaaaaaaggcagataaggcacccgagaaagaaaaggttgaaaaaaaagtcgagcgcaaaatggagaaggacccagataaagcagtggagaaagtgccaactgaaaaggcgactgaaaacgcgccagaggtggccgagggtacggttggaaatggcgcaccatcggaaggagcagtcgcctcaggtggaggtgaggagtttgtggtggaaccgatggagctcccacccttcgaaattatcgttggcgagaaaattaacccattcttcttcaagttccagtttaagaacgttgagtattcgtcaggtcggaacaaaacatttttgtgcttcctcgttgatgcccagaatagtaacggggagggccttcgcggttaccttgaggacgaacataccggcgcacatgctgaggaagcattcttccaccaagtgctcccgcagtatgaccctgcacttcggtatatggtcacctggtatgttagtagctcaccgtgcgcagcatgtgcagctaagttgaccgagatccttaagacgagaaaaactttgcatttgacgatcttcagcagccgccttttcatgtgggaggaacccgagatacaggtggggctcaaagcgttggtcgctgctggctgcaaattgagaatgatgaagccctcagactttatttatacttgggataccttcgttgagaatgaggatcaatcatttacgccgtgggaagattgtcaagagaattacgaatattaccaggagagattggctgacattcttcag

SEQ ID NO: 92 - DNA, 654 bp, synthetic construct
  source 1..654: mol_type = other DNA
atggagctgttgtgtactgtcaactcattggcggctgtgcaactccagtcaaagcgcgtgtgtcgctcgcttctcatgaagcaaaaaaagtttctctataatttcaagaacctccgctgggccaggggaaggcacgagacgtatctgtgttatgtcgttaagaggagagactcggctacttcattctctcttgacttcggttacttgagaaataaaagtgggtgccacgtcgagatgctcttcctgcggtatatcagcgcgtgggacctggatccagggcgctgctatcgcgttacttggttcacatcctggtcgccatgctacgattgcgcccgccacgtcgctgactttcttcgggcctatccgaatctgactcttcgcatattcgcggcccggctttacttttgtgaggatcgcaatgcagaacctgaggggttgagaagacttcatagagctggggttcagatagcgattatgaccttcaaagattatttctactgctggaatacgtttgtggagaatagggaacggacgttcaaagcatgggagggactgcatgagaatagcgttcggctttcccgccgcctccgccgcatcttgctgcccctcgatgaagttgacgacctgagggacgcatttaaaatcctcgggctc

SEQ ID NO: 93 - DNA, 594 bp, synthetic construct
  source 1..594: mol_type = other DNA
atggattccctcttgatgaagcagaaaaaatttctttacaacttcaaaaatttgcgctgggccaaaggaaggcacgaaacctacctttgctacgttgtcaaacggcggaactctgcgacctcatgttcacttgacttcggatatttgaggaataagagcgggtgccatgtggaagttctgtttctccggtatatatcgacctgggatcttgatccacggcattgttatagaataacctggtttacgagctggtcaccgtgttatgattgtgctaggcacgttgccgactttttgagtgcgtacccgaacctgacgctgaggatctttgctgctcgcctttacttctgcgaagagcgcaatgcggagcccgaaggcctgagacggcttcacagagccggtgctcaaatagcgataatgacctttaaggactatttctactgttggaatacttttgtcgaaaaccgcaacaagacattcaaggcttgggaaggcttgcatgagaactctgtcagattggctcgcaaattgagacgcattcttcttcctctctatgaagtggacgatcttcgggatgcctttaggattctcggactc

SEQ ID NO: 94 - DNA, 690 bp, synthetic construct
  source 1..690: mol_type = other DNA
atggctgagaagcaggaacaaccaccggtcgctcaaaatggggaaaaggcggagaaggccgaaaccgccgagaaagcagagaacgcggaagaaagtgccgaagccgagaagcccaaggaattggaagagctgcctccattcgaaatcgtgacaggggaacgcctgcctgcttgttttttcaacttccaattcaaaaatgttgaatacagcagtgggagaaataagacattcctttgttacataattgagattcaggacaaggaatcgaagataatcagaggctacctggaagatgaacatgctgccgctcatgccgaggatgccttttttaatactatcttgccaaaatgcgaatcagggttgcggtacaatgtcacgtggtatgtctcgtgtagcccgtgtgtcggatgcgccgacaggataactaaggctttgcaaaagaacaaaaatctgcatctctccatcagtgttggacggctgtttatgtgggaggagccggatattcaggctgctctcaagaagatgaaaacggctggttgtaaactcagaataatgaaaccgcaagatttcgaatacgtctggcaaaacttcgttgagcaagaagaagaagaggcaaaggctttcacgccgtgggaggatatccaagagaactttcaattctacgaggaaaaattggccgagctgctgcat

SEQ ID NO: 95 - DNA, 606 bp, synthetic construct
  source 1..606: mol_type = other DNA
atgttcttgcccccctgtagcctgctgatgaaacagaaggagtttctttatcactttaagaatcttcggtgggcgaaaggtagacacgaaacttacctttgttacgttgtcaagcagcggaactccgcaacatcgtgcagtcttgacttcgggtttctgaggaataagtcggggtgccatgtggaggttttgttccttagatacataagtacatgggatctcgatcccagaaactgttaccgcatcacatggttcacctcatggtccccgtgctacgactgcgctcgccacgttgccgacttcctttgtgcttaccccaatctgacgctcaggatttttgccgcaagactctacttttgcgaggaacgcaatgctgagccagaaggacttcggagactccatcgcgcgggggttcaaatcgccatcatgacctttaaagactatttctattgttggaacactttcgtcgagaacagaaaaaaaacttttaaggcgtgggagggactgcatgagaacagcgtgagattggcccgcaaacttagaaggatactccttcctctgtacgaagtcgatgacttgagagatgcctttaggatactgggactg

SEQ ID NO: 96 - DNA, 525 bp, synthetic construct
  source 1..525: mol_type = other DNA
atgcccagtgctttcttcaaattccatttcaagaacgttgaatacagtagtgggcgccataaaacgttcttgtgttattcagttgagaaacagggtaaggaatgcgaaaggagcgagggatatgtcgaagatgagcacgcagggacacacgcagaaaaagccttttttacggatatattcacggaatgtgatggttcacttcgctactcgattatttggtatgtgtcttcatcgccttgcaccgtgtgtgcagccaagatcgcggaatatctctctaaatgcccgaatgttgagcttacgatatttatggcgagactgtttatgtgggaagaggcagagatccaccagggcctccggcaacttcaggccgctggatgcagattgaggattatgaagggcccggaatacaactactgttggaatacttacgtcgataacgagggggaagcattcacgccctgggaggaccttcaggattcgtgcactttttacgaagaaaaactccatgacatactccagcatgca

SEQ ID NO: 97 - DNA, 585 bp, synthetic construct
  source 1..585: mol_type = other DNA
atggcggccggacctgcaccagaagcacggcctttgatggatgaacaaaccttccttgacaactttaataaccttgtcaacccacatgagacatatctctgtcactacgtggaacggcaggagggagacttgtggattcctgtcgttgagttcaaggattttttgcggaatcaaggtgccgacgttgtgcttcatagatggcaacgcaggcacgccgagattatatttctggaaagaatggcatcctggcggctcgacaaggagttgagatacagggtgacatgttacatttcatggagcccttgtgaggactgtgcctataagttggcagccttcttgagggagaacacacacgtcgatcttcggattttcgcagccagaatatatgattacttgccaggatatgaagcaggcttgcgcgcgttgagggccgcaggcgcggaagtggctatgatgccgacggatgagttcgaacggtgttgggaaagtttcgtcgaccatcagggacgcccattcccaccatgggatgaccttgacgttaaaagcgaagaactttctcggagattggaaggaatacttcagaac

SEQ ID NO: 98 - DNA, 639 bp, synthetic construct
  source 1..639: mol_type = other DNA
atgattaataaattcgactctgttctcttggcacagaaaaagtttatatatcactacaagaatatgagatgggctaaggggagacacgaaacgtacctgtgcttcgtcgttaaaaggcgggggggtccgaacagtctttcatttgatttcggccacttgaggaaccgctcgggctgtcatgtggaactgctcttccttcggcttttggaggcaggagcactgtgtcccggcctttggggctatggtgccccagatagcgtcggcctttgctattcggttacttggttttgtagttggtctccctgttcggactgctcttacaggctggcgcaattcctctcgcaaacacctaatcttagactcaggatatatgtcagcagactctatttttgcgaccctgaagactcctccgctagggagggtctccggatgctgcaacgggccggggtccagataacagtgatgaattatgaggattatttttactgttggcagacgttcgtcgcttgtaggcaacgggtctttaaagcttgggacggtctgcatcaaaactcagtccaactggcccggaagttgaatgacattctgcagccgggggaagcggaggattggggcgacgccttcgagctgttgggcttg

SEQ ID NO: 99 - DNA, 885 bp, synthetic construct
  source 1..885: mol_type = other DNA
atggcggacaaaaagggggctgccgccagttcaaaattggttctcagaaaaaaagaaaggactacaaaaacggcggtcactgtcgaggtcaagaaagaagttaagaaggaagttaaaaccgaaatgaagaccgagatgaaaagggaggtgaaaagagaggtcaaatctagtctggtgaagaaagaggaaaaggtccttgcggtgggagaaaaggtggaagggaatggggaggtcccaatggaagaaggggcgacggctaatgaggatgtcgccaacggagaagctgctacggctaatgcaaatggtgctaacggcgaatatgaacctattgagcttccgccatgggaaataattgaaggtgatagaatcgacccctttcaattcaaattccaatttaaaaatgtggaatacagcagcggtcggaacaaaacattcctttgctacctcgttgataagggaaaagccgatgacgggctgatgcgcggatatcttgaagatgaacatagcggagctcatgccgagcaggcgttcttcttgcagacactgccggactacgatcccgcagtcaaatacactataacttggtatatgtcctcatcaccttgtgcagtctgcgcggccaaaattgctgaggcgcttcaagcacggaagtcaatcaagatgactctcttttctgcacggctcttcgagtgggaggaccaggatatacaagctggcttgaacgccctttcgcaggcaggctgtaagttgaggatgatgaaacctatggatttcacctacgtctgggacacttttgttgaaaacgacaacttgacgtttacaccttgggaggactgccaggataattatgagtactatcatgaaaagttggccgatattatgcag

SEQ ID NO: 100 - DNA, 603 bp, synthetic construct
  source 1..603: mol_type = other DNA
atgacaatggactccatgctcttgaaaaggaacaaattcatctaccactacaagaacctcaggtgggcgcggggaaggcatgagacgtatctctgttatattgttaaacgccgctattcatcagtgtcttgtgctttggactttggctatctcaggaaccggaacggatgccatgcagaaatgcttttcttgcgctacttgtcgatctgggtgggccatgatcctcaccgcaactacagagtgacatggttttcgtcgtggtccccttgctatgattgcgccaagagaacgctggaattcttgaaagggcatcctaatttttcattgagaattttttctgcccggttgtatttttgcgaggaaagaaacgccgaacctgaagggctgagaaaattgcaaaaagcaggagtgcggttgtcggttatgagctataaagactacttctactgctggaataccttcgtggagacacgcgaatccggctttgaggcatgggatggtcttcacgagaatagtgttaggcttgccaggaagctgaggcggattctccagcctccttatgacatggaagacttgcgggaagttttcgttctgcttggcctg

SEQ ID NO: 101 - DNA, 705 bp, synthetic construct
  source 1..705: mol_type = other DNA
atggctcaaaacaaatcacagagctcaaaggacccgatcaataacgtcgaagaagctgagaaggcggaaaaggcggagaacggcgagggtaattctaagaaaaaagaacaggaagagctgcccccattcgaaatcgtcgaaggctcacgcataccggcatccagctttatgtttcaatttaagaacgtggaatactcgagtgggagaaacaagactatactgtgctacactgtcgagcggcctgagggacaaatattccatggctatttggaggatgagcatgtgagtgcccatgccgaggatgcgttcttcacgtccgtcctgccccagttcctgacgtccggtagtgtgacagttacttgctatgtttccagttcgccttgcgtgaactgcgccgcgtcgatagcgcaatgtctcagaagatacaagacacttcgcatccaattggcggtcgcaagactcttccagtgggaggagccggagacaaggaggagtttgaaaggactgaggtccgctggctgcccggtgaggatgatgagaggagcagactatatatacgtctggaagaattttgtcgagccggacattacgttggatgaagaggggaaagaggtggttaaagatgagcaccaagactttattccatgggaggatttggaggagaatagcaggtattatgaggaaaaactgagcgagatacttcgg

SEQ ID NO: 102 - DNA, 603 bp, synthetic construct
  source 1..603: mol_type = other DNA
atgactttggacagccttttgctgaaaaggaacaagttcttgtatcattacaaaaaccttagatgggctaggggtaggcatgagacatatctttgttacatagtgaaaagaaggtatagttctgtctcgtgtgccttggacttcggttatctgaggaatagaaacggctgccacgcggaaatgctgttcctccggtacttgagcgtttgggttggacatgacccacaccgggcataccgcgttacttggttttctagctggtcaccttgctatgactgcgccaagcgcacacttgagtttttgaagggccaccctaatttttctcttagaattttctccgcgagattgtatttctgtgaggagaggaatgctgagccagaaggactccgcaagttgcaaaaagccggtgtccggctcgctgtcatgagttataaagactacttttactgctggaatacctttgtggaatctcgggaaaggcgcttcgaggcttgggatggtcttcacgagaactccgtcagactcgcacggaaactcagacgcatacttcagcctccgtatgatatggaggacctccgggatgtcttcgagctgttgggtctg

SEQ ID NO: 103 - DNA, 555 bp, synthetic construct
  source 1..555: mol_type = other DNA
atggatgaatacactttcacggagaactttaataatcaaggaagaccgtctaaaacgtacctgtgttacgaggtcgagcggctggatggagatgcaaccattccattggacgaatataaaggcttcgtcagaaacaaaggtttggaccaaccggagaagccgtgccacgctgagttgtattttctcgggaaaatacggagttggaacctcgataggaatcagcactacaggctcacctgtttcataagctggtcgccttgttatgactgtgcccagaaactcactacttttttgaaggaaaatagacatatcagcttgcacatactcgcatccaggatctacaccaataatcatttcgggtgtcaccaatctggtctttgcgaactccaggctgcgggcgcgaggattaccatcatgacgttcgaggatttcaaacattgctgggaaacctttgtggatcataaggggaagccattccaaccatgggaaggcctgaatgtcaaatcgcaagcattgtgtgcggagcttcaggcgattctcaagacgcaacagaac

SEQ ID NO: 104 - DNA, 582 bp, synthetic construct
  source 1..582: mol_type = other DNA
atggaggctagtacagccccacgggctagatgtttgatggacgaacatactttcactcataacttctccaaccataagaggactcataagacatatctgtgttacgaggtcgaaattctccacggtgacagcggcatccccccagacgaggacaagggttttctctgtaacaagggtactgatcagcctggacttccgtgtcacgtggagctgtatcttcttggacggatcagatcctggaaactcgatagaaagctccactgtaggcttacctgcttcatttcttggaccccttgtggcacgtgtgccagagaactcgctgagtttcttaaggagaactcccacgtttcgttgtgcattttcgctagtcggatctacagtctcaacgattacgaagccggtctgcgcactttgcaggaggcgggtgcacaaatcgcaataatgacctttaaagagttcaggcactgttgggagacgtttgtggatcatcagggcagaccattccaaccgtgggacgaactggacatcaatagccaagggctcagtaaagaattgcaggcgatacttcagacacaacagaat

SEQ ID NO: 105 - DNA, 1014 bp, synthetic construct
  source 1..1014: mol_type = other DNA
atgatcccgagtttgcaccaatattattccagaacatgcctccatgctgagcaatgcttcctttattggtttcatgaacacatgctggacccggctgaagattaccaagtcacatggtacatatcatggagcccgtgcagcacctgtgcagatgagatggcgagttttcttgccacacaccgcaacgtgtctttgacaatattcagtgcccgcctttactacttctggaacagcgcataccaaaacggacttcggaaactttaccaggagggtgcccaggtgaaggttatgacattccaagaattcgaggattgttgggaaaattttgtctacaataagggtgagagttttatgccctggaaaggactctcaaggaacaacaggtttttgaaaaatgaactcaaaaaaatcctcggttctccgatgtctctcctgaaggaagacatattcctctatcagttcaacaaccaacaacaggttcaaaagccgtacttccgccggaggacctatttgtgttatcagttggaacagcccaacggcagccgccctcaatggccggcgaaaggttgtcttcagaataagaaaggacatcacgccgagataaggttcatcaagcggatacactctatgggactggagcaggaccaggattaccagataacttgctacattacctggtcgccttgtctggcctgtgcttgtgcgttggccgaactgaagaaccacttccccagacttacccttcggatctttgcgtcgaggctgtatttccattggataagaaagtttcaaatgggtctgcagcatctttacaagagtggggtccttgttgctgtcatgagcctccccgagtttactgactgctgggaaaaatttgtcaatcaccgccaagtgttttttaccccttgggacaagctggaagagcattcgcggtctatacagaggcggctgaggaggatcttgcaaagttgggatgtcgatgacctcacggacgattttcggaatcttcggttg

SEQ ID NO: 106 - DNA, 570 bp, synthetic construct
  source 1..570: mol_type = other DNA
atgaacccccaaataaggaacccgatgaaagctatgtatccagggactttttactttcagtttaaaaacctcagagaagccaacaaccgcaatgaaacctggctgtgctttaccgtggagggtattaggcaccatctcaccgtttcttggaagaccggggtgttcagaaatcaagttgacctcgagactcactgccatgcggagaaatgcttcctgagttggttctgcaagaacatcctttcgccaaacacggattatcaggttacatggtacaccagctggagcccctgtccagagtgtgctagagaagtggcggagttcttggcacgccataataacgtgatgttgaccatctacacagctcggctttattattcgcagtgtccgaactatcagcaagggctccgctcgcttagcgaaaagggggttagcgttgaaataatggattacgaagattttaaatactgttggcagaattttgtgtatgatgacggagagcccttcaagccttggaagggtcttaaaacatcgttccggttcttgaaacggtgtcttagagagatcctccag

SEQ ID NO: 107 - DNA, 654 bp, synthetic construct
  source 1..654: mol_type = other DNA
atgtcttgcggatctagcagggatcagacggtcctttttccgtttgcgcttacgagcccaagagcaacaccccttcctgccacccaccgcgctccggtgtgttggaataggaataggaaagaagggagtactaagggccgcaaacagtgctggactgtccatggtgaacgccggggtcatgcccaggagaaggccgtgaggtggggcgacaccggtaggccttggatacattggattcggaaggaccattaccatgcagaagtctacttccttgaaaaaatttttaagatgcggcggagtaaaaattttgtgaactgctcaataacatggtacctttcctggtcgccctgtgtttcttgctgttgcgagatccttaacttccttgagaagcattctaatgttaatatagatatttacgtcgcccaactctacaaaatccagaattccgaagtttgcgaggggctcaaaaagcttgtgtcactcaaaaaagtgacgatagcagtcatggaaatcaaggattatacctattgctggaagacattcattcaaggcgacgcggacgacgactcttggaccgttggattccagtcggcaattaccaagaaccgccttaaacttaaggacgtgttcgaagatctcccgtttattcagtcg

SEQ ID NO: 108 - DNA, 723 bp, synthetic construct
  source 1..723: mol_type = other DNA
atggaggggagcgcggaaccaaaaccgtcggacctgctgagcccggaagacttcactttccattacgaaaacttgaaatatgctcacgagagacggaagacctatctgtgttatcatttggaacgccttgacggagacagaagagttatgttggacagcgctgtgattcaaaaccagggatcgagacacgttgaagaatgcttcctttttagtgagaatgttctggaaaaattgaaagcaccgggcagttatagcgtcacgtggtacttgagttggtcaccatgcgcacggtgtgcccaagcagttatcagatttctgcaacagcacccgaatgttcggctgaggatattggtcgctaggctctattttcatgaaacaagagcgaatagggccgcacttcgggcactgagacgcagggaagcaccaattgagatcatgagtcccgaagggcagctcccgtggggacacggaggggccggccaggccgctagaccgagactctgtcttttgttggaaacccttaggggtccacccagaggagggttgcctgccctgggtggttatggtaggaaacggggtccgccaggacggggagcagagagacacctggcagttcaggtgaagagggcaagaaaaattgggaatcaagaaatggatctcgagcctgataagaagtccacttggatgtcgatccctccccggtggctgcttgaagatactccc

SEQ ID NO: 109 - DNA, 582 bp, synthetic construct
  source 1..582: mol_type = other DNA
atggaggcgtccactgcgccctggacacggtgtctgttggatgaaaacacgttcaatgaaaacttcatgaaccaaatcaacccaagaaaaacttatctttgctacaaggtcgagatacttgatggcgacactagggtgctgcttgacgagaagaaggggtttgtccagaacaaaggagcaaacgaacctggaaaacccagacatgccgaatcgtacttcttggacagaatcaggtcctggaatcttgaccggggacttcactatcgccttacctgttttatctcatggacgccatgccatagctgcgcgcaggaacttgcaacgtttttgggcgaaaatagtcacctgagcctccatatctttgcctcgcggatttactcgctgccgggctacgaagccgggctgaggaagttgcaggccgctggagcccagattgcgataatgactagcaaagaatttgagcactgctgggaaaatttcgttgaccatcaagataggccattccagccctgggacggtttggaggttgtttctcagcacctctgcaatagtctccaaactatactgcagatgcaggagaat

SEQ ID NO: 110 - DNA, 624 bp, synthetic construct
  source 1..624: mol_type = other DNA
atggcgcgcaagaaattttctcgcaaggagggttatatacctaagccgtggcctagaaacccaatggagaagctgtatgctgattattttaatttccactttacgaactggcctactcctaagggtaggaacggttgctacatctgttatgaggttgagcgccggcttcataattcacccatcagtctggttaggggtgtctttgaaaacgagttctatccaaaagaacccgtccacgccgagctttgtttccttaattggtttaaggaggagtttctcgagaagacactggaacctggtggtatttactgtgtgacgtggtatacaagctggtcgccatgcgtggaatgcgcgaggcaagtggcggaatttttgaacacacataaatatgtgaggttgaggatcgtttcatgcagattgtatcattcaaataaacctgagcatcaacaagggctcaggaatttggcgggtgcggaagctacactggccgttatgagccctgaggacttccattattgctgggataactttgttgacaaccgcgaaagtgatttcacttactggaggggaatcaggcggaactactacggtttgcatgagagactgcgggacatccttagtggt

SEQ ID NO: 111 - DNA, 642 bp, synthetic construct
  source 1..642: mol_type = other DNA
atgagtacacaaataagaacagcatgtgggcatccaccgggtggccaccagcagagaaagcatgcgtttctcaagttggttcagtcccttatttccccgagggatttcttgttccaatacgaaaacctgcaatttgcggacggacggaatatgacttatttgtgctactgcgtggaggagaagaacggagacactccaatgcttttgcacattgatgtcttgcagaacgaggttgctggatctcacgtcgagatccttttcctgagctttatgaggcgctttctcgcttcaatcgatcagtccttgcaagaggccagagagtcccaggtcacatggtatatatcatggtcgccgtgcgccgactgcgcgaacacggtggctacctttttgagagaacggaataatgttcacctcaggctgttcgccagtcggctgtattatagggacgtgcccgagtaccaacaaggcctgagggcactcaaggaggcgggggcacaggtggagatcatgtcttcattggaattttctgactgttgggatcacttcgtggatcatcaaggtgaatgtttcgagccgtgggaggaaatggatgagaatgttgaaattttctcccaaaagctgcaggatatcttggaaacacgc

SEQ ID NO: 112 - DNA, 687 bp, synthetic construct
  source 1..687: mol_type = other DNA
atggatcacagaatatttatatatcacttcaagaacgaccagagcgtcagagggagacatatgacttatctctgttacgaagttcaactcttcgacgttcggtttcgcgttcctctcggcatgattagaggtgtgctccataaccagcatcaccccgatcctcaacggagatgtcatgcggagctctgcttcctcatcaggatgagctcccttcgcctgtatccagggcagtggtgccaagttacttggtttcttagctggagcccctgcccgagatgtgcatccaaagtgtcagattttcttagaagtaaccgccacgtcagcctgaggatttttgcagcaaggctgcatacataccatgccggctatgaagaaggtctgcggtcactcagagatgctggggcgcagttgtctattatgacggcaaaagagtttgagtactgttggagagtcttcgtggataaccattcgtggccattcagaccgtggaacatgttggagaagtcgtctcaggccatatccgagaggctccaatccattttgcgggagaaaattagtttcttccatcaaccgacctgctaccaacctagctttggtgttttctcgccgggtaggtttccatgcccaccgcagaccgcaagtagctggtttcccgctcctcctcgcgggccccagcaccatccattt

SEQ ID NO: 113 - DNA, 585 bp, synthetic construct
  source 1..585: mol_type = other DNA
atggagccaactccaggaccctattcgcaatgtgttgatacttgtccctcatcacctctgtcactcgccttggcgggacaatcggacttgataagggaagattactccgaaaagagtaaggctaagctgggcgacgaggacagtcggaacattgatttcaacttccatttcgaaaacttgcaaaaggcctttgagcgcaacatgacttacctgtgctataggcttgagagactcgattcctctggtccggtgctgttggatgcggctgtgctcgaaaatgtgccaggaacacacgttgaaatgctgttcctcaatagccgcaacattcaggagttgaaacaggacaaaaaatatagggtgacatggtacatatcttggtcgccttgtgccaaatgcgcccaaaaagtgagtaaattcctcgaagcacattctaatgtgagcctgcatatttttgcgagtcatctttattactgggataagccagaaaccaggcagggtcttagagatctgcaacaggcggaggcgcaaattcaagttatgagcagcttgggggagctggcgcactggatggcagcgggccgcatt

SEQ ID NO: 114 - DNA, 879 bp, synthetic construct
  source 1..879: mol_type = other DNA
atgttcggaggaggagggtcttttattacgcacttctgtattgagatgataggcggcggaggaggccgcgtcttgctgaagcaacaaagccgggcactgagagaaaggacgggagctccaccttcacctcaagcgccaccaccatccccttgtcccccctcgttgccaaggttgcccacctggaggcaacaggagatgctcaagggacggggctgctgggaagcaggtattccgggccatctcataggtgagacaggtggcaatccggcgttctcgttgtttagcccagctccaagaaacccgatgagatggctgtcactggactatttttatttccatttcaccaactggccaaatccgaaggggagaaacggatgctacttgtgctatcaggttaagagaacacaagaagggctcagttgggatgttggcacgggtgtgttcgagaatgaattctatcctaagaaaccagttcataccgagatttgcttccttaactggtttaaaacacagcaggcgtttttggcgcaaaatctttcaagggaagagaattaccatgtgacgtggtttatgagctggtccccctgttttcaatgtgcgcgccaggtcgttgcgttcttgagagatcataagcatgtgcagcttagcatcttcgccgctagactctattacagcaggagtccacagtaccaacaaggcctcaggtccctgcagggcgcaggcgcgcaagttgctattatgaccccagcggatttcgctttttgttgggacatttttgtggacgatccccttaggccgttccgctattggaaaggaatctacattaattcttgctctttgagcaaaagtcttgaggatatcctgcggaatcgggaaaat

SEQ ID NO: 115 - DNA, 690 bp, synthetic construct
  source 1..690: mol_type = other DNA
atggatccgcaggcacccactcagcgggggggactcggtcaagcataccagggaggtgactatgtccaggcaccaggaaatggaaatactcagcatctgttgtcagaagacgttttcaaaaaacaattcgggaaccaaagaagggtcaccaagccttactacagaagaaaaacctatgtgtgttaccaacttaagctccttcgcgggcccactatcgccaaggggtatttcagaaataaaaaaaagcgccacgcggaaatccgctttatagacaaaataaactcacttggtctggaccaggaccaatcctacgagatcacgtgctatgttacatggtcgccatgtgctacatgcgcgtgtaaactgatcaaatttacccgcaaatttcccaacctctcgcttaggatattcgtttcccgcctctactaccattggttcaggcaaaaccagcaggggttgcggcagctttgggcgtcaagcatccctgtcgttgttatgggctatcaagaattcgctgactgctgggaaaacttcgcggataatcggggaaatccttttcagtcctgggaaaagttgaccgaatactctaaagggatcaagcgccggttgcagaaaatactcgaaccgcttaatctgaacggtcttgaggacgcaatgggcaacttgaaactcggttcagtggacctcggc

SEQ ID NO: 116 - DNA, 690 bp, synthetic construct
  source 1..690: mol_type = other DNA
atggaggcatctgctgcacccatggcaagaagactgatggacgaggatacttttacagagaacttcaaaaacgtgaactggcctcgcaagacctacctttgctatgaggtcgagctcccagatggcgattccagagttccgcctggctgggacaagggttttttgagaaacaaaccaattcacatgcccgggcctccccgggacgccgagatgaggtttttggacctcatttcgtcatggaagctcgaccagaaattgcgctatagggtgacttgcttcataagctggtcgccttgcgccgactgcgctcaacggctcgcagaatttcttcgggagaattcccacgttagtcttaggatatttgcctcgcgcattttcacaaaaggagactacaaagcggggctccgcacgcttcaagcagcagaggcacaaattgcgatcatggcttctgaagaattcgaacactgctgggaaacgtttgttgaccaccaaggaagaccgttccaaccgtgggacgaacttgatccaaagtcccggtacctctctgagcagctgctccatattctccagcccaccgctccgcccagtttgccgcccctttactttccgccaagcctgcccccattgtacttcccgccttcccatccagttccgggaccttacttcaggtcgctgcgcgatagttcgggt

SEQ ID NO: 117 - DNA, 582 bp, synthetic construct
  source 1..582: mol_type = other DNA
atggaggcaaatccctccgctgggacgagttgcttgctggacgagaatacttttacagagaattttatgaatcagatgaatccccggaaaacctacctttgctacaaagtggaaatattggatggtgatgcaagaataccactcgacgaaaaaaaagggttcgtcaggaacaagggggcgaacgaacctggtgaaccatgccatgctgagcactactttctggatcgcatctgctcctggaaccttgaccgcgaacttcattaccgcctcacgtgcttcatttcatggacaccctgcgacacttgtgctcagaagttggctgactttcttgggaagaatagtcacgtttctctgcacatctttgcaagcaggatatactcacggttggggtatgtggcgggacttcgcaccctccaagccgccggagcccaaatagcgattatgaccagtaatgagtttgaacattgttgggagaacttcgtggaccaccaaggtaagcctttccagccgtgggacgggttgaacgttgtttcacagcgcctttgcaacaatcttcaagcgattcttcaaacgcaagaaaat

SEQ ID NO: 118 - DNA, 570 bp, synthetic construct
  source 1..570: mol_type = other DNA
atgaacccccaaattaggaaccctatggaacgcatgtataggccaactttttattacaactttgagaatgaacccatactttgtgggcggtcctacacatggctctgttatgaggtcaaaataagaaaagatccatcaaaactgccctgggatactggcgtgtttaggggacagatgtattttcaaccggaataccatgctgaaatgtgtttcctgagttggttctgtggaaatcagctgcccgcgtataagagattccagatcacttggtttgtcagctggactccctgtcccgactgtgtggcaaaggtggccgagtttcttgcagagcatcccaatgttactctgactataagtgccgcgaggttgtattattattgggagaaggactggcaacgggcgctgtgtcgcctttcccaagccggagcgcgggttaagataatggattatgaggagttcgagtattgttgggaaaacttcgtgtataacgagggagagccgttcatgccgtggtacaaattcgacgctaattacgcttttttgcatcacactctgaaagaaattttgcgg

SEQ ID NO: 119 - DNA, 654 bp, synthetic construct
  source 1..654: mol_type = other DNA
atgtggggagctgagggcaaatctgtcagaggaggagaagagaggtgggcagttcctgaaggagcaccatgtctttgggcatctccagcctttgacagatcctgcctgctcgatgaaaacacattcactgagaattttatgaaccaaatgtccccacgcaaaacatacctctgttacaaggtggagatacttgacggagatgctagaataccactcgacgaaaagaagggcttcgtccggaataaaggtgctaatgagcccggcggaccgcgccatgctgaaagttactttctcgaccgcatcagatcttggaatttggaccgggacctccattaccgcctgacctgttttatttcatggacgccttgtgatacttgtgcgcaagaactggctgattttctcggaaaaaactcccatgtgtccctgcatattttcgcatctaggatttactctcgctttggatatgaggacggattgagaacacttcaggcagctggcgctcaaattgccattatggcttctaatgagtttgagcattgctgggagaactttgttgaccaccagggaaagccctttcagccctgggacggcttggaagtggtgtctcaacgcctttgcaataaccttcaggcaatattgcaaacgcaggagaac

SEQ ID NO: 120 - DNA, 711 bp, synthetic construct
  source 1..711: mol_type = other DNA
atgtcatttgaggattatgagtattgctgggacaattttgttgaccatagaggcatgagattccagtcgagaaatctgttgagagataacgatctcctcgctacggaacttgaaaacatcttgaggacaacgatgaatcccctgcaaaaagaaatttttttttaccagtttggcaatcaaccgagggcgccgaaaccgtatcacaggcggacaacatatctctgctatcaactcaagccccatgaggggcctattatagcccgggtgtgtttgcagaataaaaagaagcgccacgcagagatcagattcatcgataacataaaagctcttcgccttgatacgagtcaacgctttgaaataacctgctatgttacgtggtcgccctgccctacgtgtgccaaagaattggtggccttcgtgcgcgaccaccggcacatctcgctgagactgttcgcttcacgcctctattttcattggttgagagagaataaaaaaggactgaaaagactccacgcatctaggattcccgtggcggtcatgtccagcttggaattcgaggattgctggaaagaatttgtcgacaaccaggggaagccttttcaaccctggaataagctggagcagtacagcgaatcaatcacacggcggctccgcaagatacttgagccgcagaacgatctggaaaacgaattccgcaatcttcaactgcaa

SEQ ID NO: 121 - DNA, 465 bp, synthetic construct
  source 1..465: mol_type = other DNA
atggccacatctcccacacttcctccagccctgcaaccgagagggagtccctcactcctcaggctctctcaggtgtatccacaggcgccttgtcacgctgaggcttgttttctgagttggttcagggatcaaaacttgccgcctgaagagcactatcgcgtcacatggtttctgtcctggtccccatgccccacatgcgcggaggacgtcgtggaatttttggaagagtacaggaacgtcactcttagcatctacactgcaaggctttactacttttgggatccagatttccaagatggtcttcacaagctgtggtgtgcgggggtccaccttgacatcatgtccttcgaggactatgagtattgttgggaaacattcgtggatcacaaaggcatgagattccaatcgtgggatttgcttcgggacaatgacttgctcgccgctgaattggataatatcctcggg

SEQ ID NO: 122 - DNA, 552 bp, synthetic construct
  source 1..552: mol_type = other DNA
atggcgaaggggatattcctcccagacttcactaacgacccttctcaattcagaggcttgaaacgcacctacctttgttacgaggtcgaactcttggacggtaactcttgggtccctctcgacaacgggagggactttttgtctaatcagccacggcggcatgcagaactgtgctttctcgatcgggtttcgtcctggcatctggaccccaccaagaggtacaaatttacttggtttcttagttggtcgccgtgcaggaactgtgcacaagaggtggctgctttccttggcggaaacagccatgtctccttgtccatcttcgctgcgaggatatatgactgctaccccggttatgaagaaggtctgaggtcattgcaaggagctggagcccacgtggctattatgacatcgactgagttcgagcggtgctggcggacgttcgtcgacaacggtggatgtctgtttgtcagttggaacgggctgaatgaaagctcgcaaatgcttaaacggcgcttggagtcaattctccagggtcaactcagaaggcaccca

SEQ ID NO: 123 - DNA, 633 bp, synthetic construct
  source 1..633: mol_type = other DNA
atggcgctcctgacagcagaaacttttagtctgcaattcaacaacaaaaggcgggtcaacaaaccacactacccccgcaaagcgcttctttgctaccagcttacgcctcaaaatgggagtacacccacgagggggcacctcaaaaataaaaaaaaacatcacgctgagatccattttatcaataagatcaagagtatggggcttgacgaaacccaatgctatcaagttacgtgttatcttacctggtcgccctgtccatcttgtgcaagggagttggtggacttcatcaaagcgcatcgccatctgaatctccggatcttcgcctcaagactttattaccattggcggccccactatcaggaaggacttttgcttctctgtggctcacaagtgccagtggaggttatggggctcccagagttcagcgactgctgggaaaatttcgtcgaccacaaggaaccgccatcctttaatccttctgaaaagctggaagaactggacaaaaattcccaggctataaagaggcggctggagcgcatcaagcagtcgcgcagcgttgacgtgctcgaaaacggacttagatcgttgcaactgggaccagtcaccccaagctccagtattagaaattcgcgg

SEQ ID NO: 124 - DNA, 570 bp, synthetic construct
  source 1..570: mol_type = other DNA
atgaacccgcagataagaaatccgatgaaagcgatggacccaggcaccttctactttcaatttaaaaatctgtgggaggctaataacagaaatgaaacctggttgtgtttcacagtccaagtcattaaacaccactcaacagtgagttgggaaaggggtgtcttccaaaatcaagttgatctggaaactcattgtcatgccgagaggtgctttctctcgtggttttgtgaggacattcttagtcctaataccgattacagagtcacttggtacacttcttggtcgccgtgtccggaatgcgctagagaagttgctaaatttttggcacgccacaacaacgtcatgcttactatatatacggcacggctgtactactcgcaatatcctaattatcaacaaggcttgcgcagactctctgaagaaggtgtttcggttgagattatggactatgaggacttcaaatactgctgggaaaattttgtgtatgacgacggcaaaccgttcaagccctggaaagggttgaaaactaattttcgctttctcaaaagaagacttcaagaaatacttgaa

SEQ ID NO: 125 - DNA, 729 bp, synthetic construct
  source 1..729: mol_type = other DNA
atggaggctagtactgctccgtggaccagatgcctccttgatgaaaatacattcacagagaatttcatgaatcagataaatccgaggaaaacgtacctgtgctataaagttgagatccttgacggcgacacgcgggttcccctcgacgaaaaaaagggcttcgtccataacaaagttaccgatccggcctgcccacagcaagcgggaccgccctattgtggaactctgagagtggagggctgccaacttcataccgggtgcccgacatccagcttgacgccaggcccgtgccggtgctactgctgttcagcgtggaatcagggggcgaacgaaccgggaaaaccctgtcatgcggagtcctactttttggacttgataaggtcttggaatctggacaggggcctccattaccgcttgacatgcttcattagctggaccccatgtcacagctgtgctcagaaattggctacgttccttggtgagaattcacatttgtcgttgcatatattcgcttctcggatctactcacttcctgggtataaagctggcctgcgcaaattgcaggcggcgggtgcccaaatagcaatcatgacctctaaggagtttgaacattgttgggagaactttgtggatcatcaagataggccctttcagccttgggatggactcgaggtcgaaagtcaacacctctgtaacaacctccaagttattctccaaatgcaagaaaac

SEQ ID NO: 126 - DNA, 729 bp, synthetic construct
  source 1..729: mol_type = other DNA
atggaagcatccactgccccatggaccagttgcctccttgacgagaatacctttaccgaaaactttatgaatcggctccggcccaggaaaacttacctgtgttacaaagttgagatcttggacggagatgctagagtgccgttggatgaaaagaaaggctttgtcagaaataaagtcacggatcctgcctgcccgcagcaagcaggtcctccctattgcgggactcttcgcgtcgaagggtgccaattgcacacgggctgcccaacctcttccctcactcctggcccttgcaggtgttataggtgcagcgcctggaatcagggagccaacgaacccggtatgcctcgccatgcagaatgttatttcttggaccggataagatcgtggaacctcgatcgcggcttgcattaccggctgacttgttttatctcctggaccccatgccacagctgtgctcaagaactcgccacatttcttggcgaaaattctcatgttagtttgcatatttttgcttccaggatataccgccgccctggatatgaagcgggaatcctgacgctcagggccgctggagcgcagatcgcaataatgacatccaaagagtttcagcactgttgggagaatttcgtggaccatcaggagcggccatttaggccatgggttgggcttgaagttgagagtcagcatcagtgtaatgaattgcaggcaattctgcaaacgcaggcgaac

SEQ ID NO: 127 - DNA, 582 bp, synthetic construct
  source 1..582: mol_type = other DNA
atggaggcgagcccgtctcccggtactagctgcctccttgatgagaatacattcaccgaaaactttatgaaccaaatgtcgccaaggaaaacgtacttgtgctataaagtggaaatcttggacggcgatgccagaataccgctggacgaaaagaaaggatttgttcgcaacaaaggggctaatgaacctggggaaccctgccacgccgaacattatttcctcgataggataagatcatggaacctggatcgggaattgcattaccggcttacgtgcttcatttcctggacgccatgtgatacgtgtgcccaaagactggctgatttcctgggcaagaactctcacgtctcgttgcatatattcgcctcgcggatctattcactttccgattatgaggcgggacttaggacattgcaagcggcgggcgctcagattgcgattatgacctcgaatgaatttgaacactgctggcagaatttcgttgatcaccaaggaaaaccctttcaaccgtgggatggcctcgaggtcgtctcacagcatctctgtaataacttgcaggctattctgcagacccaagagaat

SEQ ID NO: 128 - DNA, 633 bp, synthetic construct
  source 1..633: mol_type = other DNA
atggcgctcttgaccgcagaaactttctccctgcagtttaataacaaaaggagggtcaacaagccctattatccgaggaaggcgttgctctgttatcaactgacaccgcagaacgggagcacgccgacaagaggtcatttgaaaaacaagaaaaagaaccatgcagagattcggttcattaacaagataaagtcaatggggctggacgagacgcagtgctaccaagtgacatgttatcttacgtggtccccctgcccttcgtgcgctgggaaacttgtcgattttatcaaggcgtaccaccacctgaacctgtctatattcgccagtaggctttattatcactggaggccaaactaccaagaagggttgctcctgctctgcgggagccaagtgccggtcgaggtcatgggcttgccagagtttacggactgctgggagaatttcgtcgaccatgaggagccaccttcgttcaaccccagcgagaaacttgaggagcttgataaaaatagtcgggcgataaaaaggcgcttggaacggatcaagcagtccaggtcggtggatgtgcttgaaaatggtttgcgctcgctccagttgggtccggtgaccccttcatcttctacacacaactcaaga

SEQ ID NO: 129 - DNA, 801 bp, synthetic construct
  source 1..801: mol_type = other DNA
atgtggggagctgaaggcaaaggtgtccggggcggagaagaaagatgggccgttcccgaaggggcaccaaggcttagagcgtcgcctgcctttgaccggtcgtgtctcctcgatgagaatacattcacagagaatttcatgaaccgcataaggccccgcaagacgtacctgtgctataaagttgagattctggatggtgatgcccgcgttccgctcgacgagaaaaaggggttcgtcaggaataaggttacggatccggcgtgcccgcaacaagctggcccaccctacagaggcacattgagggttgagggctgtcagctgcatacgggatgtcccacttcttcccttacacctggtccttgtcactgctaccgctgcagcgcatggaaccagggcgccaatgagccgggtatgcccaggcatgcggaatgctattttttggataggattagaagctggaatctggatcggggactgcattaccggcttacatgctttatatcctggactccttgtcattcatgtgcccaagaacttgccacatttcttggtgagaatagtcacctgagcctgcacattttcgcctccagaatctatcgccgccccgggtacgaagcagggatacttaccctccaggccgcaggggcgcaaatagcaattatgacgtcaaaggagttccaacattgctgggagaactttgtcgaccaccaggagcgcccttttcgcccttgggtggggctcgaagtggaaagccagcaccaatgtaatgagctccaggctatcttgcagactcaagctaac

SEQ ID NO: 130 - DNA, 840 bp, synthetic construct
  source 1..840: mol_type = other DNA
atggaggcaggtacggcgcccaggtcgcggtgtctcttggacgagaacaccttcaccgaaaacttcaagaatcagattaacacccgcaagacttacctttgttacgaagtggaaattgtcgacggggatgccagggtcccgctcgatgagatcaaaggttttgtccacaataaaggagccaatcaaccgggggaaccatgtcacgctgaactctactttcttgaccgcattcactcttggacgctcgacagaaggttgcattacagactcacctgcttcatttcatggaccccatgccacacctgtgcgcaggagcttgccacattcctgggtgagaatagtcacgtctcactgcacatatttgcgtctcggatgtataggcgcttcgattatgaagcagggctgagacggctccaagcagcaggcgcacagatcgctataatgaccagtaaggaattcgagcactgttgggaaacattcgtggaccaccagggacgccctttccagccttgggacggactcgaggtcgagagtcaacacctctgcaaaaagctgcaggaaattctccaagtcagagctccttcatgttgcttccccggcttgcctccttcaagtcccctcctcagctcaccatgcgccttctttttgctcagtgagccaccactcggtgatttgttgccccccggagcgcctgcaccgtcattcccttcgttcctgatgagatctctgtatttcctgacgtgttgtgttcctccgtcttttcctatatttgttactttgcccaggcctcccctcgggttctgttttatcttgcaaatgtctcactccttgctcccatca

SEQ ID NO: 131 - DNA, 594 bp, synthetic construct
  source 1..594: mol_type = other DNA
atggcagcgggtccagccccggaggctcgctcacttatggatgagcaaacctttttggacaactttaacaatctcaaatacccgcggaagacctacctttgctataaagttgaaaggcttgtgggggaaaactccatccctgtcgatgaatacaaggggtttctgagaaatgaatcatcaccccgcaggtgggagcggcgccacgcggagccgattttcctggaacggatggcctcatggaaccttgacactgagctccggtatagaataaccgtgttcatatcatggagtccttgtccagaatgcgccgatgagcttgttaaatttcttcgcgaaaacaggcacgtgaaacttcgcatctttgccgcccggatctacgactggtatcaaggatacgaggccgggcttagggctcttaaagccgctggggcggaagtcgcaatgatgacattccaggagtttgaatattgctggaaaaacttcgttgaccatcaacaagatgaagacaaacccttcccctcttgggacaatttggttgtccgctcagaagaacttagccaaaggcttgcggggatattgcagaacagggaagaa

SEQ ID NO: 132 - DNA, 795 bp, synthetic construct
  source 1..795: mol_type = other DNA
atggacacagggatctttctttgttacttctcaaatgacccacgggcaagggagcagtataggacgtacctgtgttacgaggttgaactcctcgacggaaactcatgggtgttgctctataagtccaaaggttttcttcccaaacagcttgaccctgcccagtgtcatagagttacttggttcatgagccgcagtccttgcccggattgcgctcgggaagttactgcgtttctgggaaagaacagccatgttcatcttcatatattcgccgccagcatctacgattacagtggaggttacgaagaacaacttctctcgttgtggcacgctggagctaagatagcaatcatgaccccaacagatctgatgcatcaggacatttttcagtttaacttcaacaacaattctctggtgtgtggtcaaaaacaaacgtacctttgttacgaagttgagcttctcgacgataactcctgggtcttgctggagggtggacgcggcttcctcaacaaccaactcgaccctgctcaatgttacagggtcaactgctttatgtcacggcgcccctgtttcctctgcgcttgggaagtcgcaactttcctcgggcggaactcccatgttagccttcatatattcacctcatgcacgtacgagtatttcccttccagcgagtgtaagcactattggagaacatttgtggataaccagggatgtccctttgaaccatggaaccggctcgacaaaaattctcaaaccatttggaaacgcctccagaggatacttcagaatgaatactac

SEQ ID NO: 133 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 134 - DNA, 4104 bp, synthetic construct
  source 1..4104: mol_type = other DNA
atggacaagaagtactcgatcggcctcgctattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgac

SEQ ID NO: 135 - protein, 83 aa, synthetic construct
TNLSDIIEKETGKQLVIQESILMLPEEVEEVIGNKPESDILVHTAYDESTDENVMLLTSDAPEYKPWALVIQDSNGENKIKML

SEQ ID NO: 136 - DNA, 249 bp, synthetic construct
  source 1..249: mol_type = other DNA
acaaacctcagtgacatcatagagaaggaaactggtaagcaactggttattcaagagagcattctgatgctccctgaggaagtcgaggaagttataggaaacaagcctgagagcgatatactcgtgcatacggcgtatgacgagagcacggatgaaaatgtgatgctcttgaccagcgatgcgccagaatataaaccctgggcactggttattcaggactctaacggggaaaataaaatcaagatgctc

SEQ ID NO: 137 - protein, 7 aa, synthetic construct
PKKKRKV

SEQ ID NO: 138 - DNA, 21 bp, synthetic construct
  source 1..21: mol_type = other DNA
ccgaagaagaagaggaaggtt

SEQ ID NO: 139 - protein, 16 aa, synthetic construct
KRPAATKKAGQAKKKK

SEQ ID NO: 140 - DNA, 48 bp, synthetic construct
  source 1..48: mol_type = other DNA
aagcggccagcggcgacgaagaaggcggggcaggcgaagaagaagaag

SEQ ID NO: 141 - protein, 16 aa, synthetic construct
SGSETPGTSESATPES

SEQ ID NO: 142 - DNA, 48 bp, synthetic construct
  source 1..48: mol_type = other DNA
tcaggttctgagacccctggcacaagtgagtcagcaacacccgagtcc

SEQ ID NO: 143 - protein, 7 aa, synthetic construct
GDGSGGS

SEQ ID NO: 144 - DNA, 21 bp, synthetic construct
  source 1..21: mol_type = other DNA
ggagacggatcaggcgggtca

SEQ ID NO: 145 - protein, 4 aa, synthetic construct
SGGS

SEQ ID NO: 146 - DNA, 12 bp, synthetic construct
  source 1..12: mol_type = other DNA
tcaggtggctcc

SEQ ID NO: 147 - DNA, 29 bp, Oryza sativa
  source 1..29: mol_type = genomic DNA
ctcccacccctccatctcctccacggccg

SEQ ID NO: 148 - DNA, 29 bp, Solanum lycopersicum
  source 1..29: mol_type = genomic DNA
cttcagcctcctaagcttaaactaggtga

SEQ ID NO: 149 - DNA, 29 bp, Solanum lycopersicum
  source 1..29: mol_type = genomic DNA
ttcaagatctcagcgcgatggttggggag

(19) United States
(12) Patent Application Publication
(10) Pub. No.: US 2024/0327859 A1
(43) Pub. Date: Oct. 3, 2024

(54) CYTIDINE DEAMINASES AND METHODS OF GENOME EDITING USING THE SAME

(71) Applicant: University of Maryland, College Park, College Park, MD (US)

(72) Inventors: YIPING QI, Potomac, MD (US); SIMON SRETENOVIC, College Park, MD (US); MICAH DAILEY, Durham, NC (US); YANHAO CHENG, Greenbelt, MD (US)

(21) Appl. No.: 18/573,013

(22) PCT Filed: Jul. 5, 2022
(86) PCT No.: PCT/US2022/073422

§ 371 Date: Dec. 21, 2023

Related U.S. Application Data
(60) Provisional application No. 63/218,202, filed on Jul. 2, 2021.

(51) Int. Cl. C12N 15/82; C12N 9/22; C12N 9/78; C12N 15/11

(52) U.S. Cl. CPC C12N 15/8262; C12N 9/22; C12N 9/78; C12N 15/11; C12N 15/8213; C12Y 305/04005

(57) ABSTRACT

The present disclosure relates to compositions and methods that are useful for the targeted editing of nucleic acids, including editing a single site within the genome of a cell or subject, e.g., within a plant genome. The disclosure provides base editing fusion polypeptides of a DNA binding domain, e.g., Cas9, and a cytidine deaminase domain. The base editors perform equally well or outperform existing technologies in C-to-T base editing efficiency while maintaining low frequency of introducing C-to-A and C-to-G byproducts.

### CROSS-REFERENCE TO RELATED APPLICATION

This application claims priority to provisional application U.S. Ser. No. 63/218,202, filed Jul. 2, 2021, which is hereby incorporated herein by reference in its entirety.

### GOVERNMENT SUPPORT

This invention was made with government support under 20183352228789 awarded by the United States Department of Agriculture (USDA). The government has certain rights in the invention.

### SEQUENCE LISTING XML

The instant application contains a sequence listing, which has been submitted in XML file format via electronic submission and is hereby incorporated by reference in its entirety. Said XML file, created on Jul. 1, 2022, is named P13962WO00.xml and is 191,543 bytes in size.

### TECHNICAL FIELD

The present disclosure relates to compositions and methods for targeting and editing nucleic acids, in particular cytosine base editing.

### BACKGROUND

Genome editing introduces desired changes in the genomic DNA sequence that is possible due to a collection of technologies amongst which clustered regularly interspaced short palindromic repeats (CRISPR) has become the technology of choice due to its simplicity of targeting DNA. CRISPR, originally an adaptive bacterial and archaeal defense system, consists of CRISPR-associated (Cas) endonuclease 9 and synthetic single-guide RNA (sgRNA/gRNA) that is constructed of crispr RNA (crRNA) and trans-activating crRNA (tracrRNA). sgRNA directs sgRNA/Cas9 endonuclease complex to the target site in the genomic DNA where the double strand break (DSB) is introduced. Error prone non-homologous end joining (NHEJ) and microhomology-mediated end joining (MMEJ) DNA repair pathways can be utilized for knocking out targeted genes through introduction of insertions and deletions (indels), substitutions or other DNA rearrangements at the DSB site. Error free homologous DNA recombination (HDR) offers insertion of template DNA through homologous recombination facilitating more precise DNA modifications. However, HDR is not efficient in plant cells, which motivates the exploration of alternative precision genome editing technologies like base editing.

Base editing is a precise genome editing technology that enables irreversible conversion of one target nucleotide into another in a programmable manner, without requiring a DSB or a donor template. The emerging base editing technologies currently comprise C-to-T base editors, A-to-G base editors, and C-to-G base editors. Currently, most of the cytidine deaminases used in C-to-T base editors are sourced from mammals and require a relatively high temperature (e.g., 37° C.) for optimal activity. However, base editing in plants and many animals is done at a lower temperature (e.g., 20° C. to 25° C.). Therefore, there is a need in the art for cytidine deaminases that can efficiently perform base editing at these lower temperatures. Furthermore, there is a need for cytidine deaminases that have editing windows and profiles different from those of cytidine deaminases discovered to date.

### SUMMARY

The presently disclosed subject matter relates generally to base editors useful for genome editing. Such base editors convert C to T in cells at high efficiency and with low levels of indels and non-C-to-T substitutions. In addition to the improved editing efficiency and precision, the base editors further differ from established base editors in terms of their editing windows.

Base editing fusion polypeptides are provided. The fusion polypeptides comprise: (i) a cytidine deaminase domain comprising an amino acid sequence having at least 80%, at least 90%, at least 95%, at least 98%, or at least 99% sequence identity to the amino acid sequence set forth in SEQ ID NO: 2, 6, 10, 14, 16, 17, 31, 37-40, 43-51, 53, 54, 56, 58-61, 63, or 64; and (ii) an RNA-guided DNA binding domain. In certain embodiments, the RNA-guided DNA binding domain comprises a Cas9 domain, a Cas12a domain, or a Cas12b domain. In certain embodiments, the RNA-guided DNA binding domain is nuclease active, nuclease inactive, or a nickase. In certain embodiments, the fusion polypeptide further comprises a uracil glycosylase inhibitor (UGI) domain. In certain embodiments, the fusion polypeptide further comprises a nuclear localization signal (NLS).

Cells and organisms, including plant cells and plants, comprising the fusion polypeptides, polynucleotides encoding fusion polypeptides, and vectors comprising the polynucleotides are also provided.

Methods of modifying a target nucleic acid are provided. The method comprise contacting the target nucleic acid with: (a) a fusion polypeptide comprising: (i) a cytidine deaminase domain comprising an amino acid sequence having at least 80%, at least 90%, at least 95%, at least 98%, or at least 99% sequence identity to the amino acid sequence set forth in SEQ ID NO: 2, 6, 10, 14, 16, 17, 31, 37-40, 43-51, 53, 54, 56, 58-61, 63, or 64, and (ii) an RNA-guided DNA binding domain; and (b) a DNA-targeting RNA, wherein the DNA-targeting RNA is capable of forming a complex with the RNA-guided DNA binding domain of the fusion polypeptide and directing the complex to the target nucleic acid, resulting in one or more C to T substitutions.

Methods for producing a genetically modified plant are also provided. The methods comprise introducing into the plant a fusion polypeptide comprising any of the cytidine deaminase disclosed herein, or a polynucleotide encoding the fusion polypeptide; and (b) a DNA-targeting RNA, or a DNA polynucleotide encoding the DNA-targeting RNA, wherein the DNA-targeting RNA is capable of forming a complex with the RNA-guided DNA binding domain of the fusion polypeptide and directing the complex to a target nucleic acid in the genome of the plant, resulting in one or more C to T substitutions.

While multiple embodiments are disclosed, still other embodiments of the present disclosure will become apparent based on the detailed description, which shows and describes illustrative embodiments of the disclosure. Accordingly, the figures and detailed description are to be regarded as illustrative in nature and not restrictive.

### BRIEF DESCRIPTION OF THE FIGURES

The following drawings form part of the specification and are included to further demonstrate certain example embodiments or various aspects of the invention. In some instances, example embodiments can be best understood by referring to the accompanying figures in combination with the detailed description presented herein. The description and accompanying figures may highlight a certain specific example, or a certain aspect of the invention. However, one skilled in the art will understand that portions of the examples or aspects provided in the present disclosure may be used in combination with other examples or aspects of the invention.

FIG. 1A-G shows evaluating novel cytidine deaminases coupled to BE3 architecture for base editing in rice protoplasts at the OsCGRS55 target site. FIG. 1A is a schematic of BE3 architecture of cytosine base editor. FIG. 1B shows the target site (bold) located in the fourth chromosome within OsCGRS55 followed by the PAM (underlined) (SEQ ID NO: 147). FIG. 1C shows preliminary testing of novel cytidine deaminases included in the first batch. Three biological replicates of rice protoplast assay were mixed together and PCR amplicon of the target site within OsCGRS55 gene was validated for C to T transition using NGS and CRISPRMatch software. PmCDA1, hAID, and hA3A/Y130F, indicated by dashed rectangle, represent broadly used cytidine deaminases for base editing in plants. FIG. 1D shows preliminary testing of novel cytidine deaminases included in the second batch. Three biological replicates of rice protoplast assay were mixed and PCR amplicon of the target site within OsCGRS55 gene was validated for C to T transition using NGS. PmCDA1, hAID, and hA3A-Y130F, highlighted in dashed rectangle, represent broadly used cytidine deaminases for base editing in plants. FIG. 1E shows testing of 29 best performing novel deaminases from the first and second batches for C-to-T conversions. OsCGRS55 target site was PCR amplified and validated for C-to-T transition using NGS and CRISPRMatch software. Depicted are three biological replicates, performed each on one of the three consecutive days. Error bars represent standard deviation. FIG. 1F shows testing of 29 best performing novel deaminases from two batches for C-to-A conversions. OsCGRS55 target site was PCR amplified and validated for C-to-A transition using NGS and CRISPRMatch software. Depicted are three biological replicates, performed each on one of the three consecutive days. Error bars represent standard deviation. FIG. 1G shows testing of 29 best performing novel deaminases from both batches for C-to-G conversions. OsCGRS55 target site was PCR amplified and validated for C-to-G transition using NGS and CRISPRMatch software. Depicted are three biological replicates, performed each on one of the three consecutive days. Error bars represent standard deviation.

FIG. 2A-R shows the activity windows of best performing base editors in rice protoplasts. OsCGRS55 target site was PCR amplified and validated for C-to-T transition using NGS and CRISPRMatch software. Activity windows were calculated from C-to-T transition frequency of the individual cytosines withing the OsCGRS55 target site compared to only edited sequences. Depicted are three biological replicates, performed each on one of the three consecutive days. Error bars represent standard deviation.

FIG. 3A-I shows evaluating novel cytidine deaminases coupled to BE3 architecture for base editing in tomato protoplasts. Two target sites in tomato were PCR amplified and validated for base editing/deletion introductions using NGS and CRISPRMatch software. Depicted are three biological replicates, error bars represent standard deviation. Established/current base editors are indicated by the dashed rectangles. FIG. 3A shows the two target sites (bold) located in the first chromosome within SolyAgo7 gene in tomato followed by the PAM (underlined) (SEQ ID NOs: 148 and 149). FIG. 3B shows testing of 16 best performing novel deaminases from the first and second batches for C-to-T conversions at SolyAgo7-gRNA3 target site. FIG. 3C shows testing 16 of best performing novel deaminases from the first and second batches for C-to-T conversions at SolyAgo7-gRNA4 target site. FIG. 3D shows testing 16 of best performing novel deaminases from the first and second batches for C-to-A conversions at SolyAgo7-gRNA3 target site. FIG. 3E shows testing 16 of best performing novel deaminases from the first and second batches for C-to-A conversions at SolyAgo7-gRNA4 target site. FIG. 3F shows testing 16 of best performing novel deaminases from the first and second batches for C-to-G conversions at SolyAgo7-gRNA3 target site. FIG. 3G shows testing 16 of best performing novel deaminases from the first and second batches for C-to-G conversions at SolyAgo7-gRNA4 target site. FIG. 3H shows testing 16 of best performing novel deaminases from the first and second batches for deletion introduction at SolyAgo7-gRNA3 target site. FIG. 3I shows testing 16 of best performing novel deaminases from the first and second batches for deletion introduction at SolyAgo7-gRNA4 target site.

FIG. 4A-T shows the activity windows of base editors in tomato protoplasts at SolyAgo7-gRNA3 target site. The SolyAgo7-gRNA3 target site was PCR amplified and validated for C-to-T transition using NGS and CRISPRMatch software. Activity windows were calculated from C-to-T transition frequency of the individual cytosines within SolyAgo7-gRNA3 target site in tomato compared to all amplified sequences (edited and not edited). Depicted are three biological replicates, error bars represent standard deviation. Error bars represent standard deviation.

FIG. 5A-T shows the activity windows of base editors in tomato protoplasts at SolyAgo7-gRNA4 target site. The SolyAgo7-gRNA4 target site was PCR amplified and validated for C-to-T transition using NGS and CRISPRMatch software. Activity windows were calculated from C-to-T transition frequency of the individual cytosines within SolyAgo7-gRNA4 target site in tomato compared to all amplified sequences (edited and not edited). Depicted are three biological replicates, error bars represent standard deviation. Error bars represent standard deviation.

### DETAILED DESCRIPTION

Unless defined otherwise, all technical and scientific terms used herein have the same meaning as commonly understood by one skilled in the art to which embodiments of the disclosure pertain. Many methods and materials similar, modified, or equivalent to those described herein can be used in the practice of the embodiments of the present disclosure without undue experimentation, the preferred materials and methods are described herein.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicates otherwise. The word “or” means any one member of a particular list and also includes any combination of members of that list. Further, all units, prefixes, and symbols may be denoted in their SI accepted form.

Numeric ranges recited within the specification are inclusive of the numbers defining the range and include each integer within the defined range. Throughout this disclosure, various aspects are presented in a range format. It should be understood that the description in range format is merely for convenience and brevity and should not be construed as an inflexible limitation on the scope of the present disclosure or the associated claims. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

The methods, systems, and compositions of the present disclosure may comprise, consist essentially of, or consist of the components described herein. As used herein, “consisting essentially of” means that the methods, systems, and compositions may include additional steps or components, but only if the additional steps or components do not materially alter the basic and novel characteristics of the claimed methods, systems, and compositions.

The term “CRISPR/Cas” or “clustered regularly interspaced short palindromic repeats” or “CRISPR” refers to DNA loci containing short repetitions of base sequences followed by short segments of spacer DNA from previous exposures to a virus or plasmid. Bacteria and archaea have evolved adaptive immune defenses termed CRISPR/CRISPR-associated (Cas) systems that use short RNA to direct degradation of foreign nucleic acids. In bacteria, the CRISPR system provides acquired immunity against invading foreign DNA via RNA-guided DNA cleavage.

The “CRISPR/Cas9” system or “CRISPR/Cas9-mediated gene editing” refers to a type II CRISPR/Cas system that has been modified for genome editing/engineering. It is typically comprised of a “guide” RNA (gRNA) and a non-specific CRISPR-associated endonuclease (Cas9). “Guide RNA (gRNA)” is used interchangeably herein with “short guide RNA (sgRNA)” or “single guide RNA (sgRNA). The sgRNA is a short synthetic RNA composed of a “scaffold” sequence necessary for Cas9-binding and a user-defined approximately 20 nucleotide “spacer” or “targeting” sequence which defines the genomic target to be modified. The genomic target of Cas9 can be changed by changing the targeting sequence present in the sgRNA.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “exogenous” refers to any material introduced from or produced outside an organism, cell, tissue or system.

The term “expression” as used herein is defined as the transcription and/or translation of a particular nucleotide sequence driven by its promoter.

As used herein, the term “heterologous” refers to a polynucleotide that originates from a foreign species, or, if from the same species, is modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

“Isolated” means altered or removed from the natural state. For example, a nucleic acid or a peptide naturally present in a living plant is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

As used herein, the term “stable transformation” is intended that a polynucleotide introduced into a plant integrates into the genome of the plant and is capable of being inherited by progeny thereof. As used herein, the term “transient transformation” is intended that a polynucleotide introduced into a plant does not integrate into the genome of the plant.

The terms “uracil glycosylase inhibitor” or “UGI” as used herein refer to a protein that is capable of inhibiting a uracil-DNA glycosylase base-excision repair enzyme.

A “vector” is a composition of matter which comprises an isolated nucleic acid and which can be used to deliver the isolated nucleic acid to the interior of a cell.

### Fusion Polypeptides

Fusion polypeptides containing a cytidine deaminase portion and a DNA binding (e.g., Cas9) portion are provided herein. As used herein, a “polypeptide” is an amino acid sequence including a plurality of consecutive polymerized amino acid residues (e.g. at least about 15 consecutive polymerized amino acid residues). “Polypeptide” refers to an amino acid sequence, oligopeptide, peptide, protein, or portions thereof, and the terms “polypeptide” and “protein” are used interchangeably.

Polypeptides as described herein also include polypeptides having various amino acid additions, deletions, or substitutions relative to the native amino acid sequence of a polypeptide of the present disclosure. In some embodiments, polypeptides that are homologs of a polypeptide of the present disclosure contain non-conservative changes of certain amino acids relative to the native sequence of a polypeptide of the present disclosure. In some embodiments, polypeptides that are homologs of a polypeptide of the present disclosure contain conservative changes of certain amino acids relative to the native sequence of a polypeptide of the present disclosure, and thus may be referred to as conservatively modified variants. A conservatively modified variant may include individual substitutions, deletions or additions to a polypeptide sequence which result in the substitution of an amino acid with a chemically similar amino acid. Conservative substitution tables providing functionally similar amino acids are well-known in the art. Such conservatively modified variants are in addition to and do not exclude polymorphic variants, interspecies homologs, and alleles of the disclosure. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). A modification of an amino acid to produce a chemically similar amino acid may be referred to as an analogous amino acid.

Fusion polypeptides of the present disclosure that are composed of individual polypeptide domains may be described based on the individual polypeptide domains of the overall fusion polypeptide. A domain in such a fusion polypeptide refers to the particular stretches of contiguous amino acid sequences with a particular function or activity. For example, a fusion polypeptide that is a fusion of a cytidine deaminase polypeptide and a DNA binding polypeptide, the contiguous amino acids that encode the cytidine deaminase polypeptide may be described as the cytidine deaminase domain in the overall fusion polypeptide, and the contiguous amino acids that encode the DNA binding polypeptide may be described as the DNA binding domain in the overall fusion polypeptide. Individual domains in an overall fusion protein may also be referred to as units of the fusion protein.

Certain embodiments of the present disclosure relate to a polypeptide comprising a cytidine deaminase domain and a DNA binding domain. In certain embodiments, the cytidine deaminase domain is recombinantly fused to a DNA binding domain (e.g., a cytidine deaminase-DNA binding fusion polypeptide). The cytidine deaminase domain may be in an N-terminal orientation or a C-terminal orientation relative to the DNA binding domain. The DNA binding domain may be in an N-terminal orientation or a C-terminal orientation relative to the cytidine deaminase domain. In some embodiments, a cytidine deaminase-DNA binding fusion protein may be a direct fusion of a cytidine deaminase domain and a DNA binding domain. In some embodiments, a cytidine deaminase-DNA binding fusion protein may be an indirect fusion of a cytidine deaminase domain and a DNA binding domain. In embodiments where the fusion is indirect, a linker domain or other contiguous amino acid sequence may separate the cytidine deaminase domain and the DNA binding domain.

### Cytidine Deaminases

The fusion polypeptides provided herein comprise a cytidine deaminase domain. As used herein, a “cytidine deaminase” refers to an enzyme that catalyzes the removal of an amine group from cytidine (i.e., the base cytosine when attached to a ribose ring) to uridine (C to U) and deoxycytidine to deoxyuridine (C to U). In general, a cytidine deaminase domain fused with an RNA-guided DNA binding domain (e.g., Cas9) can target a nucleic acid through the direction of a guide RNA to perform base editing, including the introduction of C to T substitutions.

In some embodiments, the cytidine deaminase is an apolipoprotein B mRNA-editing complex (APOBEC) family cytidine deaminase. In some embodiments, the cytidine deaminase is an APOBEC1 deaminase. In some embodiments, the cytidine deaminase is an APOBEC2 deaminase. In some embodiments, the cytidine deaminase is an APOBEC3 deaminase. In some embodiments, the cytidine deaminase is an APOBEC3A deaminase. In some embodiments, the cytidine deaminase is an APOBEC3B deaminase. In some embodiments, the cytidine deaminase is an APOBEC3C deaminase. In some embodiments, the cytidine deaminase is an APOBEC3D deaminase. In some embodiments, the cytidine deaminase is an APOBEC3E deaminase. In some embodiments, the cytidine deaminase is an APOBEC3F deaminase. In some embodiments, the cytidine deaminase is an APOBEC3G deaminase. In some embodiments, the cytidine deaminase is an APOBEC3H deaminase. In some embodiments, the cytidine deaminase is an APOBEC4 deaminase. In some embodiments, the cytidine deaminase is an activation-induced deaminase (AID). In some embodiments, the cytidine deaminase is a cytidine deaminase 1 (CDA1).

Examples of amino acid and nucleotide sequences of cytidine deaminases of the present disclosure are provided in Tables 1 and 2.

TABLE 1

| | | | Amino acid | |
|---|---|---|---|---|
| Name | Species | NCBI Accession | sequence | Coding sequence |
| AbA | Acinetobacter | WP_143213723 | SEQ ID NO: 1 | SEQ ID NO: 67 |
| | baumannii | | | |
| AmA1X1 | Alligator | XP_019337862 | SEQ ID NO: 2 | SEQ ID NO: 68 |
| | mississippiensis | | | |
| AsA2 | Alligator | XP_006025139 | SEQ ID NO: 3 | SEQ ID NO: 69 |
| | sinensis | | | |
| AsAID | Alligator | XP_006032700 | SEQ ID NO: 4 | SEQ ID NO: 70 |
| | sinensis | | | |
| AcA1 | Anolis | XP_008102031 | SEQ ID NO: 5 | SEQ ID NO: 71 |
| | carolinensis | | | |
| BasA3G | Balaenoptera | XP_007189136 | SEQ ID NO: 6 | SEQ ID NO: 72 |
| | acutorostrata | | | |
| | scammoni | | | |
| BpA2 | Boleophthalmus | XP_020791572 | SEQ ID NO: 7 | SEQ ID NO: 73 |
| | pectinirostris | | | |
| CmA2 | Chelonia mydas | XP_007063573 | SEQ ID NO: 8 | SEQ ID NO: 74 |
| CpbA1X11 | Chrysemys picta | XP_023965938 | SEQ ID NO: 9 | SEQ ID NO: 75 |
| | bellii | | | |
| CpAID | Crocodylus | XP_019399695 | SEQ ID NO: 10 | SEQ ID NO: 76 |
| | porosus | | | |
| CpA2 | Crocodylus | XP_019402241 | SEQ ID NO: 11 | SEQ ID NO: 77 |
| | porosus | | | |
| CsA2 | Cynoglossus | XP_008319408 | SEQ ID NO: 12 | SEQ ID NO: 78 |
| | semilaevis | | | |
| CcAID | Cyprinus carpio | XP_018981523 | SEQ ID NO: 13 | SEQ ID NO: 79 |
| DrAID | Danio rerio | NP_001008403 | SEQ ID NO: 14 | SEQ ID NO: 80 |
| DrA2A | Danio rerio | NP_001013332 | SEQ ID NO: 15 | SEQ ID NO: 81 |
| DnA3X2 | Dasypus | XP_004447910 | SEQ ID NO: 16 | SEQ ID NO: 82 |
| | novemcinctus | | | |
| DcAID | Denticeps | XP_028834826 | SEQ ID NO: 17 | SEQ ID NO: 83 |
| | clupeoides | | | |
| GgA1 | Gavialis | XP_019371538 | SEQ ID NO: 18 | SEQ ID NO: 84 |
| | gangeticus | | | |
| LoAID | Lagenorhynchus | XP_026948891 | SEQ ID NO: 19 | SEQ ID NO: 85 |
| | obliquidens | | | |
| LcAID | Latimeria | XP_014350178 | SEQ ID NO: 20 | SEQ ID NO: 86 |
| | chalumnae | | | |
| LvA1 | Lipotes vexillifer | XP_007469425 | SEQ ID NO: 21 | SEQ ID NO: 87 |
| NpAID | Nanorana | XP_018426850 | SEQ ID NO: 22 | SEQ ID NO: 88 |
| | parkeri | | | |
| NaaA3AX3 | Neophocaena | XP_024617344 | SEQ ID NO: 23 | SEQ ID NO: 89 |
| | asiaeorientalis | | | |
| | asiaeorientalis | | | |
| OoAID | Orcinus orca | XP_012391905 | SEQ ID NO: 24 | SEQ ID NO: 90 |
| PkA2 | Paramormyrops | XP_023669168 | SEQ ID NO: 25 | SEQ ID NO: 91 |
| | kingsleyae | | | |
| PsAID | Pelodiscus | XP_025038798 | SEQ ID NO: 26 | SEQ ID NO: 92 |
| | sinensis | | | |
| PvAID | Pogona vitticeps | XP_020670354 | SEQ ID NO: 27 | SEQ ID NO: 93 |
| PmA2 | Protobothrops | XP_015671004 | SEQ ID NO: 28 | SEQ ID NO: 94 |
| | mucrosquamatus | | | |
| PbAID | Python bivittatus | XP_025022614 | SEQ ID NO: 29 | SEQ ID NO: 95 |
| RtA2 | Rhincodon typus | XP_020366596 | SEQ ID NO: 30 | SEQ ID NO: 96 |
| RaA3A | Rousettus | XP_016017136 | SEQ ID NO: 31 | SEQ ID NO: 97 |
| | aegyptiacus | | | |
| SsCDA | Salmo salar | XP_014010073 | SEQ ID NO: 32 | SEQ ID NO: 98 |
| SaA2 | Salvelinus | XP_023842724 | SEQ ID NO: 33 | SEQ ID NO: 99 |
| | alpinus | | | |
| XlAID | Xenopus laevis | NP_001089181 | SEQ ID NO: 34 | SEQ ID NO: 100 |
| XlA2 | Xenopus laevis | XP_018104881 | SEQ ID NO: 35 | SEQ ID NO: 101 |
| XtA1 | Xenopus | XP_002941248 | SEQ ID NO: 36 | SEQ ID NO: 102 |
| | tropicalis | | | |

TABLE 2

| | | | Amino acid | |
|---|---|---|---|---|
| Name | Species | NCBI Accession | sequence | Coding sequence |
| BbbA3AX2 | Bison bison | XP_010843425.1 | SEQ ID NO: 37 | SEQ ID NO: 103 |
| | bison | | | |
| CdA3G | Camelus | XP_031319156.1 | SEQ ID NO: 38 | SEQ ID NO: 104 |
| | dromedarius | | | |
| CcA3X3 | Castor | XP_020029507.1 | SEQ ID NO: 39 | SEQ ID NO: 105 |
| | canadensis | | | |
| CsA3C | Chlorocebus | NP_001332881.1 | SEQ ID NO: 40 | SEQ ID NO: 106 |
| | sabaeus | | | |
| ClA3CX3 | Columba livia | XP_021153311.1 | SEQ ID NO: 41 | SEQ ID NO: 107 |
| DnA3X1 | Dasypus | XP_023445737.1 | SEQ ID NO: 42 | SEQ ID NO: 108 |
| | novemcinctus | | | |
| DlA3G | Delphinapterus | XP_022452168.1 | SEQ ID NO: 43 | SEQ ID NO: 109 |
| | leucas | | | |
| DrA3F | Desmodus | XP_024435233.1 | SEQ ID NO: 44 | SEQ ID NO: 110 |
| | rotundus | | | |
| EtA3C | Echinops | XP_030741606.1 | SEQ ID NO: 45 | SEQ ID NO: 111 |
| | telfairi | | | |
| EtA3A | Echinops | XP_030741607.1 | SEQ ID NO: 46 | SEQ ID NO: 112 |
| | telfairi | | | |
| EeA3 | Elephantulus | XP_006890259.1 | SEQ ID NO: 47 | SEQ ID NO: 113 |
| | edwardii | | | |
| EfA3CX1 | Eptesicus fuscus | XP_008159951.2 | SEQ ID NO: 48 | SEQ ID NO: 114 |
| EcA3HX1 | Equus caballus | XP_005606535.1 | SEQ ID NO: 49 | SEQ ID NO: 115 |
| EcA3G | Equus caballus | XP_023486964.1 | SEQ ID NO: 50 | SEQ ID NO: 116 |
| GmA3AX2 | Globicephala | XP_030711791.1 | SEQ ID NO: 51 | SEQ ID NO: 117 |
| | melas | | | |
| HmA3B | Hylobates | XP_032004340.1 | SEQ ID NO: 52 | SEQ ID NO: 118 |
| | moloch | | | |
| LoA3GX1 | Lagenorhynchus | XP_026963494.1 | SEQ ID NO: 53 | SEQ ID NO: 119 |
| | obliquidens | | | |
| LwA3HX1 | Leptonychotes | XP_030741607.1 | SEQ ID NO: 54 | SEQ ID NO: 120 |
| | weddellii | | | |
| LcA3F | Lontra | XP_032707103.1 | SEQ ID NO: 55 | SEQ ID NO: 121 |
| | canadensis | | | |
| LaA3GX2 | Loxodonta | XP_023415683.1 | SEQ ID NO: 56 | SEQ ID NO: 122 |
| | africana | | | |
| MlA3HX1 | Mandrillus | XP_011834225.1 | SEQ ID NO: 57 | SEQ ID NO: 123 |
| | leucophaeus | | | |
| MlA3C | Mandrillus | XP_011834235.1 | SEQ ID NO: 58 | SEQ ID NO: 124 |
| | leucophaeus | | | |
| MmA3AX1 | Monodon | XP_029060932.1 | SEQ ID NO: 59 | SEQ ID NO: 125 |
| | monoceros | | | |
| NaaA3AX2 | Neophocaena | XP_024617343.1 | SEQ ID NO: 60 | SEQ ID NO: 126 |
| | asiaeorientalis | | | |
| | asiaeorientalis | | | |
| OoA3GX2 | Orcinus orca | XP_012392105.1 | SEQ ID NO: 61 | SEQ ID NO: 127 |
| PaA3HX3 | Papio anubis | XP_003905607.2 | SEQ ID NO: 62 | SEQ ID NO: 128 |
| PsA3GX1 | Phocoena sinus | XP_032501203.1 | SEQ ID NO: 63 | SEQ ID NO: 129 |
| PcA3AX1 | Physeter | XP_028346448.1 | SEQ ID NO: 64 | SEQ ID NO: 130 |
| | catodon | | | |
| PvA3A | Pteropus | XP_011384362.1 | SEQ ID NO: 65 | SEQ ID NO: 131 |
| | vampyrus | | | |
| TmlA3B | Trichechus | XP_023584683.1 | SEQ ID NO: 66 | SEQ ID NO: 132 |
| | manatus | | | |
| | latirostris | | | |

In some embodiments, the cytidine deaminase is an American alligator, common minke whale, Australian saltwater crocodile, zebrafish, nine-banded armadillo, denticle herring, Egyptian rousette, American bison, Arabian camel, American beaver, green monkey, beluga whale, common vampire bat, small Madagascar hedgehog, Cape elephant shrew, big brown bat, horse, long-finned pilot whale, Pacific white-sided dolphin, Weddell seal, African bush elephant, drill, narwhal, Yangtze finless porpoise, orca, vaquita, or sperm whale deaminase.

In some embodiments, the cytidine deaminase is at least 80%, at least 85%, at least 90%, at least 92%, at least 95%, at least 96%, at least 97%, at least 98%, at least 99%, or at least 99.5% identical to any one of the amino acid sequences set forth in SEQ ID NOs: 1-66. In some embodiments, the cytidine deaminase comprises the amino acid sequence of any one of SEQ ID NOs: 1-66.

In some embodiments, the cytidine deaminase is at least 80%, at least 85%, at least 90%, at least 92%, at least 95%, at least 96%, at least 97%, at least 98%, at least 99%, or at least 99.5% identical to any one of the amino acid sequences set forth in SEQ ID NOs: SEQ ID NO: 2, 6, 10, 14, 16, 17, 31, 37-40, 43-51, 53, 54, 56, 58-61, 63, or 64. In some embodiments, the cytidine deaminase comprises the amino acid sequence of any one of SEQ ID NO: 2, 6, 10, 14, 16, 17, 31, 37-40, 43-51, 53, 54, 56, 58-61, 63, or 64.

In some embodiments, the cytidine deaminase is at least 80%, at least 85%, at least 90%, at least 92%, at least 95%, at least 96%, at least 97%, at least 98%, at least 99%, or at least 99.5% identical to any one of the amino acid sequences set forth in SEQ ID NOs: 2, 6, 14, 16, 31, 37, 38, 43, 45, 51, 53, 56, 59-61, or 63. In some embodiments, the cytidine deaminase comprises the amino acid sequence of any one of SEQ ID NOs: 2, 6, 14, 16, 31, 37, 38, 43, 45, 51, 53, 56, 59-61, or 63.

In some embodiments, the cytidine deaminase is at least 80%, at least 85%, at least 90%, at least 92%, at least 95%, at least 96%, at least 97%, at least 98%, at least 99%, or at least 99.5% identical to any one of the amino acid sequences set forth in SEQ ID NOs: 6, 16, 37, 38, 45, 53, or 59. In some embodiments, the cytidine deaminase comprises the amino acid sequence of any one of SEQ ID NOs: 6, 16, 37, 38, 45, 53, or 59.

In some embodiments, the cytidine deaminase is a common minke whale APOBEC3G deaminase comprising the amino acid sequence set forth in SEQ ID NO: 6. In some embodiments, the cytidine deaminase is a nine-banded armadillo APOBEC3 deaminase comprising the amino acid sequence set forth in SEQ ID NO: 16. In some embodiments, the cytidine deaminase is an American bison APOBEC3A deaminase comprising the amino acid sequence set forth in SEQ ID NO: 37. In some embodiments, the cytidine deaminase is an Arabian camel APOBEC3G deaminase comprising the amino acid sequence set forth in SEQ ID NO: 38. In some embodiments, the cytidine deaminase is a small Madagascar hedgehog APOBEC3C deaminase comprising the amino acid sequence set forth in SEQ ID NO: 45. In some embodiments, the cytidine deaminase is Pacific white-sided dolphin APOBEC3G deaminase comprising the amino acid sequence set forth in SEQ ID NO: 53. In some embodiments, the cytidine deaminase is a narwhal APOBEC3A deaminase comprising the amino acid sequence set forth in SEQ ID NO: 59.

In certain embodiments, the cytidine deaminase of the present disclosure has a broad deamination window in plant cells, for example, a deamination window with a length of at least 14 nucleotides, at least 15 nucleotides, or at least 16 nucleotides (e.g., C1 to C16, C2 to C16, C3 to C16). In some embodiments, one or more C bases within positions 1 to 16 of the target sequence are substituted with Ts. For example, if present, any one, two, three, four, five, six, seven, eight, nine, ten, eleven, twelve, thirteen, fourteen, fifteen, or sixteen Cs within positions 1 to 16 in the target sequence can be replaced with Ts. Therefore, if there are multiple Cs in the target sequence, a variety of mutation combinations can be obtained. In certain other embodiments, the cytidine deaminase of the present disclosure has a very narrow deamination window in plant cells, for example, a deamination window with a length of 1 nucleotide (e.g., around the C10 position).

### DNA Binding Polypeptides

A variety of DNA binding polypeptides may be used in the compositions and methods of the present disclosure. In certain embodiments, the DNA binding polypeptide is an RNA-guided DNA binding polypeptide. The term “RNA-guided DNA binding polypeptide” refers to any protein that may associate (e.g., form a complex) with one or more nucleic acid molecules (i.e., which may broadly be referred to as a “DNA-targeting RNA” and includes, for example, guide RNA in the case of Cas systems) which direct the protein to localize to a specific target nucleotide sequence (e.g., a gene locus of a genome) that is complementary to the one or more nucleic acid molecules (or a portion or region thereof) associated with the protein, thereby causing the protein to bind to the nucleotide sequence at the specific target site. The term RNA-guided DNA binding polypeptide includes CRISPR Cas9 proteins, as well as Cas9 equivalents, homologs, orthologs, or paralogs, whether naturally occurring or non-naturally occurring, and may include a Cas9 equivalent from any type of CRISPR system (e.g., type II, V, VI), including Cas12a and Cas12b (type V CRISPR-Cas systems).

In some embodiments, the RNA-guided DNA binding polypeptide is a Cas moiety. In various embodiment, the Cas moiety is a S. pyogenes Cas9, which has been mostly widely used as a tool for genome engineering. This Cas9 protein is a large, multi-domain protein containing two distinct nuclease domains. Point mutations can be introduced into Cas9 to abolish nuclease activity, resulting in a dead Cas9 (dCas9) that still retains its ability to bind DNA in a sgRNA-programmed manner. In principle, when fused to another protein or domain (e.g., a cytidine deaminase), dCas9 can target that protein to virtually any DNA sequence simply by co-expression with an appropriate sgRNA.

In still other embodiments, the Cas moiety may include any CRISPR associated protein, including but not limited to, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2. Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A.

A Cas moiety may also be referred to as a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which is hereby incorporated by reference.

Cas9 and equivalents recognizes a short motif in the CRISPR repeat sequences (the PAM or protospacer adjacent motif) to help distinguish self versus non-self. As noted herein, Cas9 nuclease sequences and structures are well known to those of skill in the art (see, e.g., “Complete genome sequence of an M1 strain of Streptococcuspyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference).

The Cas moiety may include any suitable homologs and/or orthologs. Cas9 homologs and/or orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain, that is, the Cas9 is a nickase.

In various embodiments, the base editing fusion polypeptides may comprise a nuclease-inactivated Cas protein may interchangeably be referred to as a “dCas” or “dCas9” protein (for nuclease-“dead” Cas9). Methods for generating a Cas9 protein (or a fragment thereof) having an inactive DNA cleavage domain are known (See, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the INH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9.

In some embodiments, the Cas9 polypeptide is a SpCas9 polypeptide. SpCas9 polypeptides may contain an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of SEQ ID NO: 133. It should be appreciated that additional Cas9 proteins (e.g., a nuclease dead Cas9 (dCas9), a Cas9 nickase (nCas9), or a nuclease active Cas9), including variants and homologs thereof, are within the scope of this disclosure.

In certain embodiments, the DNA binding domain may comprise a zinc finger DNA binding domain. Typically, a zinc finger DNA-binding domain contains three to six individual zinc finger repeats and can recognize between 9 and 18 base pairs. Each zinc finger repeat typically includes approximately 30 amino acids and comprises a Ppa-fold stabilized by a zinc ion. Adjacent zinc finger repeats arranged in tandem are joined together by linker sequences. Various strategies have been developed to engineer zinc finger domains to bind desired sequences, including both “modular assembly” and selection strategies that employ either phage display or cellular selection systems (Pabo C O et al., “Design and Selection of Novel Cys2His2 Zinc Finger Proteins” Annu. Rev. Biochem. (2001) 70: 313-40). The most straightforward method to generate new zinc-finger DNA-binding domains is to combine smaller zinc-finger repeats of known specificity. The most common modular assembly process involves combining three separate zinc finger repeats that can each recognize a 3 base pair DNA sequence to generate a 3-finger array that can recognize a 9 base pair target site. Other procedures can utilize either 1-finger or 2-finger modules to generate zinc-finger arrays with six or more individual zinc finger repeats. Alternatively, selection methods have been used to generate zinc-finger DNA-binding domains capable of targeting desired sequences.

In certain embodiments, the DNA binding domain may comprise a transcription activator-like effector (TALE). TALEs are proteins that are secreted by Xanthomonas bacteria via their type III secretion system when they infect plants. TALE DNA-binding domains contain a repeated highly conserved 33-34 amino acid sequence with divergent 12th and 13th amino acids, which are highly variable and show a strong correlation with specific nucleotide recognition. The relationship between amino acid sequence and DNA recognition allows for the engineering of specific DNA-binding domains by selecting a combination of repeat segments containing the appropriate variable amino acids.

### Uracil Glycosylase Inhibitor

Some aspects of the disclosure relate to fusion polypeptides that comprise a uracil glycosylase inhibitor (UGI) domain. It should be understood that the use of a UGI domain may increase the editing efficiency of a nucleic acid editing domain that is capable of catalyzing a C to U change. For example, fusion polypeptides comprising a UGI domain may be more efficient in deaminating C residues.

In some embodiments, a UGI domain comprises a UGI as set forth in SEQ ID NO: 135. In some embodiments, the UGI comprises an amino acid sequence that is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to the UGI as set forth in SEQ ID NO: 135.

### Nuclear Localization Signals (NLS)

Fusion polypeptides of the present disclosure may contain one or more nuclear localization signals (NLS). Nuclear localization signals may also be referred to as nuclear localization sequences, domains, peptides, or other terms readily apparent to those of skill in the art. Nuclear localization signals are a translocation sequence that, when present in a polypeptide, direct that polypeptide to localize to the nucleus of a eukaryotic cell.

Various nuclear localization signals may be used in fusion polypeptides of the present disclosure. For example, one or more SV40-type NLS or one or more nucleoplasmin NLS may be used in fusion polypeptides. Fusion polypeptides may also contain two or more tandem copies of a nuclear localization signal. For example, fusion polypeptides may contain at least two, at least three, at least four, at least five, at least six, at least seven, at least eight, at least nine, or at least ten copies, either tandem or not, of a nuclear localization signal.

Fusion polypeptides of the present disclosure may contain one or more nuclear localization signals that contain an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of SEQ ID NO: 137 or 139.

### Tags, Reporters, and Other Features

Fusion polypeptides of the present disclosure may contain one or more tags that allow for e.g., purification and/or detection of the fusion polypeptide. Various tags may be used herein and are well-known to those of skill in the art. Exemplary tags may include HA, GST, FLAG, MBP, etc., and multiple copies of one or more tags may be present in a fusion polypeptide.

Fusion polypeptides of the present disclosure may contain one or more reporters that allow for e.g., visualization and/or detection of the fusion polypeptide. A reporter polypeptide encodes a protein that may be readily detectable due to its biochemical characteristics such as, for example, enzymatic activity or chemifluorescent features. Reporter polypeptides may be detected in a number of ways depending on the characteristics of the particular reporter. For example, a reporter polypeptide may be detected by its ability to generate a detectable signal (e.g., fluorescence), by its ability to form a detectable product, etc. Various reporters may be used herein and are well-known to those of skill in the art. Exemplary reporters may include GFP, GUS, mCherry, luciferase, etc., and multiple copies of one or more tags may be present in a fusion polypeptide.

Fusion polypeptides of the present disclosure may contain one or more polypeptide domains that serve a particular purpose depending on the particular goal/need. For example, fusion polypeptides may contain translocation sequences that target the polypeptide to a particular cellular compartment or area. Suitable features will be readily apparent to those of skill in the art.

### Linkers

In certain embodiments, linkers may be used to link any of the proteins or protein domains described herein. In general, linkers are short peptides that separate the different domains in a multi-domain protein. They may play an important role in fusion proteins, affecting the crosstalk between the different domains, the yield of protein production, and the stability and/or the activity of the fusion proteins. Linkers are generally classified into 2 major categories: flexible or rigid. Flexible linkers are typically used when the fused domains require a certain degree of movement or interaction, and these linkers are usually composed of small amino acids such as, for example, glycine (G), serine (S) or proline (P).

The certain degree of movement between domains allowed by flexible linkers is an advantage in some fusion proteins. However, it has been reported that flexible linkers can sometimes reduce protein activity due to an inefficient separation of the two domains. In this case, rigid linkers may be used since they enforce a fixed distance between domains and promote their independent functions. A thorough description of several linkers has been provided in Chen X et al., 2013, Advanced Drug Delivery Reviews 65 (2013) 1357-1369).

Various linkers may be used in, for example, the construction of fusion polypeptides as described herein. Linkers may be used in e.g., fusion proteins as described herein to separate the coding sequences of the cytidine deaminase domain and the Cas domain. For example, a variety of wiggly/flexible linkers, stiff/rigid linkers, short linkers, and long linkers may be used as described herein. Various linkers as described herein may be used in the construction of fusion proteins as described herein.

A variety of shorter or longer linker regions are known in the art, for example corresponding to a series of glycine residues, a series of adjacent glycine-serine dipeptides, a series of adjacent glycine-glycine-serine tripeptides, or known linkers from other proteins. In some embodiments, a linker comprises the amino acid sequence SGSETPGTSESATPES (SEQ ID NO: 141), which may also be referred to as the XTEN linker. In some embodiments, a linker comprises the amino acid sequence GDGSGGS (SEQ ID NO: 143). In some embodiments, a linker comprises the amino acid sequence SGGS (SEQ ID NO: 145). It should be appreciated that any of the linkers provided herein may be used to link a cytidine deaminase domain, an RNA-guided DNA binding domain, and a UGI domain in any of the fusion polypeptides provided herein.

### Nucleic Acids Encoding Fusion Polypeptides

Certain embodiments of the present disclosure relate to nucleic acids encoding fusion polypeptides of the present disclosure. Certain aspects of the present disclosure relate to nucleic acids encoding various portions/domains of fusion polypeptides of the present disclosure.

As used herein, the terms “polynucleotide,” “nucleic acid,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotide backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with analog and inter-nucleotide modifications. As used herein, the symbols for nucleotides and polynucleotides are those recommended by the IUPAC-IUB Commission of Biochemical Nomenclature.

Sequences of the polynucleotides of the present disclosure may be prepared by various suitable methods known in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those skilled in the art and is described in the pertinent texts and literature (e.g., in Matteucci et al., (1980) Tetrahedron Lett 21:719-722; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired polynucleotide sequence from the gel via techniques known to those skilled in the art, such as utilization of polymerase chain reactions (PCR; e.g., U.S. Pat. No. 4,683,195).

The nucleic acids employed in the methods and compositions described herein may be codon optimized relative to a parental template for expression in a particular host cell. Cells differ in their usage of particular codons, and codon bias corresponds to relative abundance of particular tRNAs in a given cell type. By altering codons in a sequence so that they are tailored to match with the relative abundance of corresponding tRNAs, it is possible to increase expression of a product (e.g., a polypeptide) from a nucleic acid. Similarly, it is possible to decrease expression by deliberately choosing codons corresponding to rare tRNAs. Thus, codon optimization/deoptimization can provide control over nucleic acid expression in a particular cell type (e.g., bacterial cell, plant cell, mammalian cell, etc.). Methods of codon optimizing a nucleic acid for tailored expression in a particular cell type are well-known to those of skill in the art.

Various methods are known to those of skill in the art for identifying similar (e.g. homologs, orthologs, paralogs, etc.) polypeptide and/or polynucleotide sequences, including phylogenetic methods, sequence similarity analysis, and hybridization methods.

Phylogenetic trees may be created for a gene family by using a program such as CLUSTAL (Thompson et al. Nucleic Acids Res. 22: 4673-4680 (1994); Higgins et al. Methods Enzymol 266: 383-402 (1996)) or MEGA (Tamura et al. Mol. Biol. & Evo. 24:1596-1599 (2007)). Once an initial tree for genes from one species is created, potential orthologous sequences can be placed in the phylogenetic tree and their relationships to genes from the species of interest can be determined. Evolutionary relationships may also be inferred using the Neighbor-Joining method (Saitou and Nei, Mol. Biol. & Evo. 4:406-425 (1987)). Homologous sequences may also be identified by a reciprocal BLAST strategy. Evolutionary distances may, for example, be computed using the Poisson correction method (Zuckerkandl and Pauling, pp. 97-166 in Evolving Genes and Proteins, edited by V. Bryson and H. J. Vogel. Academic Press, New York (1965)).

In addition, evolutionary information may be used to predict gene function. Functional predictions of genes can be greatly improved by focusing on how genes became similar in sequence (i.e., by evolutionary processes) rather than on the sequence similarity itself (Eisen, Genome Res. 8: 163-167 (1998)). Many specific examples exist in which gene function has been shown to correlate well with gene phylogeny (Eisen, Genome Res. 8: 163-167 (1998)). By using a phylogenetic analysis, one skilled in the art would recognize that the ability to deduce similar functions conferred by closely-related polypeptides is predictable.

When a group of related sequences are analyzed using a phylogenetic program such as CLUSTAL, closely related sequences typically cluster together or in the same clade (a group of similar genes). Groups of similar genes can also be identified with pair-wise BLAST analysis (Feng and Doolittle, J. Mol. Evol. 25: 351-360 (1987)). Analysis of groups of similar genes with similar functions that fall within one clade can yield sub-sequences that are particular to the clade. These sub-sequences, known as consensus sequences, can not only be used to define the sequences within each chide, but define the functions of these genes; genes within a clade may contain paralogous sequences, or orthologous sequences that share the same function (see also, for example, Mount, Bioinformatics: Sequence and Genome Analysis Cold Spring Harbor Laboratory Press, Cold Spring Harbor, N.Y., page 543 (2001)).

To find sequences that are homologous to a reference sequence, BLAST nucleotide searches can be performed with the BLASTN program, score=100, wordlength=12, to obtain nucleotide sequences homologous to a nucleotide sequence encoding a protein of the disclosure. BLAST protein searches can be performed with the BLASTX program, score=50, wordlength=3, to obtain amino acid sequences homologous to a protein or polypeptide of the disclosure. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, or PSI-BLAST, the default parameters of the respective programs (e.g., BLASTN for nucleotide sequences, BLASTX for proteins) can be used.

Methods for the alignment of sequences and for the analysis of similarity and identity of polypeptide and polynucleotide sequences are well-known in the art.

As used herein “sequence identity” refers to the percentage of residues that are identical in the same positions in the sequences being analyzed. As used herein “sequence similarity” refers to the percentage of residues that have similar biophysical/biochemical characteristics in the same positions (e.g., charge, size, hydrophobicity) in the sequences being analyzed.

Methods of alignment of sequences for comparison are well-known in the art, including manual alignment and computer assisted sequence alignment and analysis. This latter approach is a preferred approach in the present disclosure, due to the increased throughput afforded by computer-assisted methods. As noted below, a variety of computer programs for performing sequence alignment are available or can be produced by one of skill in the art.

The determination of percent sequence identity and/or similarity between any two sequences can be accomplished using a mathematical algorithm. Examples of such mathematical algorithms are the algorithm of Myers and Miller, CABIOS 4:11-17 (1988); the local homology algorithm of Smith et al., Adv. Appl. Math. 2:482 (1981); the homology alignment algorithm of Needleman and Wunsch, J. Mol. Biol. 48:443-453 (1970); the search-for-similarity-method of Pearson and Lipman, Proc. Natl. Acad. Sci. 85:2444-2448 (1988); the algorithm of Karlin and Altschul; Proc. Natl. Acad. Sci. USA 87:2264-2268 (1990), modified as in Karlin and Altschul, Proc. Natl. Acad. Sci. USA 90:5873-5877 (1993).

Computer implementations of these mathematical algorithms can be utilized for comparison of sequences to determine sequence identity and/or similarity. Such implementations include, for example: CLUSTAL in the PC/Gene program (available from Intelligenetics, Mountain View, Calif); the AlignX program, version10.3.0 (Invitrogen, Carlsbad, Calif.) and GAP, BESTFIT, BLAST, FASTA, and TFASTA in the Wisconsin Genetics Software Package, Version 8 (available from Genetics Computer Group (GCG), 575 Science Drive; Madison; Wis., USA). Alignments using these programs can be performed using the default parameters. The CLUSTAL program is well described by Higgins et al. Gene 73:237-244 (1988); Higgins et al. CABIOS 5:151-153 (1989); Corpet et al., Nucleic Acids Res. 16:10881-90 (1988); Huang et al. CABIOS 8:155-65 (1992); and Pearson et al., Meth. Mol. Biol. 24:307-331 (1994). The BLAST programs of Altschul et al. Mol. Biol. 215:403-410 (1990) are based on the algorithm of Karlin and Altschul (1990) supra.

Polynucleotides homologous to a reference sequence can be identified by hybridization to each other under stringent or under highly stringent conditions. Single-stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives; solvents, etc. present in both the hybridization and wash solutions and incubations (and number thereof), as described in more detail in references cited below (e.g., Sambrook et al., Molecular Cloning: A Laboratory Manual, 2nd Ed., Vol. 1-3, Cold Spring Harbor Laboratory, Cold Spring Harbor, N.Y. (“Sambrook”) (1989); Berger and Kimmel, Guide to Molecular Cloning Techniques, Methods in Enzymology, vol. 152 Academic Press, Inc., San Diego, Calif. (“Berger and Kimmel”) (1987); and Anderson and Young, “Quantitative Filter Hybridisation.” In: Flames and Higgins, ed., Nucleic Acid Hybridisation, A Practical Approach. Oxford; TRL Press, 73-111 (1985)).

Encompassed by the disclosure are polynucleotide sequences that are capable of hybridizing to the disclosed polynucleotide sequences and fragments thereof under various conditions of stringency (see, for example, Wahl and Berger, Methods Enzymol. 152: 399-407 (1987); and Kimmel, Methods Enzymo. 152: 507-511, (1987)). Full-length cDNA, homologs, orthologs, and paralogs of polynucleotides of the present disclosure may be identified and isolated using well-known polynucleotide hybridization methods.

With regard to hybridization, conditions that are highly stringent; and means for achieving them, are well known in the art. See, for example, Sambrook et al, (1989) (supra); Berger and Kimmel (1987) pp. 467-469 (supra); and Anderson and Young (1985)(supra).

Hybridization experiments are generally conducted in a buffer of pH between 6.8 to 7.4, although the rate of hybridization is nearly independent of pH at ionic strengths likely to be used in the hybridization buffer (Anderson and Young (1985) (supra)). In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments such as genes that duplicate functional enzymes from closely related organisms. The stringency can be adjusted either during the hybridization step or in the post-hybridization washes. Salt concentration, formamide concentration, hybridization temperature and probe lengths are variables that can be used to alter stringency. As a general guideline, high stringency is typically performed at Tₘ−5° C. to Tₘ−20° C., moderate stringency at Tₘ−20° C. to Tₘ−35° C. and low stringency at Tₘ−35° C. to Tₘ−50° C. for duplex >150 base pairs. Hybridization may be performed at low to moderate stringency (25-50° C. below Tₘ), followed by post-hybridization washes at increasing stringencies. Maximum rates of hybridization in solution are determined empirically to occur at Tₘ−25° C. for DNA-DNA duplex and Tₘ−15° C. for RNA-DNA duplex. Optionally, the degree of dissociation may be assessed after each wash step to determine the need for subsequent, higher stringency wash steps.

High stringency conditions may be used to select nucleic acid sequences with high degrees of identity to the disclosed sequences. An example of stringent hybridization conditions obtained in a filter-based method such as a Southern or northern blot for hybridization of complementary nucleic acids that have more than 100 complementary residues is about 5° C. to 20° C. lower than the thermal melting point (Tₘ) for the specific sequence at a defined ionic strength and pH.

Hybridization and wash conditions that may be used to bind and remove polynucleotides with less than the desired homology to the nucleic acid sequences or their complements of the present disclosure include, for example: 6×SSC and 1% SDS at 65° C.; 50% formamide, 4×SSC at 42° C.; 0.5×SSC to 2.0×SSC, 0.1% SDS at 50° C. to 65° C.; or 0.1×SSC to 2×SSC, 0.1% SDS at 50° C.-65° C.; with a first wash step of, for example, 10 minutes at about 42° C. with about 20% (v/v) formamide in 0.1×SSC, and with, for example, a subsequent wash step with 0.2×SSC and 0.1% SUS at 65° C. for 10, 20 or 30 minutes.

For identification of less closely related homologs, wash steps may be performed at a lower temperature, e.g., 50° C. An example of a low stringency wash step employs a solution and conditions of at least 25° C. in 30 mM NaCl, 3 mM trisodium citrate, and 0.1% SDS over 30 min. Greater stringency may be obtained at 42° C. in 15 mM NaCl, with 1.5 mM trisodium citrate, and 0.1% SDS over 30 min, Wash procedures will generally employ at least two final wash steps. Additional variations on these conditions will be readily apparent to those skilled in the art (see, for example, US Patent Application No. 20010010913).

If desired, one may employ wash steps of even greater stringency, including conditions of 65° C.-68° C. in a solution of 15 mM NaCl, 1.5 mM trisodium citrate, and 0.1% SDS, or about 0.2×SSC, 0.1% SDS at 65° C. and washing twice, each wash step of 10, 20 or 30 min in duration, or about 0.1×SSC, 0.1% SDS at 65° C. and washing twice for 10, 20 or 30 min. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C.

### Methods for Modifying a Nucleotide Sequence in a Genome

Methods are provided herein for modifying a nucleotide sequence of a genome. Non-limiting examples of genomes include cellular, nuclear, organellar, and plasmid genomes. The methods comprise introducing into a genome host (e.g., a cell or organelle) one or more DNA-targeting polynucleotides such as a DNA-targeting RNA (“guide RNA,” “gRNA,” “CRISPR RNA,” or “crRNA”) or a DNA polynucleotide encoding a DNA-targeting RNA, wherein the DNA-targeting polynucleotide comprises: (a) a first segment comprising a nucleotide sequence that is complementary to a sequence in the target DNA; and (b) a second segment that interacts with an RNA-guided DNA binding domain of a fusion polypeptide and also introducing to the genome host a fusion polypeptide, or a polynucleotide encoding a fusion polypeptide, wherein the fusion polypeptide comprises: (a) a polynucleotide-binding portion that interacts with the gRNA or other DNA-targeting polynucleotide; and (b) an cytidine deaminase portion. The genome host can then be cultured under conditions in which the fusion polypeptide is expressed. Finally, a genome host comprising the modified nucleotide sequence can be selected.

The methods disclosed herein comprise introducing into a genome host at least one fusion polypeptide or a nucleic acid encoding at least one fusion polypeptide, as described herein. In some embodiments, the fusion polypeptide can be introduced into the genome host as an isolated protein. In such embodiments, the fusion polypeptide can further comprise at least one cell-penetrating domain, which facilitates cellular uptake of the protein. In some embodiments, the fusion polypeptide can be introduced into the genome host as a nucleoprotein in complex with a guide polynucleotide (for instance, as a ribonucleoprotein in complex with a guide RNA). In other embodiments, the fusion polypeptide can be introduced into the genome host as an mRNA molecule that encodes the fusion polypeptide. In still other embodiments, the fusion polypeptide can be introduced into the genome host as a DNA molecule comprising an open reading frame that encodes the fusion polypeptide. In general, DNA sequences encoding the fusion polypeptide described herein are operably linked to a promoter sequence that will function in the genome host. The DNA sequence can be linear, or the DNA sequence can be part of a vector. In still other embodiments, the fusion polypeptide can be introduced into the genome host as an RNA-protein complex comprising the guide RNA.

In certain embodiments, mRNA encoding the fusion polypeptide may be targeted to an organelle (e.g., plastid or mitochondria). In certain embodiments, mRNA encoding one or more guide RNAs may be targeted to an organelle (e.g., plastid or mitochondria). In certain embodiments, mRNA encoding the fusion polypeptide and one or more guide RNAs may be targeted to an organelle (e.g., plastid or mitochondria). Methods for targeting mRNA to organelles are known in the art (see, e.g., U.S. Patent Application 2011/0296551; U.S. Patent Application 2011/0321187; Gómez and Pallás (2010) PLoS One 5:e12269), and are incorporated herein by reference.

In certain embodiments, DNA encoding the fusion polypeptide can further comprise a sequence encoding a guide RNA. In general, each of the sequences encoding the fusion polypeptide and the guide RNA is operably linked to one or more appropriate promoter control sequences that allow expression of the fusion polypeptide and the guide RNA, respectively, in the genome host. The DNA sequence encoding the fusion polypeptide and the guide RNA can further comprise additional expression control, regulatory, and/or processing sequence(s). The DNA sequence encoding the fusion polypeptide and the guide RNA can be linear or can be part of a vector.

Methods described herein further can also comprise introducing into a genome host at least one guide RNA or DNA encoding at least one polynucleotide such as a guide RNA. A guide RNA interacts with the RNA-guided DNA binding domain of the fusion polypeptide to direct the fusion polypeptide to a specific target site, at which site the guide RNA base pairs with a specific DNA sequence in the targeted site. Guide RNAs can comprise three regions: a first region that is complementary to the target site in the targeted DNA sequence, a second region that forms a stem loop structure, and a third region that remains essentially single-stranded. The first region of each guide RNA is different such that each guide RNA guides a fusion polypeptide to a specific target site. The second and third regions of each guide RNA can be the same in all guide RNAs.

One region of the guide RNA is complementary to a sequence (i.e., protospacer sequence) at the target site in the targeted DNA such that the first region of the guide RNA can base pair with the target site. In various embodiments, the first region of the guide RNA can comprise from about 8 nucleotides to more than about 30 nucleotides. For example, the region of base pairing between the first region of the guide RNA and the target site in the nucleotide sequence can be about 8, about 9, about 10, about 11, about 12, about 13, about 14, about 15, about 16, about 17, about 18, about 19, about 20, about 22, about 23, about 24, about 25, about 27, about 30 or more than 30 nucleotides in length. In an exemplary embodiment, the first region of the guide RNA is about 23, 24, or 25 nucleotides in length. The guide RNA also can comprise a second region that forms a secondary structure. In some embodiments, the secondary structure comprises a stem or hairpin. The length of the stem can vary. For example, the stem can range from about 5, to about 6, to about 10, to about 15, to about 20, to about 25 base pairs in length. The stem can comprise one or more bulges of 1 to about 10 nucleotides. The overall length of the second region can range from about 14 to about 25 nucleotides in length. In certain embodiments, the loop is about 3, 4, or 5 nucleotides in length and the stem comprises about 5, 6, 7, 8, 9, or 10 base pairs.

The guide RNA can also comprise a third region that remains essentially single-stranded. Thus, the third region has no complementarity to any nucleotide sequence in the cell of interest and has no complementarity to the rest of the guide RNA. The length of the third region can vary. In general, the third region is more than about 4 nucleotides in length. For example, the length of the third region can range from about 5 to about 60 nucleotides in length. The combined length of the second and third regions (also called the universal or scaffold region) of the guide RNA can range from about 30 to about 120 nucleotides in length. In one aspect, the combined length of the second and third regions of the guide RNA range from about 40 to about 45 nucleotides in length.

In some embodiments, the guide RNA comprises a single molecule comprising all three regions. In other embodiments, the guide RNA can comprise two separate molecules. The first RNA molecule can comprise the first region of the guide RNA and one half of the “stem” of the second region of the guide RNA. The second RNA molecule can comprise the other half of the “stem” of the second region of the guide RNA and the third region of the guide RNA. Thus, in this embodiment, the first and second RNA molecules each contain a sequence of nucleotides that are complementary to one another. For example, in one embodiment, the first and second RNA molecules each comprise a sequence (of about 6 to about 25 nucleotides) that base pairs to the other sequence to form a functional guide RNA.

In certain embodiments, the guide RNA can be introduced into the genome host as an RNA molecule. The RNA molecule can be transcribed in vitro. Alternatively, the RNA molecule can be chemically synthesized. In other embodiments, the guide RNA can be introduced into the genome host as a DNA molecule. In such cases, the DNA encoding the guide RNA can be operably linked to one or more promoter sequences for expression of the guide RNA in the genome host. For example, the RNA coding sequence can be operably linked to a promoter sequence that is recognized by RNA polymerase III (Pol III).

The DNA molecule encoding the guide RNA can be linear or circular. In some embodiments, the DNA sequence encoding the guide RNA can be part of a vector. Suitable vectors include plasmid vectors, phagemids, cosmids, artificial/mini-chromosomes, transposons, and viral vectors. In an exemplary embodiment, the DNA encoding the guide RNA is present in a plasmid vector. Non-limiting examples of suitable plasmid vectors include pUC, pBR322, pET, pBluescript, pCAMBIA, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like.

A variety of promoters may be used to drive expression of the guide RNA. Guide RNAs may be expressed using a Pol III promoter such as, for example, the U3 promoter, U6 promoter, or the H1 promoter (eLife 2013 2:e00471). For example, an approach in plants has been described using three different Pol III promoters from three different Arabidopsis U6 genes, and their corresponding gene terminators (BMC Plant Biology 2014 14:327). One skilled in the art would readily understand that many additional Pol III promoters could be utilized to simultaneously express many guide RNAs to many different locations in the genome. The use of different Pol III promoters for each gRNA expression cassette may be desirable to reduce the chances of natural gene silencing that can occur when multiple copies of identical sequences are expressed in plants. Alternatively, a tRNA-gRNA expression cassette (Xie, X et al, 2015, Proc Natl Acad Sci USA. 2015 Mar. 17; 112(11):3570-5) may be used to deliver multiple gRNAs simultaneously with high expression levels.

In embodiments in which both the fusion polypeptide and the guide RNA are introduced into the genome host as DNA molecules, each can be part of a separate molecule (e.g., one vector containing fusion polypeptide coding sequence and a second vector containing guide RNA coding sequence) or both can be part of the same molecule (e.g., one vector containing coding (and regulatory) sequence for both the fusion polypeptide and the guide RNA).

Various types of nucleic acids may be targeted for base editing as will be readily apparent to one of skill in the art. The target site can be in the coding region of a gene, in an intron of a gene, in a control region of a gene, in a non-coding region between genes, etc. The gene can be a protein coding gene or an RNA coding gene. The gene can be any gene of interest. The target nucleic acid may reside endogenously in a target gene or may be inserted into the gene, e.g., heterologous, for example, using techniques such as homologous recombination.

### Plants of the Present Disclosure

As used herein, a “plant” refers to any of various photosynthetic, eukaryotic multi-cellular organisms of the kingdom Plantae, characteristically producing embryos, containing chloroplasts, having cellulose cell walls and lacking locomotion. As used herein, a “plant” includes any plant or part of a plant at any stage of development, including seeds, suspension cultures, plant cells, embryos, meristematic regions, callus tissue, leaves, roots, shoots, gametophytes, sporophytes, pollen, microspores, and progeny thereof. Also included are cuttings, and cell or tissue cultures. As used in conjunction with the present disclosure, plant tissue includes, for example, whole plants, plant cells, plant organs, e.g., leaves, stems, roots, meristems, plant seeds, protoplasts, callus, cell cultures, and any groups of plant cells organized into structural and/or functional units.

Any plant cell may be used in the present disclosure. As disclosed herein, a broad range of plant types may be modified to incorporate fusion polypeptides and/or polynucleotides of the present disclosure. Suitable plants that may be modified include both monocotyledonous (monocot) plants and dicotyledonous (dicot) plants.

Examples of suitable plants may include, for example, species of the Family Gramineae, including Sorghum bicolor and Zea mays; species of the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Cichorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, and Triticum.

In some embodiments, plant cells may include, for example, those from corn (Zea mays), canola (Brassica napus, Brassica rapa ssp.), Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), duckweed (Lemna), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatas), cassava (Manihot esculenta), coffee (Coffea spp.), coconut (Cocos nucijra), pineapple (Ananas comosus), citrus trees (Citrus spp.), cocoa (Theobroma cacao), tea (Camellia sinensis), banana (Musa spp.), avocado (Persea americana), fig (Ficus carica), guava (Psidium guajava), mango (Mangifera indica), olive (Olea europaea), Papaya (Carica papaya), cashew (Anacardium occidentale), Macadamia (Macadamia spp.), almond (Prunus amygdalus), sugar beets (Beta vulgaris), sugarcane (Saccharum spp), oats, barley, vegetables, ornamentals, and conifers.

Examples of suitable vegetable plants may include, for example, tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo).

Examples of suitable ornamental plants may include, for example, azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Examples of suitable conifer plants may include, for example, loblolly pine (Pinus taeda), slash pine (Pinus elliottii), Ponderosa pine (Pinus ponderosa), lodgepole pine (Pinus contorta), Monterey pine (Pinus radiata), Douglas-fir (Pseudotsuga menziesii), Western hemlock (Tsuga canadensis), Sitka spruce (Picea glauca), redwood (Sequoia sempervirens), silver fir (Abies amabilis), balsam fir (Abies balsamea), Western red cedar (Thuja plicata), and Alaska yellow-cedar (Chamaecyparis nootkatensis).

Examples of suitable leguminous plants may include, for example, guar, locust bean, fenugreek, soybean, garden beans, cowpea, mungbean, lima bean, fava bean, lentils, chickpea, peanuts (Arachis sp.), crown vetch (Vicia sp.), hairy vetch, adzuki bean, lupine (Lupinus sp.), trifolium, common bean (Phaseolus sp.), field bean (Pisum sp.), clover (Melilotus sp.) Lotus, trefoil, lens, and false indigo.

Examples of suitable forage and turf grass may include, for example, alfalfa (Medicago ssp.), orchard grass, tall fescue, perennial ryegrass, creeping bentgrass, and redtop.

Examples of suitable crop plants and model plants may include, for example, Arabidopsis, corn, rice, alfalfa, sunflower, canola, soybean, cotton, peanut, sorghum, wheat, and tobacco.

### Expression of a Fusion Polypeptide in Plants

Fusion polypeptides of the present disclosure may be introduced into plant cells via any suitable methods known in the art. For example, a fusion polypeptide can be exogenously added to plant cells and the plant cells are maintained under conditions such that the fusion polypeptide is involved with targeting one or more target nucleic acids to activate the expression of the target nucleic acids in the plant cells. Alternatively, a nucleic acid encoding a fusion polypeptide of the present disclosure can be expressed in plant cells. Additionally, in some embodiments, a fusion polypeptide of the present disclosure may be transiently expressed in a plant via viral infection of the plant. Methods of introducing proteins via viral infection or via the introduction of RNAs into plants are well known in the art. For example, Tobacco Rattle Virus (TRV) has been successfully used to introduce zinc finger nucleases in plants to cause genome modification (“Nontransgenic Genome Modification in Plant Cells”, Plant Physiology 154:1079-1087 (2010)).

A nucleic acid encoding a fusion polypeptide of the present disclosure can be expressed in a plant with any suitable plant expression vector. Typical vectors useful for expression of nucleic acids in higher plants are well known in the art and include, for example, vectors derived from the tumor-inducing (Ti) plasmid of Agrobacterium tumefaciens (e.g., see Rogers et al., Meth. in Enzymol. (1987) 153:253-277). These vectors are plant integrating vectors in that on transformation, the vectors integrate a portion of vector DNA into the genome of the host plant. Exemplary A. tumefaciens vectors useful herein are plasmids pKYLX6 and pKYLX7 (e.g., see of Schardl et al., Gene (1987) 61:1-11; and Berger et al., Proc. Natl. Acad. Sci. USA (1989) 86:8402-8406); and plasmid pBI 101.2 that is available from Clontech Laboratories, Inc. (Palo Alto, Calif).

In addition to regulatory domains, fusion polypeptides of the present disclosure can be coupled to, for example, a maltose binding protein (“MBP”), glutathione S transferase (GST), hexahistidine, c-myc, or the FLAG epitope for ease of purification, monitoring expression, or monitoring cellular and subcellular localization.

Moreover, a nucleic acid encoding a fusion polypeptide of the present disclosure can be modified to improve expression of the protein in plants by using codon preference. When the nucleic acid is prepared or altered synthetically, advantage can be taken of known codon preferences of the intended plant host where the nucleic acid is to be expressed. For example, nucleic acids of the present disclosure can be modified to account for the specific codon preferences and GC content preferences of monocotyledons and dicotyledons, as these preferences have been shown to differ (Murray et al., Nucl. Acids Res. (1989) 17: 477-498).

The present disclosure further provides expression vectors encoding fusion polypeptides of the present disclosure. A nucleic acid sequence coding for the desired nucleic acid of the present disclosure can be used to construct an expression vector, which can be introduced into the desired host cell. An expression vector will typically contain a nucleic acid encoding a fusion polypeptide of the present disclosure, operably linked to transcriptional initiation regulatory sequences which will direct the transcription of the nucleic acid in the intended host cell, such as tissues of a transformed plant. Nucleic acids e.g. encoding fusion polypeptides of the present disclosure may be expressed on multiple expression vectors or they may be expressed on a single expression vector.

For example, plant expression vectors may include (1) a cloned gene under the transcriptional control of 5 and 3′ regulatory sequences and (2) a dominant selectable marker. Such plant expression vectors may also include, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

In some embodiments, expression of a nucleic acid of the present disclosure may be driven (in operable linkage) with a promoter (e.g. a promoter functional in plants or a plant-specific promoter). A plant promoter, or functional fragment thereof, can be employed to control the expression of a nucleic acid of the present disclosure in regenerated plants. The selection of the promoter used in expression vectors will determine the spatial and temporal expression pattern of the nucleic acid in the modified plant, e.g., the nucleic acid encoding the fusion polypeptide of the present disclosure is only expressed in the desired tissue or at a certain time in plant development or growth. Certain promoters will express nucleic acids in all plant tissues and are active under most environmental conditions and states of development or cell differentiation (i.e., constitutive promoters). Other promoters will express nucleic acids in specific cell types (such as leaf epidermal cells, mesophyll cells, root cortex cells) or in specific tissues or organs (roots, leaves or flowers; for example) and the selection will reflect the desired location of accumulation of the gene product. Alternatively, the selected promoter may drive expression of the nucleic acid under various inducing conditions.

Examples of suitable constitutive promoters may include, for example, the core promoter of the Rsyn7, the core CaMV 355 promoter (Odell et al., Nature (1985) 313:810-812), CaMV 19S (Lawton et al., 1987), rice actin (Wang et al., 1992; U.S. Pat. No. 5,641,876; and McElroy et al., Plant Cell (1985) 2:163-171); ubiquitin (Christensen et al., Plant Mol. Biol. (1989) 12:619-632; and Christensen et al., Plant Mol. Biol. (1992) 18:675-689), pEMU (Last et al., Theor. Appl. Genet. (1991) 81:581-588), MAS (Velton et al., EMBO J. (1984) 3:2723-2730), nos (Ebert et al., 1987), Adh (Walker et al.; 1987), the P- or 2′-promoter derived from T-DNA of Agrobacterium tumefaciens, the Smas promoter, the cinnamyl alcohol dehydrogenase promoter (U.S. Pat. No. 5,683,439), the Nos promoter, the pEmu promoter, the rubisco promoter, the GRP 1-8 promoter, and other transcription initiation regions from various plant genes known to those of skilled artisans, and constitutive promoters described in, for example, U.S. Pat. Nos. 5,608,149; 5,608,144; 5,604,121; 5,569,597; 5,466,785; 5,399,680; 5,268,463; and 5,608,142.

Examples of suitable tissue specific promoters may include, for example, the lectin promoter (Vodkin et al., 1983; Lindstrom et al., 1990), the corn alcohol dehydrogenase 1 promoter (Vogel et al., 1989; Dennis et al., 1984), the corn light harvesting complex promoter (Simpson, 1986; Bansal et al., 1992); the corn heat shock protein promoter (Odell et al., Nature (1985) 313:810-812; Rochester et al., 1986), the pea small subunit RuBP carboxylase promoter (Poulsen et al., 1986; Cashmore et al., 1983), the Ti plasmid mannopine synthase promoter (Langridge et al., 1989), the Ti plasmid nopaline synthase promoter (Langridge et al., 1989), the petunia chalcone isomerase promoter (Van Tunen et al., 1988), the bean glycine rich protein 1 promoter (Keller et al., 1989), the truncated CaMV 35s promoter (Odell et al., Nature (1985) 313:810-812), the potato patatin promoter (Wenzler et al., 1989), the root cell promoter (Conkling et al., 1990); the maize zein promoter (Reina et al., 1990; Kriz et al., 1987; Wandelt and Feix, 1989; Langridge and Feix, 1983; Reina et al., 1990), the globulin-1 promoter (Belanger and Kriz et al., 1991), the α-tubulin promoter, the cab promoter (Sullivan et al., 1989), the PEPCase promoter (Hudspeth & Grula, 1989), the R gene complex-associated promoters (Chandler et al., 1989), and the chalcone synthase promoters (Franken et al., 1991).

Alternatively, the plant promoter can direct expression of a nucleic acid of the present disclosure in a specific tissue or may be otherwise under more precise environmental or developmental control. Such promoters are referred to here as “inducible” promoters. Environmental conditions that may affect transcription by inducible promoters include, for example, pathogen attack, anaerobic conditions, or the presence of light. Examples of inducible promoters include, for example, the AdhI promoter which is inducible by hypoxia or cold stress; the Hsp70 promoter which is inducible by heat stress, and the PPDK promoter which is inducible by light. Examples of promoters under developmental control include, for example, promoters that initiate transcription only, or preferentially, in certain tissues, such as leaves, roots, fruit, seeds, or flowers. An exemplary promoter is the anther specific promoter 5126 (U.S. Pat. Nos. 5,689,049 and 5,689,051). The operation of a promoter may also vary depending on its location in the genome. Thus, an inducible promoter may become fully or partially constitutive in certain locations.

Moreover, any combination of a constitutive or inducible promoter, and a non-tissue specific or tissue specific promoter may be used to control the expression of various fusion polypeptides of the present disclosure.

The nucleic acids of the present disclosure and/or a vector housing a nucleic acid of the present disclosure, may also contain a regulatory sequence that serves as a 3′ terminator sequence. One of skill in the art would readily recognize a variety of terminators that may be used in the nucleic acids of the present disclosure. For example, a nucleic acid of the present disclosure may contain a 3′ NOS terminator.

In some embodiments, nucleic acids of the present disclosure contain a transcriptional termination site. Transcription termination sites may include, for example, OCS terminators and NOS terminators.

Plant transformation protocols as well as protocols for introducing nucleic acids of the present disclosure into plants may vary depending on the type of plant or plant cell, e.g., monocot or dicot, targeted for transformation. Suitable methods of introducing nucleic acids of the present disclosure into plant cells and optionally subsequent insertion into the plant genome include, for example, microinjection (Crossway et al, Biotechniques (1986) 4:320-334), electroporation (Riggs et al., Proc. Natl. Acad Sci. USA (1986) 83:5602-5606), Agrobacterium-mediated transformation (U.S. Pat. No. 5,563,055), direct gene transfer (Paszkowski et al., EMBO J. (1984) 3:2717-2722), and ballistic particle acceleration (U.S. Pat. No. 4,945,050; Tomes et al. (1995). “Direct DNA Transfer into Intact Plant Cells via Microprojectile Bombardment,” in Plant Cell, Tissue, and Organ Culture: Fundamental Methods; ed. Gamborg and Phillips (Springer-Verlag, Berlin); and McCabe et al., Biotechnology (1988) 6:923-926).

Additionally, fusion polypeptides of the present disclosure can be targeted to a specific organelle within a plant cell. Targeting can be achieved by providing the fusion protein with an appropriate targeting peptide sequence. Examples of such targeting peptides include, for example, secretory signal peptides (for secretion or cell wall or membrane targeting), plastid transit peptides, chloroplast transit peptides, mitochondrial target peptides, vacuole targeting peptides, nuclear targeting peptides, and the like (e.g., see Reiss et al., Mol. Gen. Genet. (1987) 209(1):116-121; Settles and Martienssen, Trends Cell Biol (1998) 12:494-501; Scott et al, J Biol Chem (2000) 10:1074; and Luque and Correas, J Cell Sci (2000) 113:2485-2495).

The modified plant may be grown into plants in accordance with conventional ways (e.g. McCormick et al., Plant Cell. Reports (1986) 81-84). These plants may then be grown, and pollinated with either the same transformed strain or different strains, with the resulting progeny having the desired phenotypic characteristic. Two or more generations may be grown to ensure that the subject phenotypic characteristic is stably maintained and inherited and then seeds harvested to ensure the desired phenotype or other property has been achieved.

The present disclosure also provides plants derived from plants having a genomic edit as a consequence of the methods of the present disclosure. A plant having a genomic edit as a consequence of the methods of the present disclosure may be crossed with itself or with another plant to produce an F1 plant. In some embodiments, one or more of the resulting F1 plants can also have a genomic edit of the target nucleic acid.

Further provided are methods of screening plants derived from plants having a genomic edit as a consequence of the methods of the present disclosure. In some embodiments, the derived plants (e.g. F1 or F2 plants resulting from or derived from crossing the plant having a genomic edit as a consequence of the methods of the present disclosure with another plant) can be selected from a population of derived plants. For example, provided are methods of selecting one or more of the derived plants that (i) lack the nucleic acid encoding the fusion polypeptide, and (ii) have a genomic edit of the target nucleic acid.

### EMBODIMENTS

The following numbered embodiments also form part of the present disclosure:
1. A fusion polypeptide comprising: (i) a cytidine deaminase domain comprising an amino acid sequence having at least 80%, at least 90%, at least 95%, at least 98%, or at least 99% sequence identity to the amino acid sequence set forth in SEQ ID NO: 2, 6, 10, 14, 16, 17, 31, 37-40, 43-51, 53, 54, 56, 58-61, 63, or 64; and (ii) a DNA binding domain, optionally wherein the DNA binding domain is an RNA-guided DNA binding domain.
2. The fusion polypeptide of embodiment 1, wherein the cytidine deaminase domain comprises an amino acid sequence having at least 80%, at least 90%, at least 95%, at least 98%, or at least 99% sequence identity to the amino acid sequence set forth in SEQ ID NO: 2, 6, 14, 16, 31, 37, 38, 43, 45, 51, 53, 56, 59-61, or 63.
3. The fusion polypeptide of embodiment 1 or embodiment 2, wherein the cytidine deaminase domain comprises an amino acid sequence having at least 80%, at least 90%, at least 95%, at least 98%, or at least 99% sequence identity to the amino acid sequence of SEQ ID NO: 6, 16, 37, 38, 45, or 53.
4. The fusion polypeptide of any one of embodiments 1-3, wherein the cytidine deaminase domain comprises the amino acid sequence of SEQ ID NO: 6, 16, 37, 38, 45, or 53.
5. The fusion polypeptide of any one of embodiments 1-4, wherein the DNA binding domain comprises a Cas9 domain, a Cas12a domain, a Cas12b domain, a zinc finger domain, or a transcription activator-like effector (TALE) domain.
6. The fusion polypeptide of any one of embodiments 1-5, wherein the RNA-guided DNA binding domain is nuclease active, nuclease inactive, or a nickase.
7. The fusion polypeptide of any one of embodiments 1-6, wherein the RNA-guided DNA binding domain comprises an amino acid sequence having at least 80%, at least 90%, at least 95%, at least 98%, or at least 99% sequence identity to the amino acid sequence of SEQ ID NO: 133.
8. The fusion polypeptide of any one of embodiments 1-7, further comprising a uracil glycosylase inhibitor (UGI) domain.
9. The fusion polypeptide of embodiment 8, wherein the UGI comprises an amino acid sequence having at least 80%, at least 90%, at least 95%, at least 98%, or at least 99% sequence identity to the amino acid sequence of SEQ ID NO: 135.
10. The fusion polypeptide of any one of embodiments 1-9, further comprising a nuclear localization signal (NLS).
11. The fusion polypeptide of any one of embodiments 1-10, wherein the fusion polypeptide comprises the structure: NH2-[cytidine deaminase domain]-[first NLS]-[RNA-guided DNA binding domain]-[second NLS]-[UGI]-[third NLS]-COOH, and wherein each instance of “-” optionally comprises a linker.
12. A complex comprising the fusion polypeptide of any one of embodiments 1-11 and a DNA-targeting RNA bound to the RNA-guided DNA binding domain of the fusion polypeptide.
13. A cell comprising the fusion polypeptide of any one of embodiments 1-11 or the complex of embodiment 12.
14. The cell of embodiment 13, wherein the cell is a plant cell.
15. A polynucleotide encoding the fusion polypeptide of any one of embodiments 1-11.
16. The polynucleotide of embodiment 16, wherein the cytidine deaminase domain comprises a nucleotide sequence having at least 80%, at least 90%, at least 95%, at least 98%, or at least 99% sequence identity to the nucleotide sequence set forth in SEQ ID NO: 68, 72, 76, 80, 82, 83, 97, 103-106, 109-117, 119, 120, 122, 124-127, 129, or 130.
17. The polynucleotide of embodiment 15 or embodiment 16, wherein the polynucleotide encoding the fusion polypeptide is codon-optimized for expression in a plant cell.
18. A vector comprising the polynucleotide of any one of embodiments 15-17.
19. The vector of embodiment 18, wherein the vector comprises a heterologous promotor driving expression of the polynucleotide.
20. A cell comprising the polynucleotide of any one of embodiments 15-17 or the vector of embodiment 18 or 19, optionally wherein the cell is a plant cell.
21. A method of modifying a target nucleic acid, the method comprising: contacting the target nucleic acid with: (a) a fusion polypeptide comprising: (i) a cytidine deaminase domain comprising an amino acid sequence having at least 80%, at least 90%, at least 95%, at least 98%, or at least 99% sequence identity to the amino acid sequence set forth in SEQ ID NO: 2, 6, 10, 14, 16, 17, 31, 37-40, 43-51, 53, 54, 56, 58-61, 63, or 64, and (ii) a DNA binding domain, optionally wherein the DNA binding domain is an RNA-guided DNA binding domain; and (b) optionally a DNA-targeting RNA, wherein the DNA-targeting RNA is capable of forming a complex with the RNA-guided DNA binding domain of the fusion polypeptide and directing the complex to the target nucleic acid, resulting in one or more C to T substitutions.
22. The method of embodiment 21, wherein the cytidine deaminase domain comprises an amino acid sequence having at least 80%, at least 90%, at least 95%, at least 98%, or at least 99% sequence identity to the amino acid sequence set forth in SEQ ID NO: 2, 6, 14, 16, 31, 37, 38, 43, 45, 51, 53, 56, 59-61, or 63.
23. The method of embodiment 21 or embodiment 22, wherein the cytidine deaminase domain comprises an amino acid sequence having at least 80%, at least 90%, at least 95%, at least 98%, or at least 99% sequence identity to the amino acid sequence of SEQ ID NO: 6, 16, 37, 38, 45, or 53.
24. The method of any one of embodiments 21-23, wherein the cytidine deaminase domain comprises the amino acid sequence of SEQ ID NO: 6, 16, 37, 38, 45, or 53.
25. The method of any one of embodiments 21-24, wherein the DNA binding domain comprises a Cas9 domain, a Cas12a domain, a Cas12b domain, a zinc finger domain, or a transcription activator-like effector (TALE) domain.
26. The method of any one of embodiments 21-25, wherein the RNA-guided DNA binding domain is nuclease active, nuclease inactive, or a nickase.
27. The method of any one of embodiments 21-26, wherein the RNA-guided DNA binding domain comprises an amino acid sequence having at least 80%, at least 90%, at least 95%, at least 98%, or at least 99% sequence identity to the amino acid sequence of SEQ ID NO: 133.
28. The method of any one of embodiments 21-27, wherein the fusion polypeptide further comprises a uracil glycosylase inhibitor (UGI) domain.
29. The method of embodiment 28, wherein the UGI comprises an amino acid sequence having at least 80%, at least 90%, at least 95%, at least 98%, or at least 99% sequence identity to the amino acid sequence of SEQ ID NO: 135.
30. The method of any one of embodiments 21-29, further comprising a nuclear localization signal (NLS).
31. The method of any one of embodiments 21-30, wherein the fusion polypeptide comprises the structure: NH2-[cytidine deaminase domain]-[first NLS]-[RNA-guided DNA binding domain]-[second NLS]-[UGI]-[third NLS]-COOH, and wherein each instance of “-” optionally comprises a linker.
32. The method of any one of embodiments 21-30, wherein the target nucleic acid is in a cell.
33. The method of embodiment 32, wherein the cell is a plant cell.
34. The method of any one of embodiments 21-33, wherein the contacting is at a temperature from about 22° C. to about 32° C. or about 22° C. to about 25° C., optionally wherein the temperature is about 22° C. or about 25° C.
35. A method for producing a genetically modified plant, the method comprising: introducing into the plant: (a) the fusion polypeptide of any one of embodiments 1-11, or a polynucleotide encoding the fusion polypeptide; and (b) optionally a DNA-targeting RNA, or a DNA polynucleotide encoding the DNA-targeting RNA, wherein the DNA-targeting RNA is capable of forming a complex with the RNA-guided DNA binding domain of the fusion polypeptide and directing the complex to a target nucleic acid in the genome of the plant, resulting in one or more C to T substitutions.
36. The method of embodiment 35, wherein the introducing is at a temperature from about 22° C. to about 32° C. or about 22° C. to about 25° C., optionally wherein the temperature is about 22° C. or about 25° C.
37. The method of embodiment 35 or embodiment 36, wherein the plant is a monocotyledonous or a dicotyledonous species.
38. The method of any one of embodiments 35-37, wherein the plant is Oryza sativa or Solanum lycopersicum.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, it will be obvious that certain changes and modifications may be practiced within the scope of the appended claims.

The following examples are offered by way of illustration and not by way of limitation.

### EXAMPLES

### Example 1: Identification and Selection of Novel Cytidine Deaminases

Cytidine deaminase candidates were identified using the protein sequences for the rAPOBEC1, PmCDA1, hA3A, hAID, MwA3G, and NbaA3X2 ORFs as queries in the National Center of Biotechnology Information (NCBI) RefSeq database using the protein Basic Local Alignment Search Tool (BLASTp). NCBI RefSeq database was used to identify prospective cytidine deaminases because it contains the most recently sequenced, annotated, and diverse ORFs which are publicly available.

Candidates were selected based upon optimal species temperature as determined by the publicly available literature with an emphasis on NCBI catalogued published sources and an expected protein sequence length of 150-300 amino acids. All selected cytidine deaminase ORFs were determined to be <80% identical to the query sequences at the time of the search and initiation of testing.

### Example 2: Screening of Cytidine Deaminases in Rice Protoplasts

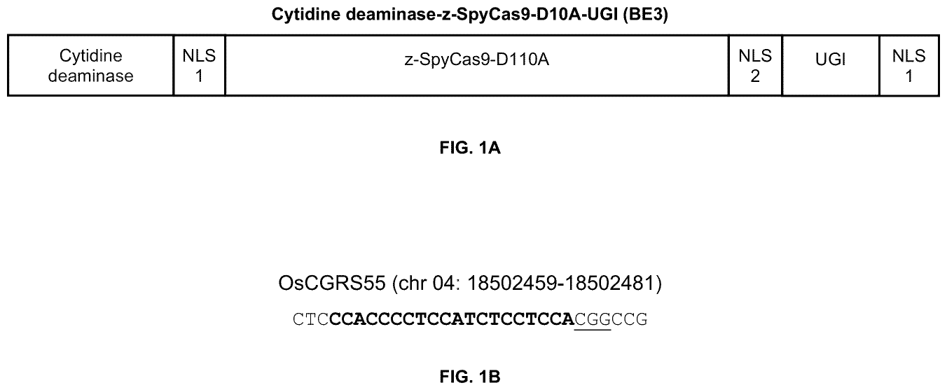

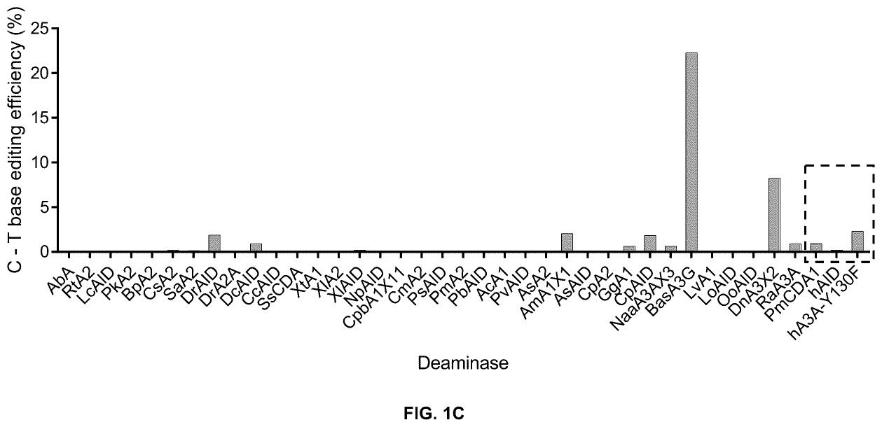

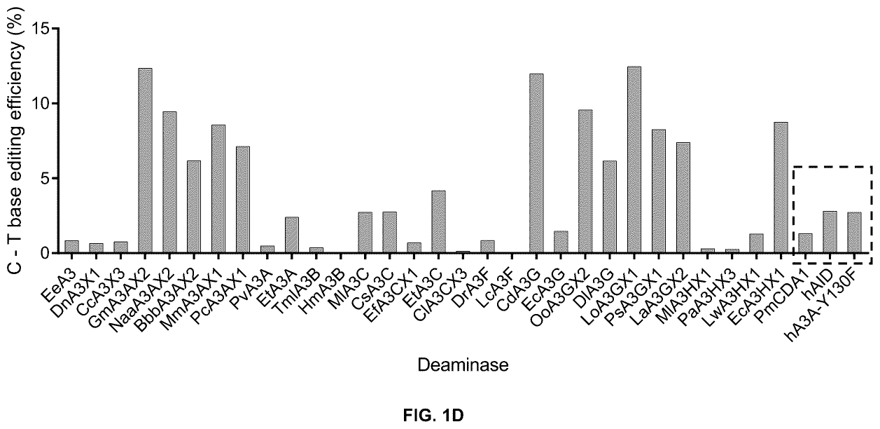

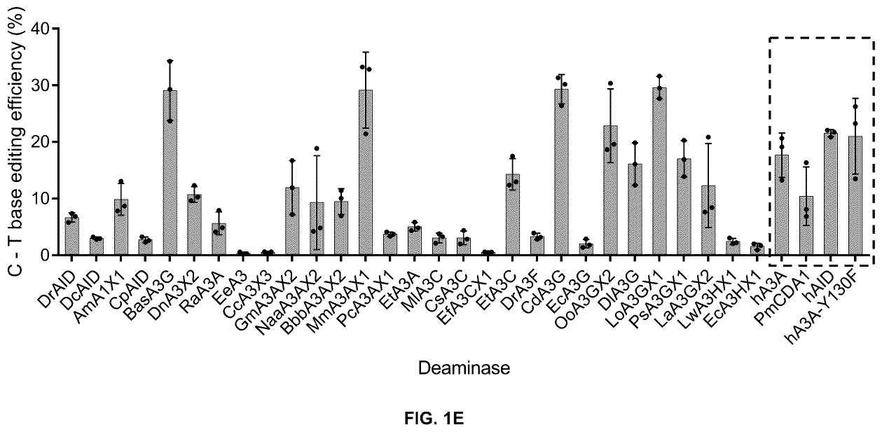

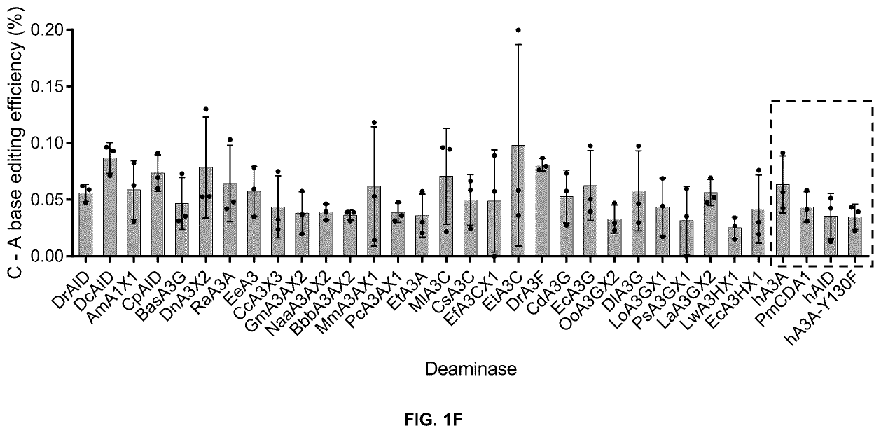

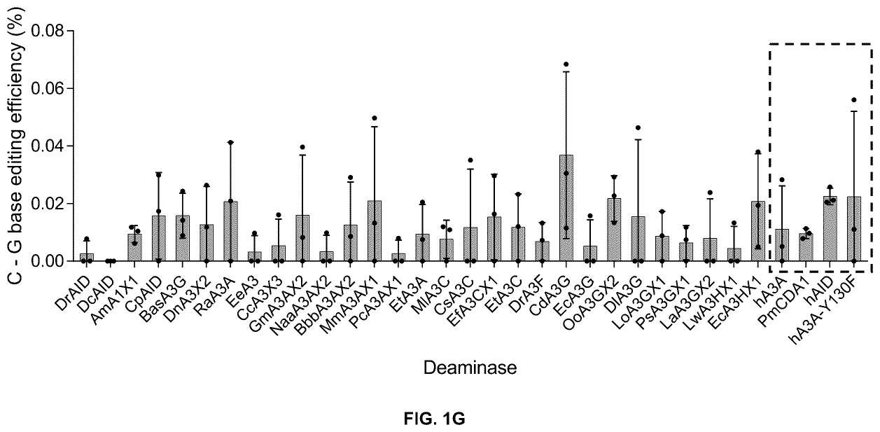

The novel cytidine deaminases were coupled to BE3 architecture (FIG. 1A) and the final expression vectors (DNA reagents) were transformed into freshly isolated rice protoplasts using PEG mediated transformation. Three broadly used cytidine deaminases for base editing in plants were used as controls and for benchmarking, namely PmCDA1, hAID, and hA3A-Y130F. Base editing efficiency was determined using next generation sequencing (NGS) of PCR amplicons of the target site within OsCGRS55 gene (FIG. 1B). A first batch of 36 candidates was selected and tested for efficiency based upon taxonomic distribution and sequence alignment with the queried sequence, as determined by Clustal Omega. The protein sequences of the cytidine deaminase ORFs tested in the first batch are listed in Table 1. Preliminary results of C-to-T base editing efficiency in rice protoplasts with cytidine deaminases of the first batch are presented in FIG. 1C. For preliminary testing, three biological replicates were pooled together.

A second batch of 30 additional cytidine deaminases were tested using rice protoplast assay and NGS based validation of base editing comprised of specific APOBEC3 families based upon the C-to-T conversion efficiency of the first batch cytidine deaminases, using the Minke Whale and Nine-banded armadillo sequences identified in the first round of candidates as baits in the NCBI RefSeq database. The protein sequences for the cytidine deaminase ORFs tested in second batch are listed in Table 2. The results of C to T base editing efficiency in rice protoplasts with cytidine deaminases of second batch are presented in FIG. 1D. For preliminary testing, three biological replicates were pooled together.

The results of the first and second batches showed that multiple novel cytidine deaminases, when incorporated into a Cas9-based cytosine base editor (BE3) system, outperformed the existing technology represented by three main CBEs (PmCDA1, hAID, hA3A/130F).

### Example 3: Base Editing in Rice Protoplasts

Based on the preliminary screening, 29 novel CBEs (SEQ ID NOs: 2, 6, 10, 14, 16, 17, 31, 37-40, 43-51, 53, 54, 56, 58-61, 63, and 64) were chosen to be further evaluated at the same target site (OsCGRS55) in rice. The results of C-to-T testing with 3 biological replicates are depicted in FIG. 1E. Additionally, a fourth cytidine deaminase (hA3A) CBE was used as an existing technology alongside the previously used PmCDA1, hAID, hA3A/130F based CBEs. C-to-A and C-to-G conversion at the same target site were also evaluated (FIG. 1F-G). The results demonstrate that 14 novel deaminases identified can achieve similar or superior editing efficiency than the existing technologies represented by hA3A, PmCDA1, hAID, and hA3A/Y130F CBE. The C-to-A and C-to-G conversions were very low and comparable to the established CBEs, indicating high levels of C-to-T base editing purity.

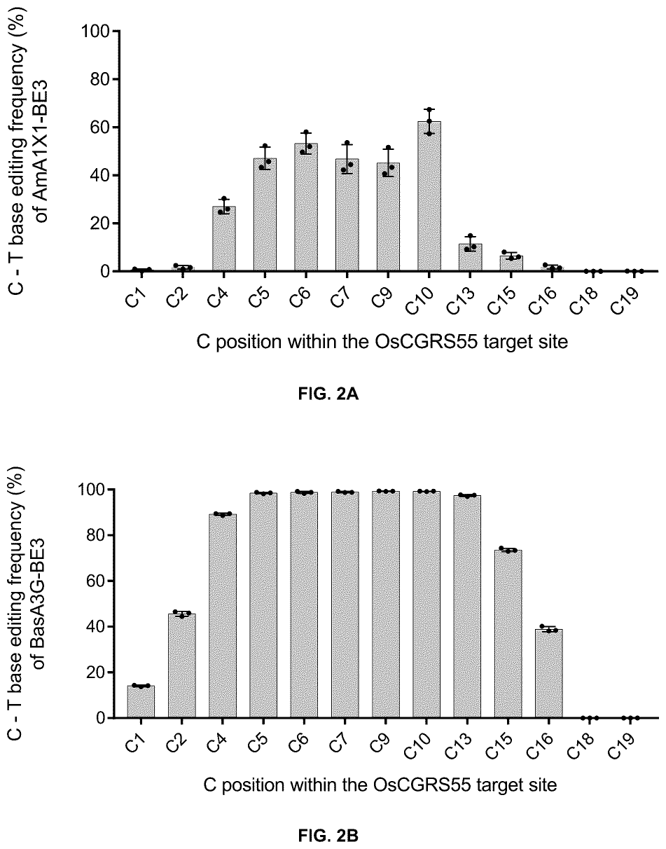

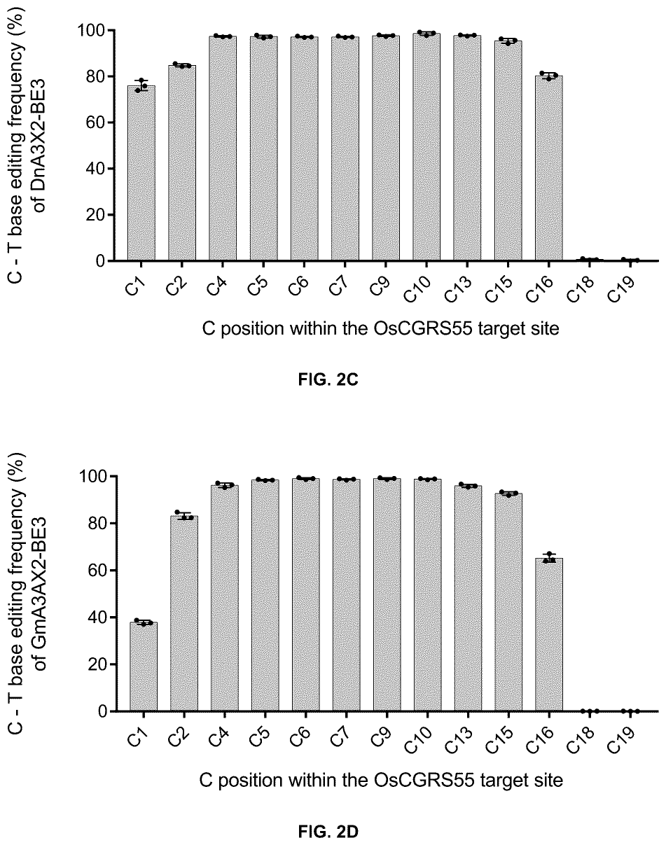

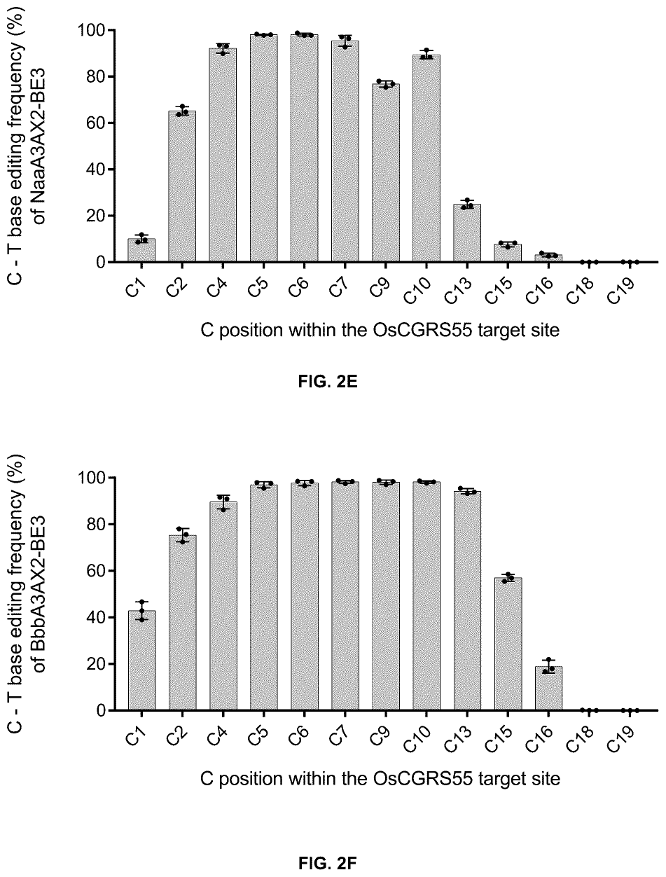

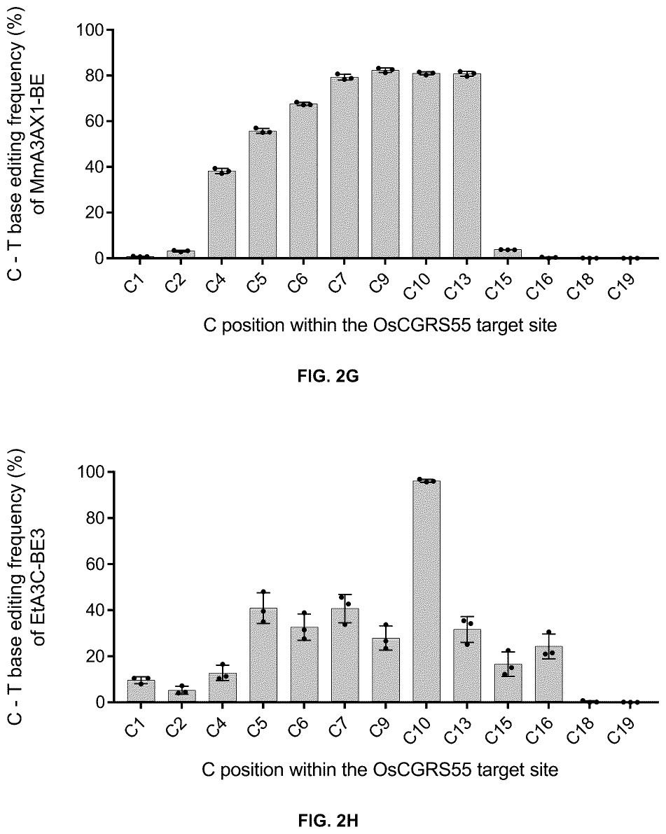

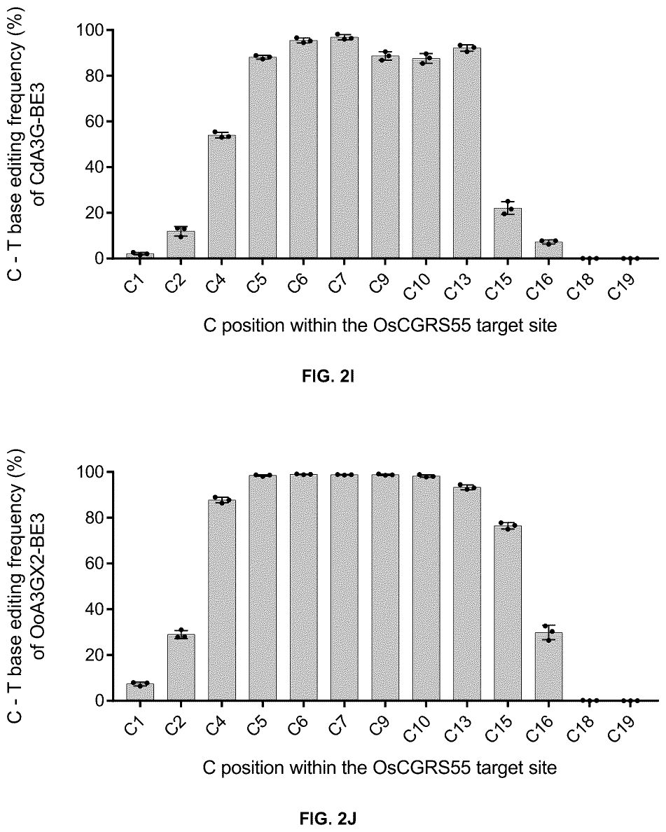

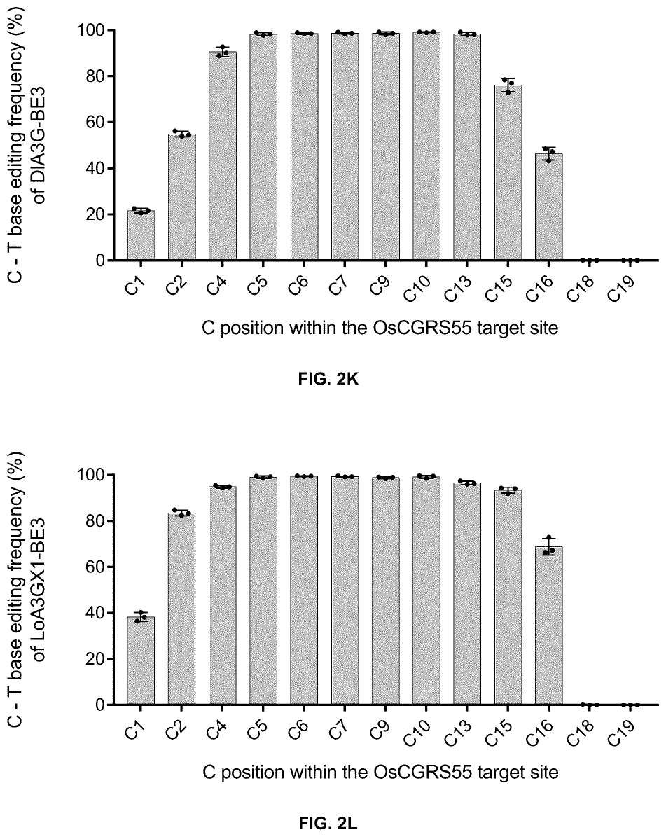

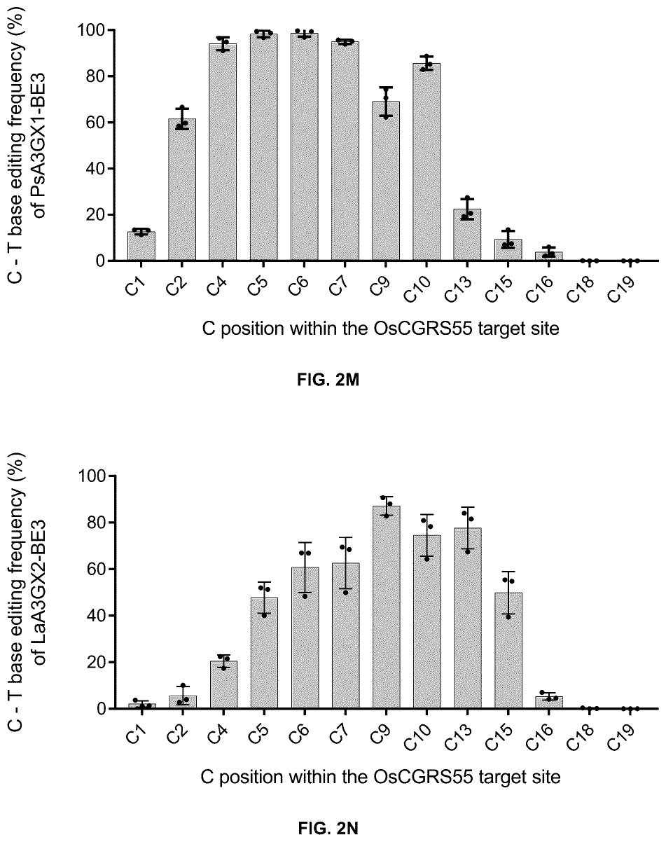

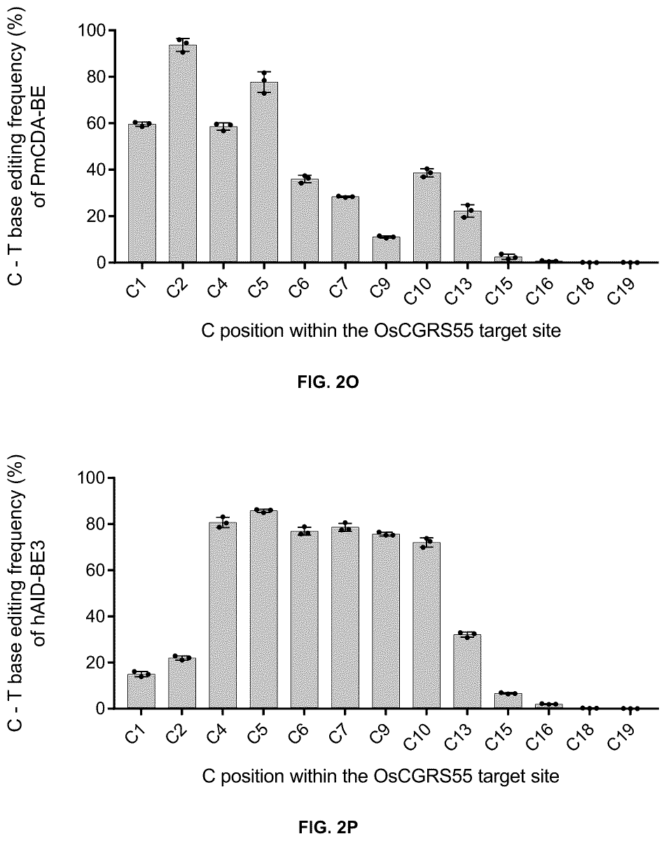

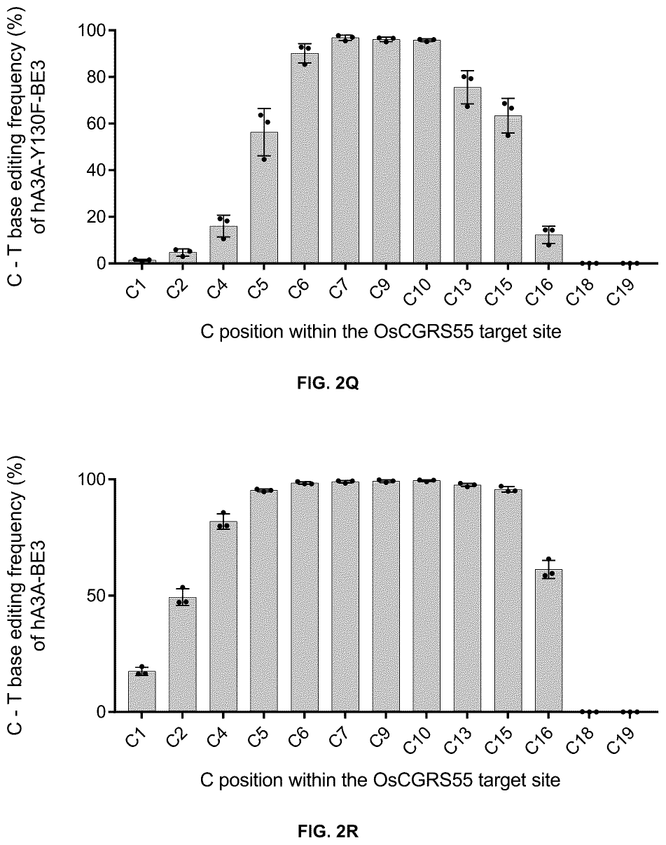

Different base editing activity windows at the same target site were revealed for 14 novel CBEs (FIG. 2). EtA3C-CBE demonstrated a very narrow activity window (e.g., around the C10 position) compared to other tested deaminases with broad activity window spanning from C4 to C13 positions (FIG. 211). Such CBEs with narrow editing windows are useful for direct specific base changes without introducing byproduct edits. Surprisingly, DnA3X2-CBE had activity window spanning from C1 to C16 with each tested cytosine position within the target site exhibiting more than 70% C-to-T conversion frequency, significantly outperforming the established technologies (FIG. 2C). Such CBEs with broad editing windows are powerful tools for directed evolution of a target gene due to the broad coverage of target bases.

The 29 deaminases were tested at another C-rich target site within OsCGRS57 gene in rice protoplasts. The base editing was verified by Sanger sequencing at the OsCGRS57 target site, and the base editing windows were assessed. DnA3X2-CBE demonstrated wide editing window at OsCGRS57 target site spanning from C3 to C16, very similar to editing window at OsCGRS55. Conversely, EtA3C-CBE demonstrated a narrow editing window at OsCGRS55 target site and a rather wide editing window at OsCGRS57 target site spanning from C4 to C16. Interestingly, editing stretching upstream of 5′ end of OsCGRS57 target site was observed in the case of LwA3HX1-CBE. A broad editing window, such as LwA3HX1-CBE, can be used to edit cytosines with base editors coupled with canonical SpCas9 when there are limited choices of suitable PAMs in their vicinity.

### Example 4: Base Editing in Tomato Protoplasts

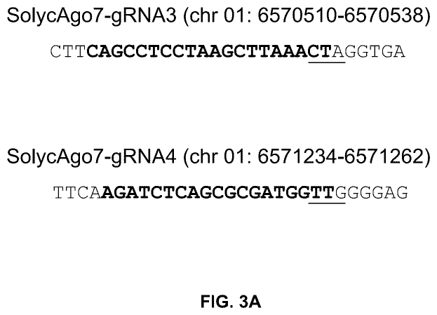

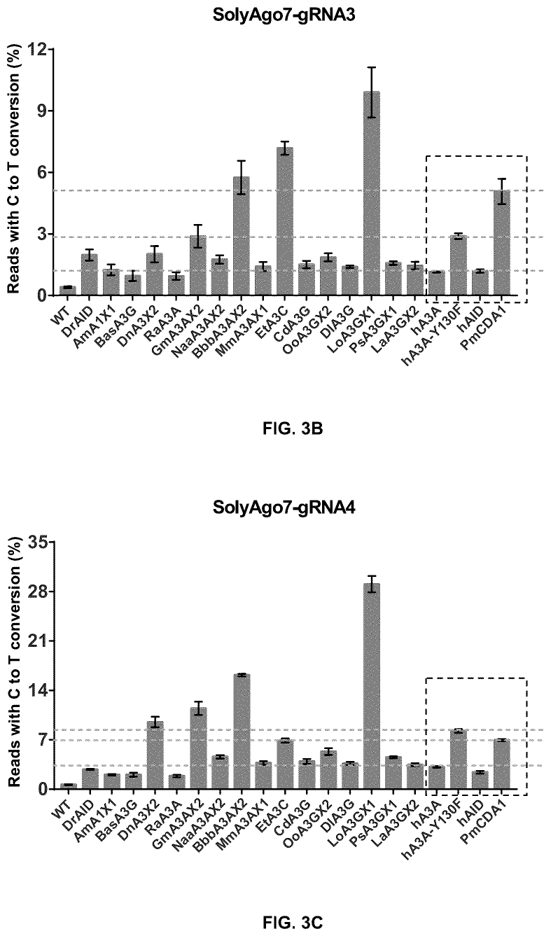

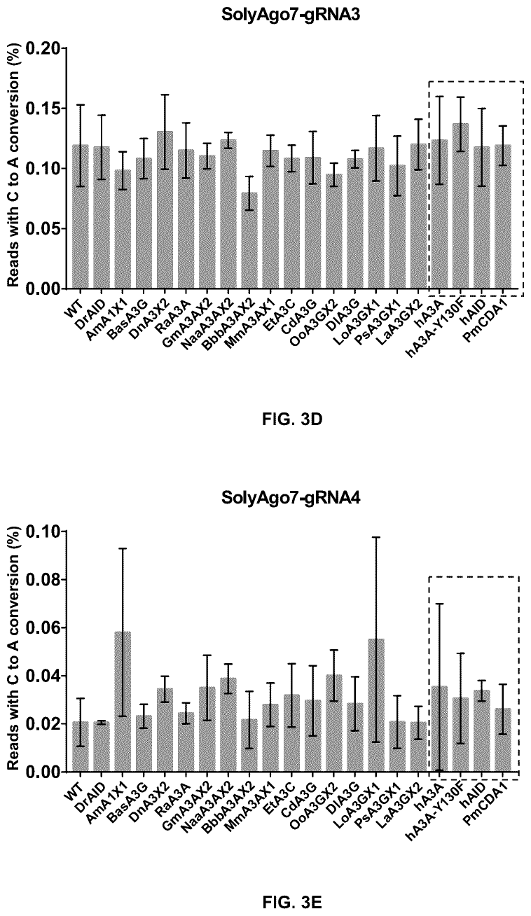

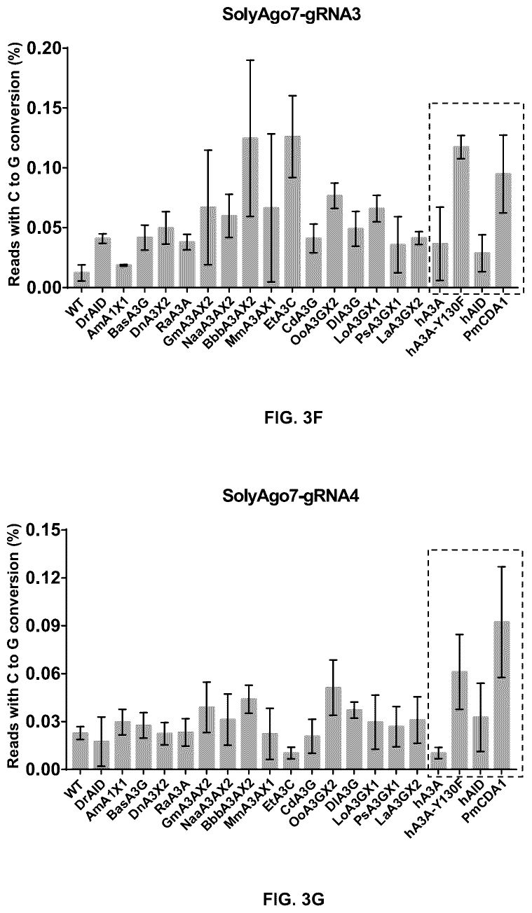

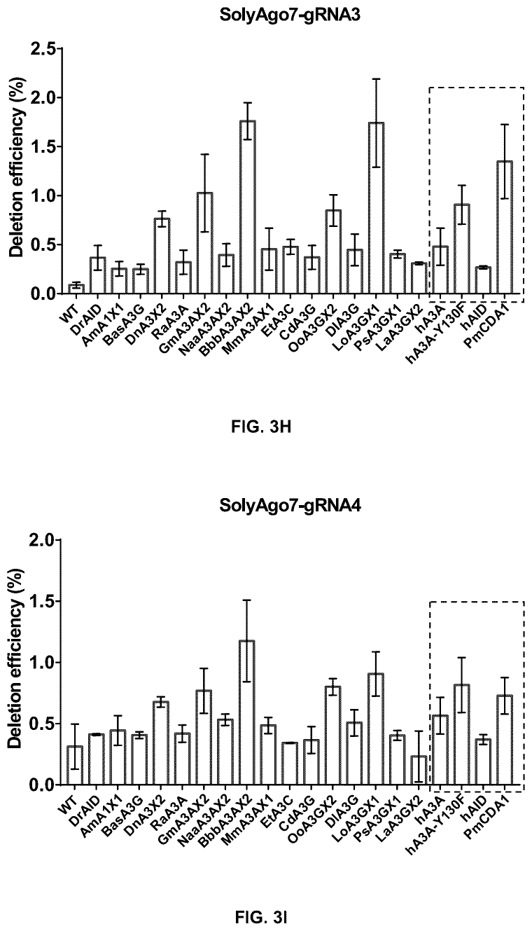

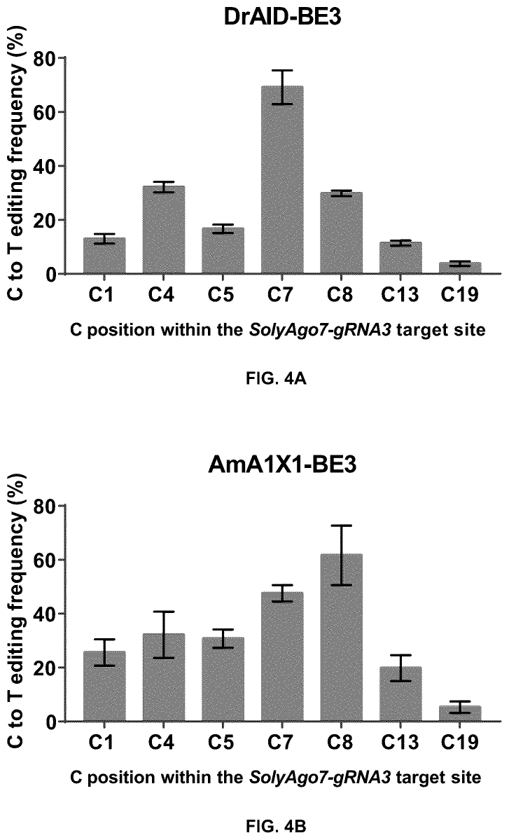

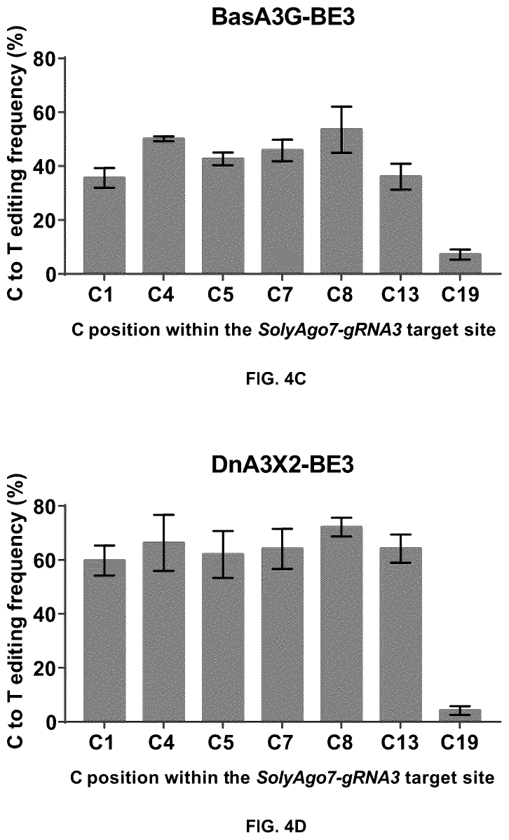

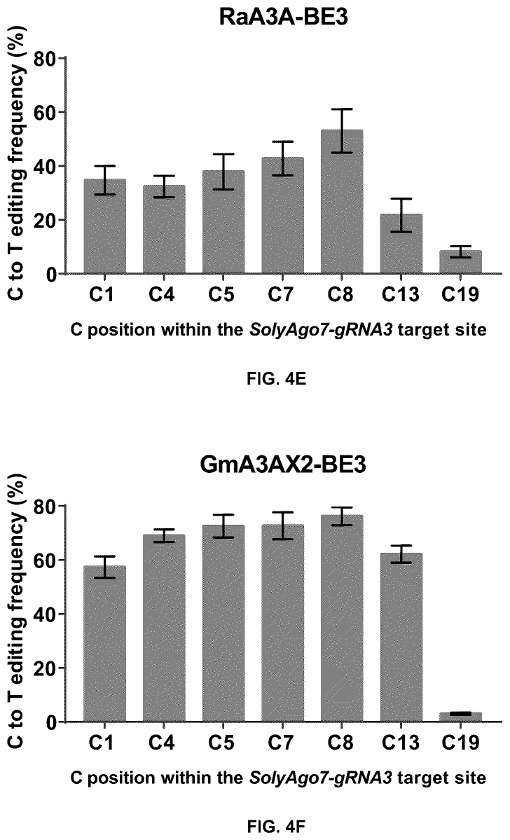

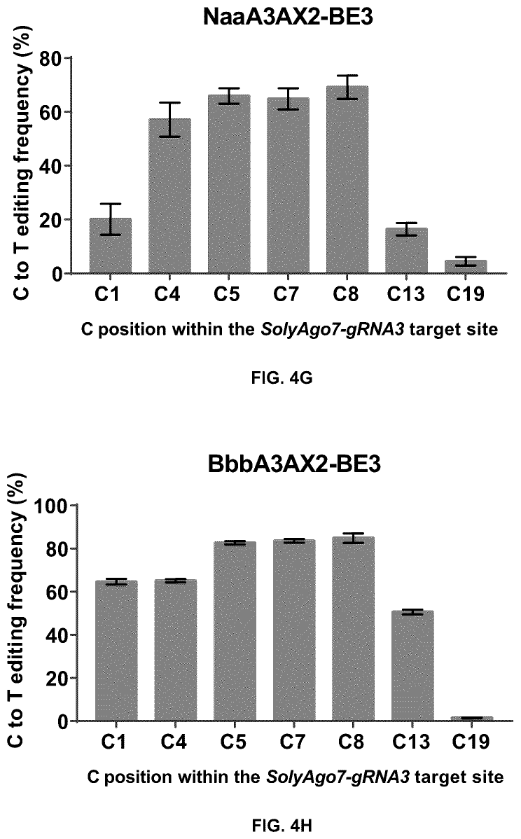

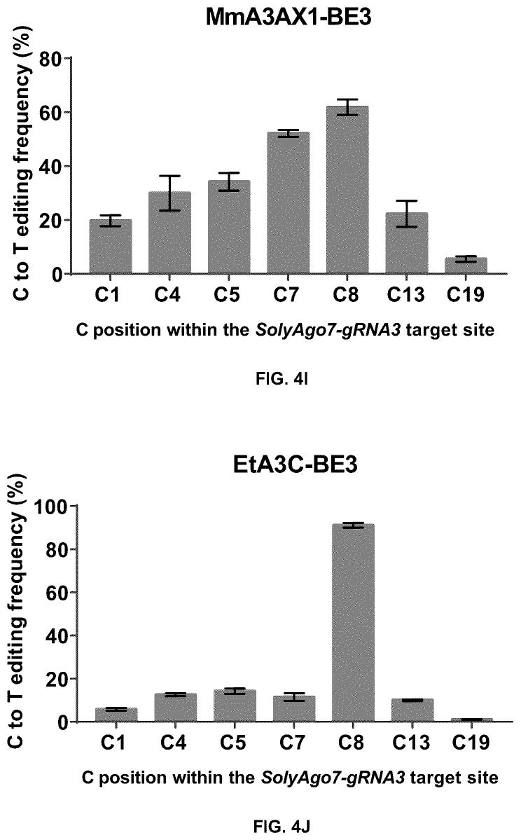

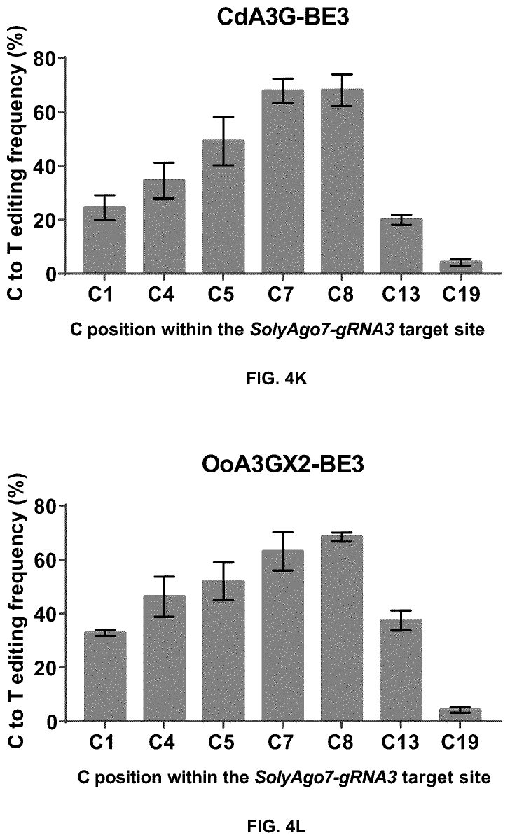

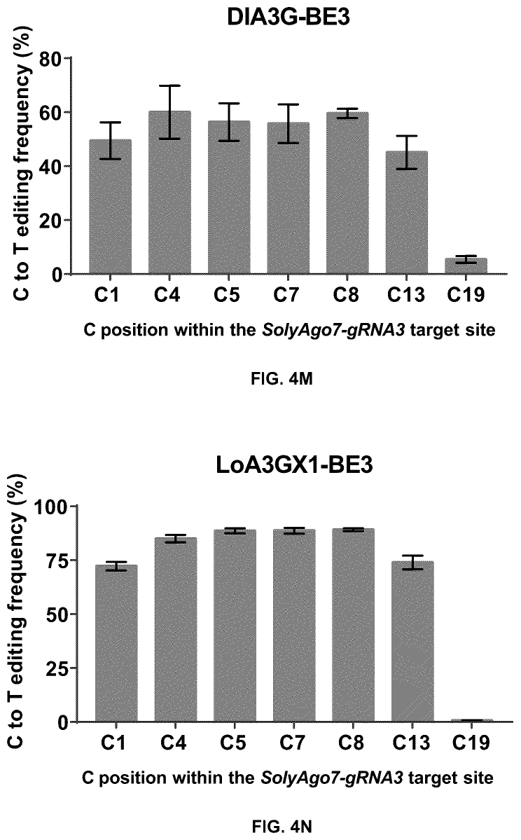

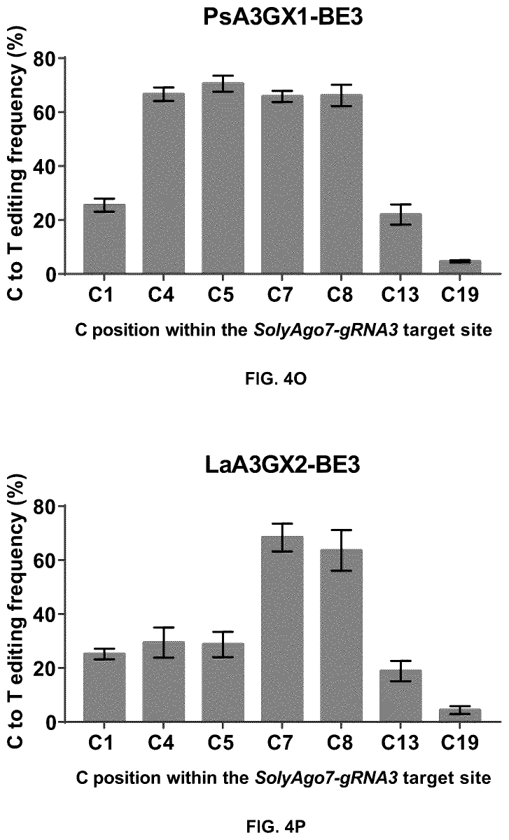

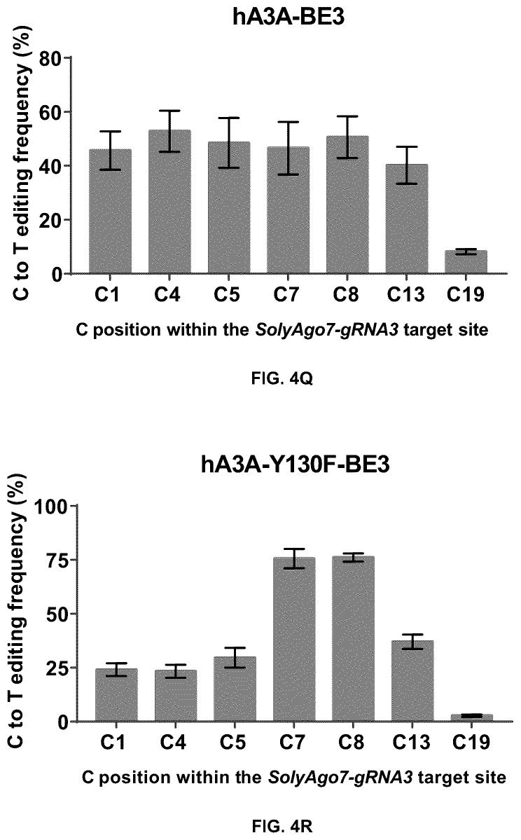

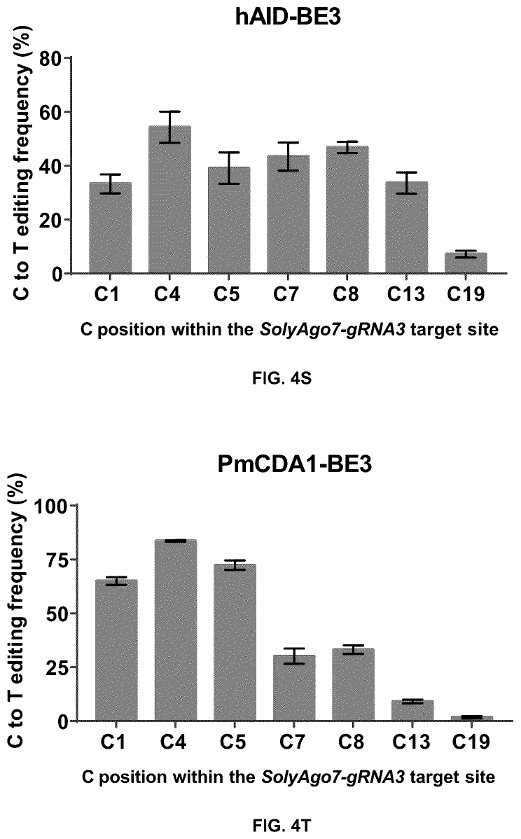

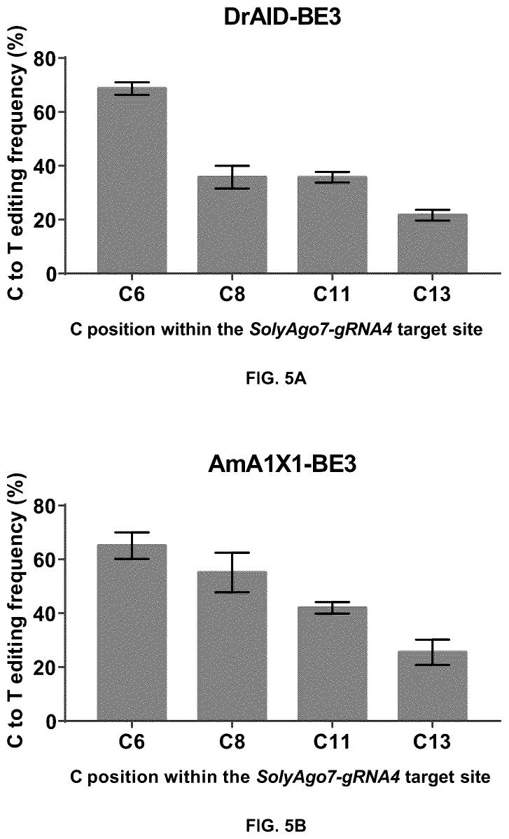

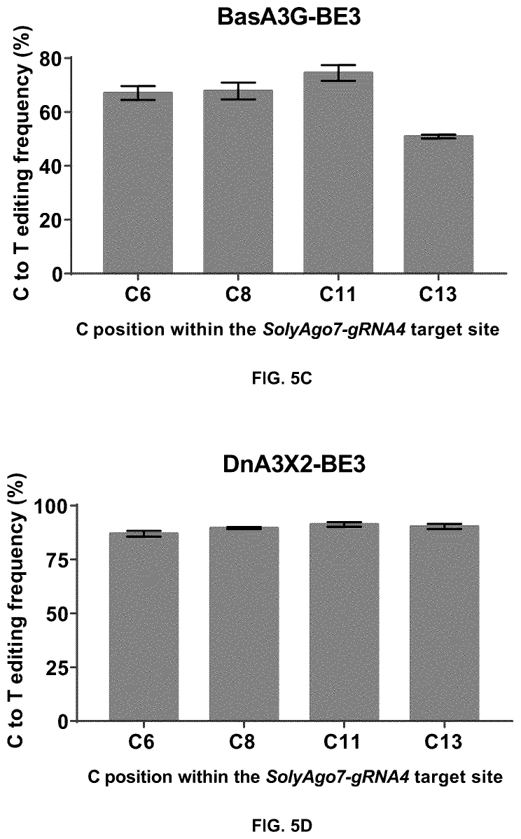

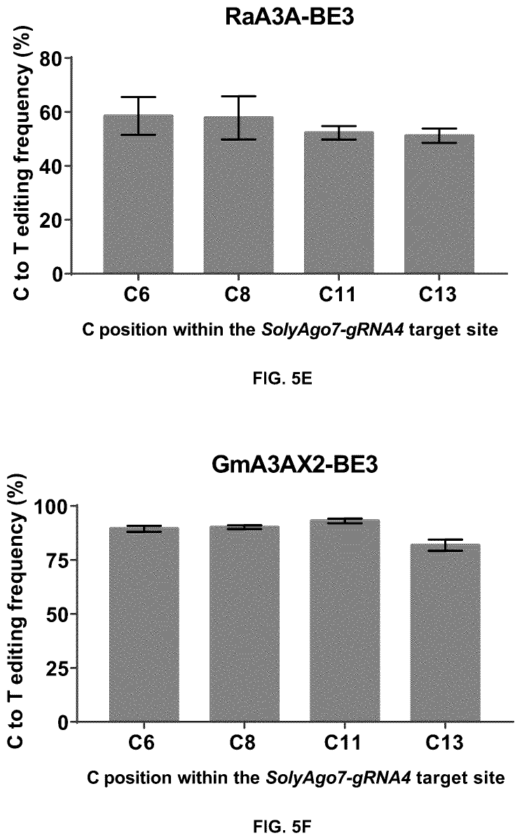

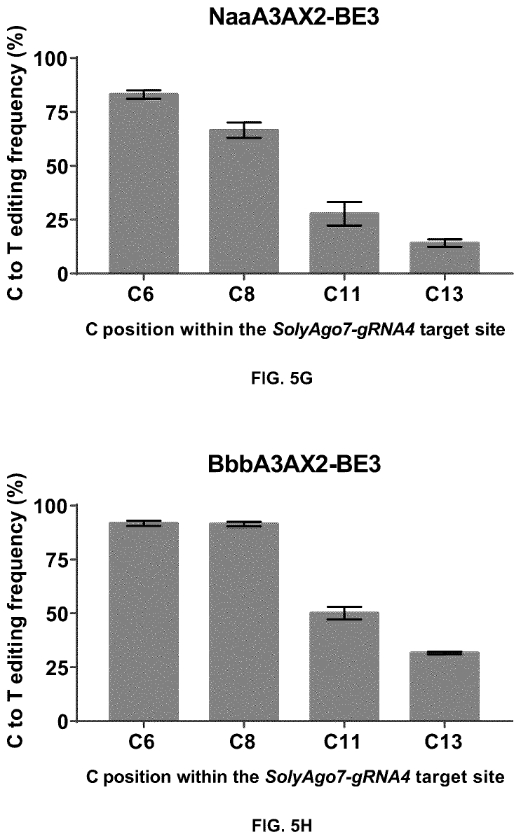

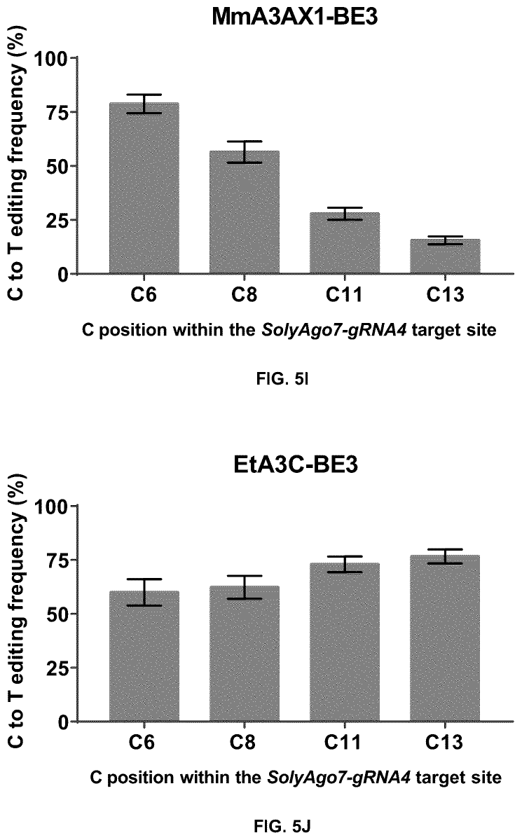

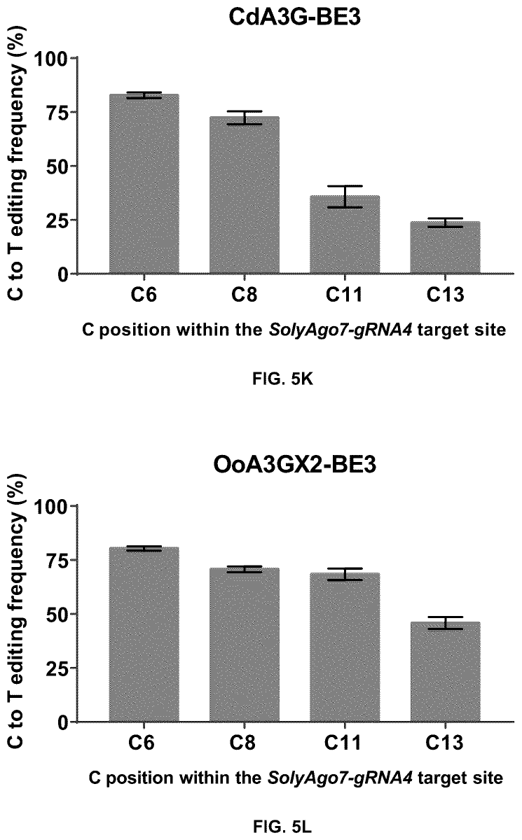

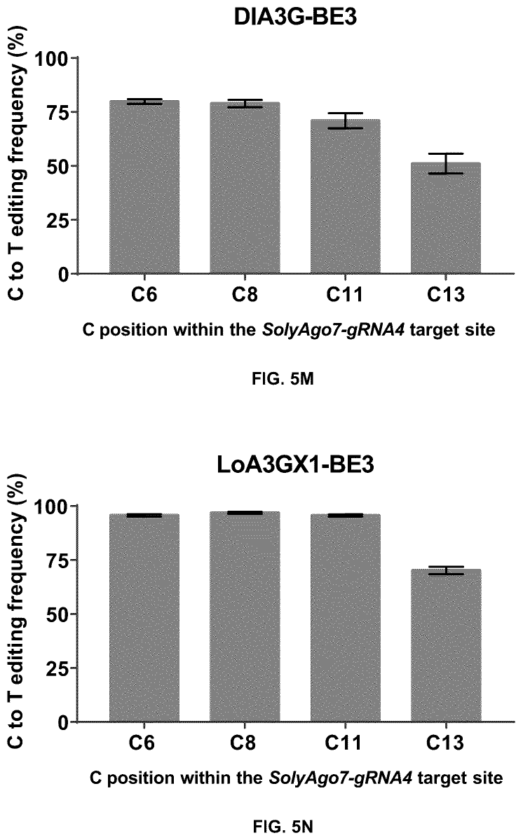

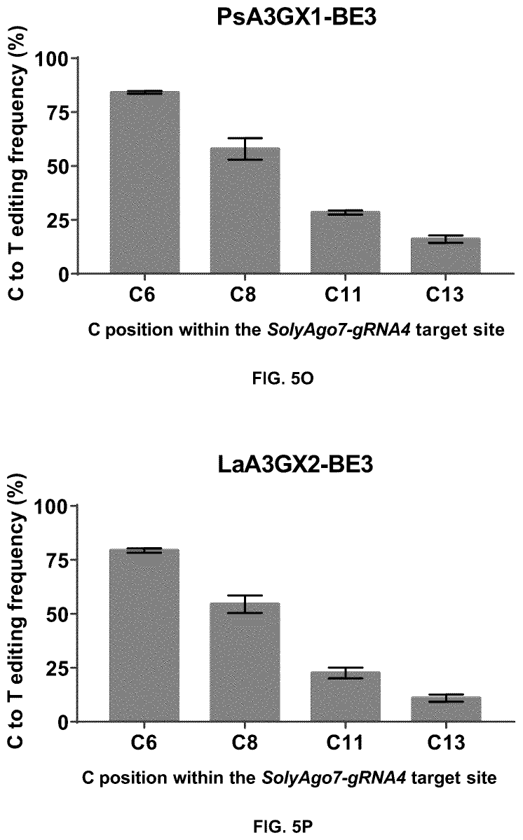

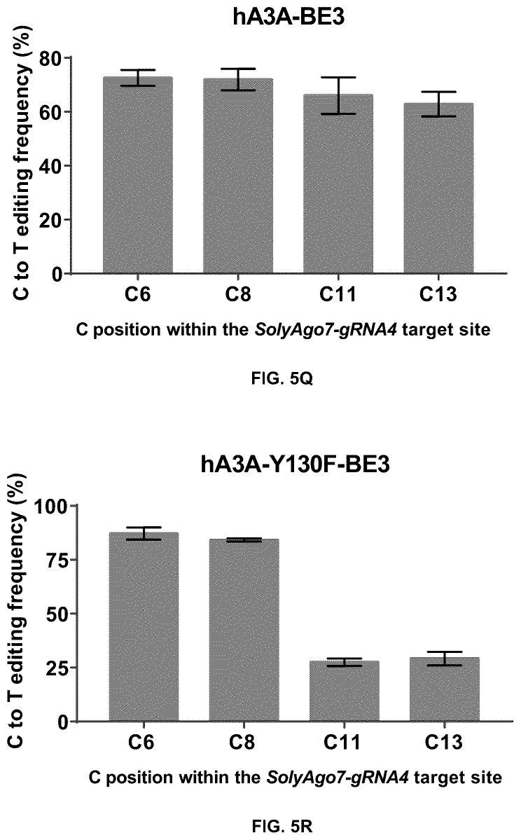

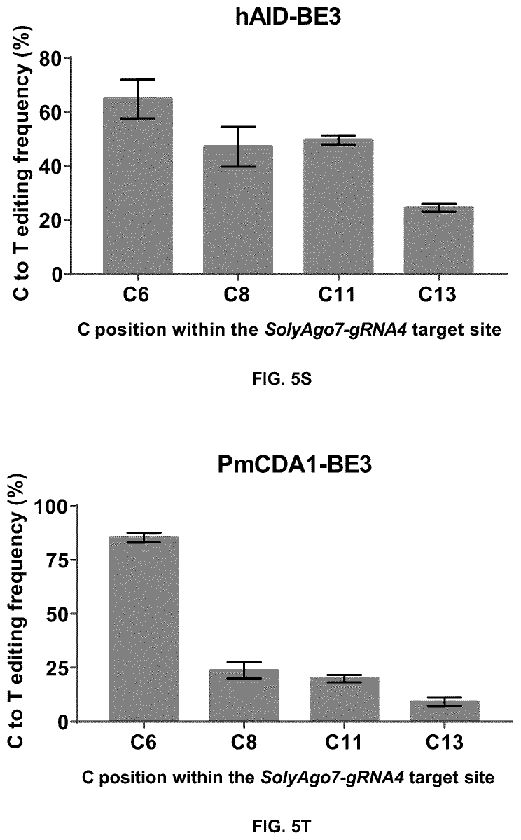

Sixteen best performing CBEs (SEQ ID NOs: 2, 6, 14, 16, 31, 37, 38, 43, 45, 51, 53, 56, 59-61, and 63) were selected for further assessment in tomato protoplasts at two independent target sites in the AGO7 (SolyA7) gene (FIG. 3A). Sixteen CBEs at SolyAgo7-gRNA3 (FIG. 3B) and SolyAgo7-gRNA4 (FIG. 3C) target sites performed at least as well as the existing CBEs represented by hA3A, PmCDA1, hAID, and hA3A/Y130F CBE, while three outperformed the existing CBEs. At SolyAgo7-gRNA4 target site, the overall C-T editing efficiency was higher and the difference between top performing novel CBE (with Loa3GX1 deaminase) compared to SolyAgo7-gRNA3 target site was higher as well. The C-to-A and C-to-G conversions with the novel CBEs were comparable to the established CBEs and barely above the background mutation rates, indicating high base editing purity (FIG. 3D-G). Similarly, low frequencies of indel byproducts were observed among the editing outcomes of these novel CBEs (FIG. 311, FIG. 3I). Base editing windows of novel CBEs in tomato (FIG. 4, FIG. 5) showed high similarity to the observation in rice with EtA3C-CBE demonstrating a very narrow activity window focused on C8 at SolyAgo7-gRNA3 target site (FIG. 4J) but not at SolyAgo7-gRNA4 target site (FIG. 5J). DnA3X2-CBE demonstrated a broad activity window at both tested target sites (FIG. 4D, FIG. 5D).

What is claimed is:

1. A fusion polypeptide comprising: (i) a cytidine deaminase domain comprising an amino acid sequence having at least 80%, at least 90%, at least 95%, at least 98%, or at least 99% sequence identity to the amino acid sequence set forth in SEQ ID NO: 2, 6, 10, 14, 16, 17, 31, 37-40, 43-51, 53, 54, 56, 58-61, 63, or 64; and (ii) an RNA-guided DNA binding domain.

2. The fusion polypeptide of claim 1, wherein the cytidine deaminase domain comprises an amino acid sequence having at least 80%, at least 90%, at least 95%, at least 98%, or at least 99% sequence identity to the amino acid sequence set forth in SEQ ID NO: 2, 6, 14, 16, 31, 37, 38, 43, 45, 51, 53, 56, 59-61, or 63.

3. The fusion polypeptide of claim 1, wherein the cytidine deaminase domain comprises an amino acid sequence having at least 80%, at least 90%, at least 95%, at least 98%, or at least 99% sequence identity to the amino acid sequence of SEQ ID NO: 6, 16, 37, 38, 45, or 53.

4. The fusion polypeptide of claim 1, wherein the cytidine deaminase domain comprises the amino acid sequence of SEQ ID NO: 6, 16, 37, 38, 45, or 53.

5. The fusion polypeptide of claim 1, wherein the RNA-guided DNA binding domain comprises a Cas9 domain, a Cas12a domain, or a Cas12b domain.

6. The fusion polypeptide of claim 1, wherein the RNA-guided DNA binding domain is nuclease active, nuclease inactive, or a nickase.

7. The fusion polypeptide of claim 1, wherein the RNA-guided DNA binding domain comprises an amino acid sequence having at least 80%, at least 90%, at least 95%, at least 98%, or at least 99% sequence identity to the amino acid sequence of SEQ ID NO: 133.

8. The fusion polypeptide of claim 1, further comprising a uracil glycosylase inhibitor (UGI) domain.

9. The fusion polypeptide of claim 8, wherein the UGI comprises an amino acid sequence having at least 80%, at least 90%, at least 95%, at least 98%, or at least 99% sequence identity to the amino acid sequence of SEQ ID NO: 135.

10. The fusion polypeptide of claim 1, further comprising a nuclear localization signal (NLS).

11. The fusion polypeptide of claim 1, wherein the fusion polypeptide comprises the structure: NH2-[cytidine deaminase domain]-[first NLS]-[RNA-guided DNA binding domain]-[second NLS]-[UGI]-[third NLS]-COOH, and wherein each instance of “-” optionally comprises a linker.

12. A complex comprising the fusion polypeptide of any one of claims 1-11 and a DNA-targeting RNA bound to the RNA-guided DNA binding domain of the fusion polypeptide.

13. A cell comprising the fusion polypeptide of any one of claims 1-11.

14. The cell of claim 13, wherein the cell is a plant cell.

15. A polynucleotide encoding the fusion polypeptide of any one of claims 1-11.

16. The polynucleotide of claim 15, wherein the cytidine deaminase domain comprises a nucleotide sequence having at least 80%, at least 90%, at least 95%, at least 98%, or at least 99% sequence identity to the nucleotide sequence set forth in SEQ ID NO: 68, 72, 76, 80, 82, 83, 97, 103-106, 109-117, 119, 120, 122, 124-127, 129, or 130.

17. The polynucleotide of claim 15, wherein the polynucleotide encoding the fusion polypeptide is codon-optimized for expression in a plant cell.

18. A vector comprising the polynucleotide of claim 15.

19. The vector of claim 18, wherein the vector comprises a heterologous promotor driving expression of the polynucleotide.

20. A cell comprising the polynucleotide of claim 15.

21. A method of modifying a target nucleic acid, the method comprising:
contacting the target nucleic acid with:
(a) a fusion polypeptide comprising: (i) a cytidine deaminase domain comprising an amino acid sequence having at least 80%, at least 90%, at least 95%, at least 98%, or at least 99% sequence identity to the amino acid sequence set forth in SEQ ID NO: 2, 6, 10, 14, 16, 17, 31, 37-40, 43-51, 53, 54, 56, 58-61, 63, or 64, and (ii) an RNA-guided DNA binding domain; and
(b) a DNA-targeting RNA, wherein the DNA-targeting RNA is capable of forming a complex with the RNA-guided DNA binding domain of the fusion polypeptide and directing the complex to the target nucleic acid, resulting in one or more C to T substitutions.

22. The method of claim 21, wherein the cytidine deaminase domain comprises an amino acid sequence having at least 80%, at least 90%, at least 95%, at least 98%, or at least 99% sequence identity to the amino acid sequence set forth in SEQ ID NO: 2, 6, 14, 16, 31, 37, 38, 43, 45, 51, 53, 56, 59-61, or 63.

23. The method of claim 21, wherein the cytidine deaminase domain comprises an amino acid sequence having at least 80%, at least 90%, at least 95%, at least 98%, or at least 99% sequence identity to the amino acid sequence of SEQ ID NO: 6, 16, 37, 38, 45, or 53.

24. The method of claim 21, wherein the cytidine deaminase domain comprises the amino acid sequence of SEQ ID NO: 6, 16, 37, 38, 45, or 53.

25. The method of claim 21, wherein the RNA-guided DNA binding domain comprises a Cas9 domain, a Cas12a domain, or a Cas12b domain.

26. The method of claim 21, wherein the RNA-guided DNA binding domain is nuclease active, nuclease inactive, or a nickase.

27. The method of claim 21, wherein the RNA-guided DNA binding domain comprises an amino acid sequence having at least 80%, at least 90%, at least 95%, at least 98%, or at least 99% sequence identity to the amino acid sequence of SEQ ID NO: 133.

28. The method of claim 21, wherein the fusion polypeptide further comprises a uracil glycosylase inhibitor (UGI) domain.

29. The method of claim 28, wherein the UGI comprises an amino acid sequence having at least 80%, at least 90%, at least 95%, at least 98%, or at least 99% sequence identity to the amino acid sequence of SEQ ID NO: 135.

30. The method of claim 21, further comprising a nuclear localization signal (NLS).

31. The method of claim 21, wherein the fusion polypeptide comprises the structure: NH2-[cytidine deaminase domain]-[first NLS]-[RNA-guided DNA binding domain]-[second NLS]-[UGI]-[third NLS]-COOH, and wherein each instance of “-” optionally comprises a linker.

32. The method of claim 21, wherein the target nucleic acid is in a cell.

33. The method of claim 32, wherein the cell is a plant cell.

34. The method of claim 21, wherein the contacting is at a temperature from about 22° C. to about 32° C.

35. A method for producing a genetically modified plant, the method comprising:
introducing into the plant:
(a) the fusion polypeptide of any one of claims 1-11, or a polynucleotide encoding the fusion polypeptide; and
(b) a DNA-targeting RNA, or a DNA polynucleotide encoding the DNA-targeting RNA, wherein the DNA-targeting RNA is capable of forming a complex with the RNA-guided DNA binding domain of the fusion polypeptide and directing the complex to a target nucleic acid in the genome of the plant, resulting in one or more C to T substitutions.

36. The method of claim 35, wherein the introducing is at a temperature from about 22° C. to about 32° C.

37. The method of claim 35, wherein the plant is a monocotyledonous or a dicotyledonous species.

38. The method of claim 35, wherein the plant is Oryza sativa or Solanum lycopersicum.
